(19) United States
(12) Patent Application Publication
(10) Pub. No.: US 2022/0259580 A1
(43) Pub. Date: Aug. 18, 2022

(54) ENZYMATIC HEXOSAMINIDATION OF LACTOSE

(71) Applicant: BASF SE, Ludwigshafen (DE)

(72) Inventors: Kai-Uwe Baldenius, Mannheim (DE); Michael Breuer, Ludwigshafen (DE); Corinna Ruffer, Ludwigshafen (DE); Melanie Weingarten, Ludwigshafen (DE); Reinhard Zschoche, Ludwigshafen (DE); Stefan Seemayer, Ludwigshafen (DE); Bernd Nidetzky, Graz (AT); Katharina Schmoelzer, Graz (AT); Michael Puhl, Ludwigshafen (DE)

(21) Appl. No.: 17/416,480

(22) PCT Filed: Dec. 9, 2019
(86) PCT No.: PCT/EP2019/084216

§ 371 Date: Jun. 19, 2021

(30) Foreign Application Priority Data
Dec. 20, 2018 (EP) 18214455.0

(51) Int. Cl. C12N 9/24; C12P 19/14; C12P 19/18; C12N 1/20

(52) U.S. Cl. CPC C12N 9/2402; C12P 19/14; C12N 1/205; C12Y 302/0114; C12P 19/18

(57) ABSTRACT

The present disclosure relates to methods for producing human milk oligosaccharide (HMO) core structures using glycosidases from family GH20 hexosaminidases. In particular, the present disclosure provides methods for producing lacto-N-triose II (LNT II) and/or lacto-N-tetraose (LNT) by reacting glucosamine-oxazoline and/or lacto-N-biose-oxazoline with lactose catalysed by an enzyme of the glycoside hydrolase family 20 (GH20) according to the classification of the Carbohydrate-Active-Enzymes (CAZy) database. Specific optimized enzymes are identified to catalyse the reactions.

### CROSS-REFERENCE TO RELATED APPLICATIONS

The present application is a 371 U.S. National Phase Patent Application based on International Application No. PCT/EP2019/084216, filed Dec. 9, 2019, which claims the benefit of European Patent Application No. EP18214455.0, filed Dec. 20, 2018, the entire disclosures of which are hereby expressly incorporated by reference herein.

### REFERENCE TO A SEQUENCE LISTING

The present application contains a sequence listing which has been submitted in ASCII format via EFS-Web and is hereby incorporated by reference in its entirety. Said ASCII copy, created on Feb. 3, 2022 is named 074008-000262-SubSL2.txt and is 268,304 bytes in size.

### BACKGROUND/SUMMARY

The present disclosure relates to methods for producing human milk oligosaccha-ride (HMO) core structures using glycosidases from family GH20 hexosaminidases. In particular, the present disclosure provides methods for producing lacto-N-triose II (LNT II) and/or lacto-N-tetraose (LNT) by reacting glucosamine-oxazoline and/or lacto-N-biose-oxazoline with lactose catalysed by an enzyme of the glycoside hydrolase family 20 (GH20) according to the classification of the Carbohydrate-Active-Enzymes (CAZy) database. Specific optimized enzymes are identified to catalyse the reactions. Furthermore, the present disclosure also relates to the use of enzymes of the glycoside hydrolase family 20 (GH20) for producing lacto-N-triose II or lacto-N-tetraose.

Different endo-type glycosidases (e.g. Endo M, Endo A, chitinases) were applied for synthesis of the N-linked oligosaccharide core trisaccharide Man(β-4)-GIcNAc(β1-4)-GlcNAc and derivatives thereof, using the corresponding disaccharide oxazoline as a donor substrate.^{1, 2} The N-linked oligosaccharide core trisaccharide Man(β1-4)-GIcNAc(β1-4)-GlcNAc was obtained only in 32% yield (6.4 mM) from disaccharide-oxazoline in the chitinase-catalyzed reaction, although a large excess of the acceptor substrate (32-fold) and 20% of acetone (v/v) were used.²Artificial chitin and other glycosaminoglycan derivatives have been synthesized by polymerization of modified disaccharide oxazolines using family GH18 chitinase and GH56 hyaluronidase as the catalyst.³ The polymerization of disaccharide oxazoline with Endo A was also described.⁴

Hexosaminidases are hydrolases with often very low trans-glycosylation activity. In order to solve the hydrolysis problem and provide attractive yields, enzymes from different glycosid hydrolase families have been mutated to enhance trans-glycosylation activity and decrease or suppress hydrolysis. When optimizing the specific activity of glycoside hydrolases, however, also suitable donor-substrates and reaction conditions for the trans-glycosylation need to be identified to achieve high yields.

The hexosaminidases Bbhl (β-N-acetylhexosaminidase) and LnbB (lacto-N-bio-sidase) from B. bifidum JCM1254 are members of the glycoside hydrolase family 20 (GH20) according to the classification of the Carbohydrate-Active-Enzymes (CAZy) database. The Carbo-hydrate -Active-Enzymes database is available at http://www.cazy.org in the version updated on 20 Nov. 2018 (Lombard, V.; Golaconda Ramulu, H.; Drula, E.; Coutinho, P. M.; Henrissat, B., The carbohydrate-active enzymes database (CAZy) in 2013, Nucleic Acids Res., 2014, 42 (D1), D490-D495.). The database for automated carbohydrate-active enzyme annotation (dbCAN) provides annotation data, which is generated based on the family classification from CAZy database (http://csbl.bmb.uga.edu/dbCAN/index.php, Yin Y, Mao X, Yang JC, Chen X, Mao F and Xu Y, dbCAN: a web resource for automated carbohydrate-active enzyme annotation, Nucleic Acids Res. 2012 Jul.; 40(Web Server issue):W445-51).The glycosylations to produce LNT II and LNT are shown in Scheme 1. Recently, Xiao and co-workers demonstrated biocatalytic synthesis of LNT II at moderate yield (37%) by trans-glycosylation, using wild-type Bbhl.⁵ However, despite intensive reaction optimization (substrate concentration, organic solvent concentration, pH, temperature), the maximum yield was strongly compromised due to low donor to acceptor ratios and due to secondary hydrolysis of the LNT II. LnbB also showed trans-glycosylation activity, but LNT was obtained only in very low yield (<4%).⁶ Other researchers have tried to improve the transglycosylation of lactose with N-Ac-glucosamine from N,N-di-acetylchitobiose by introduction of certain mutations into GH20 hexosaminidases, but only managed to increase the LNT II yield from 0.5% to 5% while LNT II remained contaminated with at least two isomeric trisaccharides.⁷

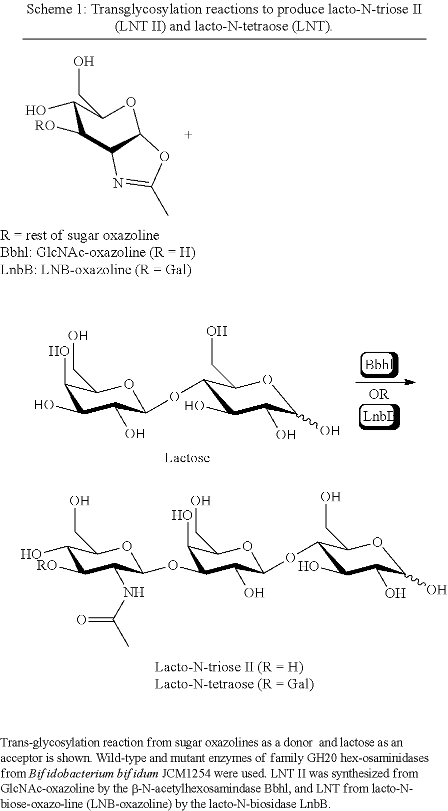

It was an objective of the present disclosure to provide techniques to improve the commercial usability of the trans-glycosylation of lactose to LNT II and LNT by Bbhl and LnbB through optimizing the specific activity of the enzymes as well as identifying suitable donor-substrates and reaction conditions. In particular, it was an objective of the present disclosure to increase the yield, the final product concentration and the space-time-yield of the transglycosylation compared to the methods described in the prior art.

The objective is met by a method for producing lacto-N-triose II and/or lacto-N-tetraose comprising the step:
(i) reacting glucosamine-oxazoline and/or lacto-N-biose-oxazoline with lactose catalysed by an enzyme of the glycoside hydrolase family 20 (GH20) according to the classification of the Carbohydrate-Active-Enzymes (CAZy) database (http://www.cazy.org, updated on 20 Nov. 2018) to obtain lacto-N-triose II and/or lacto-N-tetraose.

Preferably, step (i) is performed under conditions suitable for the production of lacto-N-triose II or lacto-N-tetraose or both.

In a preferred embodiment of the method according to the present disclosure, glucosamine -oxazoline and/or lacto-N-biose-oxazoline is contacted with lactose and at least one enzyme of the glycoside hydrolase family 20 (GH20) according to the classification of the Carbohydrate -Active-Enzymes (CAZy) database (http://www.cazy.org, updated on 20 Nov. 2018) under conditions suitable for the formation of lacto-N-triose II or lacto-N-tetraose or both. It was found in the context of the present disclosure, that the reaction of an enzyme of the glycoside hydrolase family 20 (GH20) according to the classification of the Carbohydrate-Active-Enzymes (CAZy) database with lactose and an oxazoline donor-substrate provides a promising way to obtain HMO core structures in suitable yields.

Glycoside hydrolase family 20 (GH20) enzymes are enzymes of the EC 3.2.1.- class according to the EC classification of enzymes (www.brenda-enyzmes.org, Schomburg et al., The BRENDA enzyme information system - From a database to an expert system, J Biotech-nol, 2017, 261, 194-206; www.expasy.org, Artimo et al., ExPASy: SIB bioinformatics resource portal, Nucleic Acids Res. 40(W1): W597-W603, 2012). In the context of the present disclosure, an enzyme of the glycoside hydrolase family 20 (GH20) is an enzyme that is capable to perform a trans-glycosylation reaction. Preferably an enzyme of the glycoside hydrolase family 20 (GH20) is an enzyme of the EC class 3.2.1.52 or EC class 3.2.1.140, and more preferably a β-N -acetylhexosaminidase or a lacto-N-biosidase, even more preferably a β-N-acetylhexosamini-dase.

The catalytic activity in this family of enzymes is assigned to the GH20 domain (PFAM code PF00728) which typically folds into a (β/α)₈-barrel topology. Except for dispersin B from A. actinomycetemcomitans that presents only a single GH20 domain, the rest of structures show the catalytic domain accompanied by several domains with quite diverse functionalities: a non-catalytic domain, commonly named as GH20b, which is conserved in most GH20 enzymes although with unknown function, several lectin domains, carbohydrate binding domains, and other domains of unknown function.³⁴

In a preferred embodiment of the various aspects of the present disclosure, an enzyme of the glycoside hydrolase family 20 (GH20) is an enzyme, which comprises a domain having the PFAM code PF00728 according to the Pfam database (https://pfam.xfam.org, Pfam 32.0, September 2018).

Sugar oxazolines are attractive donor substrates for trans-glycosylation by retaining glycosidases, which follow a substrate-assisted reaction mechanism. They represent a highly active substrate species mimicking the transition state. Shoda and co-workers demonstrated their practical one-step synthesis in good yield (>80%) from unprotected N-acetyl-2-amino sugars in water by using 2-chloro-1,3-dimethyl-1H-benzimidazol-3-ium chloride (CDMBI) as a dehydrative agent and Na₃PO₄ as a base.⁸ Practical preparation of lacto-N-biose (LNB) in bulk quantities from sucrose and N-Acetylglucosamine (GlcNAc), using a one-pot four-enzyme reaction with lacto-N-biose phosphorylase (LNBP) from Bifidobacterium bifidum as a key enzyme, was reported by Kitaoka and co-workers.^{9,10} However, so far, oxazoline donor substrates have not been successfully employed for transglycosylation with a GH20 enzyme. In Slámová et al., it is explained that the oxazoline ring is very unstable depending on the pH and it is described that no transglycosylation products were observed in the reaction of β-N-acetylhexosaminidases with oxazoline donor substrates.¹¹

In a preferred embodiment of the present disclosure, in the method described above, step (i) is performed in an aqueous solution containing at least 50 g/L lactose, preferably at least 100 g/L lactose, particularly preferably at least 150 g/L lactose, most preferably at least 190 g/L lactose.

Surprisingly, it was found in the context of the present disclosure, that a high lactose concentration does not negatively influence the product yield despite the increasing viscosity. At the same time, the high concentrations allow an easier scale-up because of the smaller volumes that need to be handled.

Particularly preferred is a method as described above, which is performed under conditions that are free or essentially free of organic solvents.

“Essentially free” in this context preferably means that at any given point in time, in the reaction mixture of step (i) and, in particular in the purified and not purified product obtained by the method described above, there is 10 g/L or less, more preferably 1 g/L or less of an organic solvent present. The use of organic solvents such as DMSO or Ethanol is undesirable in any product intended for human consumption, especially in baby food, which represents a main application for the present disclosure. Advantageously, the method according to the present disclosure allows production of HMO core structures under reaction conditions that are free or essentially free of organic solvents.

In a further preferred embodiment of the present disclosure, in the method described above, the glucosamine-oxazoline and/or lacto-N-biose-oxazoline, respectively, is added to the lactose and the enzyme of the glycoside hydrolase family 20 (GH20) over a period of at least 20 minutes, preferably at least 60 minutes.

The glucosamine-oxazoline and/or lacto-N-biose-oxazoline may be added in batches over the respective period of time or, preferably, are added continuously over the respective period of time.

In one preferred embodiment of the method for producing lacto-N-triose II according to the disclosure, the enzyme of the glycoside hydrolase family 20 (GH20) is a β-N-acetylhex-osaminidase, preferably β-N-acetylhexosaminidase of Bifidobacterium bifidum JCM1254 having an amino acid sequence of SEQ ID NO: 1 (Bbhl, GenBank accession number: AB504521.1) or an enzyme having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 1 and having β-N-acetylhexosaminidase activity, or a β-N-acetylhexosaminidase enzyme comprising an amino acid sequence of SEQ ID NO: 2, or a β-N-acetylhexosaminidase enzyme comprising an amino acid sequence having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 2.

In one preferred embodiment of the method for producing lacto-N-tetraose according to the disclosure, the enzyme of the glycoside hydrolase family 20 (GH20) is a lacto-N-bio-sidase, preferably lacto-N-biosidase of Bifidobacterium bifidum JCM1254 having an amino acid sequence of SEQ ID NO: 3 (LnbB, GenBank accession number: EU281545.1), or an enzyme having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 3 and having lacto-N-biosidase activity, or a lacto-N-biosidase enzyme comprising an amino acid sequence of SEQ ID NO: 4, or a lacto-N-biosidase enzyme comprising an amino acid sequence having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 4.

“Identity” in relation to comparison of two amino acid sequences herein is calculated by dividing the number of identical residues by the length of the alignment region, which is showing the shorter sequence over its complete length. This value multiplied by 100 gives “%-identity”. To determine the %-identity between two amino acid sequences (i.e. pairwise sequence alignment), two sequences have to be aligned over their complete length (i.e. global alignment) in a first step. For producing a global alignment of two sequences, any suitable computer program, like program “NEEDLE” (The European Molecular Biology Open Software Suite (EMBOSS)), program “MATGAT” (Campanella, J. J, Bitincka, L. and Smalley, J. (2003), BMC Bioinformatics, 4:29), program “CLUSTAL” (Higgins, D:G: and Sharp, P. M. (1988), Gene, 73, 237-244), program “MegAlign Pro” (DNASTAR) or similar programs may be used. In lack of any program, sequences may also be aligned manually. After aligning two sequences, in a second step, an identity value shall be determined from the alignment. Depending on the applied method for %-identity calculation, different %-identity values can be calculated from a given alignment. Consequently, computer programs which create a sequences alignment, and in addition calculate %-identity values from the alignment, may also report different %-identity values from a given alignment, depending which calculation method is used by the program. Therefore, the following calculation of %-identity according to the disclosure applies: %-identity=(identical residues/length of the alignment region which is showing the shorter sequence over its complete length) *100. The calculation of %-identity according to the disclosure is exemplified as follows (the sole purpose of SEQ ID NO: 26 and SEQ ID NO 27 is to demonstrate calculation according to the disclosure; besides this purpose, said sequences are not inventive or functionally meaningful):

SEQ ID NO 26:
TTTTTTAAAAAAAACCCCHHHCCCCAAARVHHHHHTTTTTTTT-
length: 43 amino acids
SEQ ID NO 27:
TTAAAAAAAACCCCHHCCCCAAADLSSHHHHHTTTT-length:
36 amino acids

Hence, the shorter sequence is sequence 2.

Producing a pairwise global alignment which is showing both sequences over their complete lengths results in

TTTTTTAAAAAAAACCCCHHHCCCCAAARV--HHHHHTTTTTTTT
|||||||||||||| ||||||||| ; |||||||||
----TTAAAAAAAACCCC-HHCCCCAAADLSSHHHHHTTTT----

Producing a pairwise alignment which is showing the shorter sequence over its complete length according the disclosure consequently results in:

TTAAAAAAAACCCCHHHCCCCAAARV--HHHHHTTTT
|||||||||||||| ||||||||| ; |||||||||
TTAAAAAAAACCCC-HHCCCCAAADLSSHHHHHTTTT

The number of identical residues is 32, the alignment length showing the shorter sequence over its complete length is 37 (one gap is present which is factored in the alignment length of the shorter sequence). Therefore, %-identity according to the disclosure is: (32/37)*100=86%.

The enzymes used in the context of the present disclosure may have 1, 2, 3, 4, 5, 6, 7, 8, 9 or 10 conservative amino acid substitutions.

Preferably, the enzymes used in the context of the present disclosure have non-naturally occurring amino acid sequences and represent artificial constructs, which have been taken out of their natural biological context and have been specifically designed for the purpose as described herein. They may therefore be truncated, but functional versions of naturally occurring proteins and may carry certain modifications, for example those that simplify their bio-technological production or purification, e.g. a His-tag.

The disclosure also provides nucleotide sequences, in particular DNA sequences, and methods as described above making use of such nucleotide sequences, wherein the nucleotide sequences hybridize under stringent conditions with a DNA or RNA sequence, which codes for an enzyme of the glycoside hydrolase family GH20 as described above, in particular a β-N-acetylhexosaminidase or a lacto-N-biosidase according to SEQ ID NO: 1 or 3 or a truncated functional version of the respective enzymes according to SEQ ID NO: 2 or 4.

The term “hybridisation” as defined herein is a process wherein substantially complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can further-more occur with one of the complementary nucleic acids immobilised to a solid support such as a nitro-cellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

This formation or melting of hybrids is dependent on various parameters, for example the temperature. An increase in temperature favours melting, while a decrease in temperature favours hybridisation. However, this hybrid forming process is not following an applied change in temperature in a linear fashion: the hybridisation process is dynamic, and already formed nucleotide pairs are supporting the pairing of adjacent nucleotides as well. So, with good approximation, hybridisation is a yes-or-no process, and there is a temperature, which basically defines the border between hybridisation and no hybridisation. This temperature is the melting temperature (Tm). Tm is the temperature in degrees Celsius, at which 50% of all molecules of a given nucleotide sequence are hybridised into a double strand, and 50% are present as single strands.

The melting temperature (Tm) is dependent from the physical properties of the analysed nucleic acid sequence and hence can indicate the relationship between two distinct sequences. However, the melting temperature (Tm) is also influenced by various other parameters, which are not directly related with the sequences, and the applied conditions of the hybridization experiment must be taken into account. For example, an increase of salts (e.g. monovalent cations) is resulting in a higher Tm.

Tm for a given hybridisation condition can be determined by doing a physical hybridisation experiment, but Tm can also be estimated in silico for a given pair of DNA sequences. In this embodiment, the equation of Meinkoth and Wahl (Anal. Biochem., 138:267-284, 1984) is used for stretches having a length of 50 or more bases:

Tm=81.5° C.+16.6 (log M)+0.41 (% GC)−0.61 (% form)−500/L

M is the molarity of monovalent cations, % GC is the percentage of guanosine and cytosine nucleotides in the DNA stretch, % form is the percentage of formamide in the hybridisation solution, and L is the length of the hybrid in base pairs. The equation is for salt ranges of 0.01 to 0.4 M and % GC in ranges of 30% to 75%.

While above Tm is the temperature for a perfectly matched probe, Tm is reduced by about 1° C. for each 1% of mismatching (Bonner et al., J. Mol. Biol. 81: 123-135, 1973):

Tm=[81.5° C.+16.6(log M)+0.41 (% GC)−0.61 (% formamide)−500/] %non-identity

This equation is useful for probes having 35, preferably 50 or more nucleotides and is widely referenced in scientific method literature (e.g. in: “Recombinant DNA Principles and Methodologies”, James Greene, Chapter “Biochemistry of Nucleic acids”, Paul S. Miller, page 55; 1998, CRC Press), in many patent applications (e.g. in: U.S. Pat. No. 7,026,149), and also in data sheets of commercial companies (e.g. “Equations for Calculating Tm” from www.genomics.agilent.com).

For an in silico estimation of Tm according to this embodiment, first a set of bioinformatic sequence alignments between the two sequences are generated. Such alignments can be generated by various tools known to a person skilled in the art, like programs “Blast” (NCBI), “Water” (EMBOSS) or “Matcher” (EMBOSS), which are producing local alignments, or “Needle” (EMBOSS), which is producing global alignments. Those tools should be applied with their default parameter setting, but also with some parameter variations. For example, program “MATCHER” can be applied with various parameter for gapopen/gapextend (like 14/4; 14/2; 14/5; 14/8; 14/10; 20/2; 20/5; 20/8; 20/10; 30/2; 30/5; 30/8; 30/10; 40/2; 40/5; 40/8; 40/10; 10/2; 10/5; 10/8; 10/10; 8/2; 8/5; 8/8; 8/10; 6/2; 6/5; 6/8; 6/10) and program “WATER” can be applied with various parameter for gapopen/gapextend (like 10/0,5; 10/1; 10/2; 10/3; 10/4; 10/6; 15/1; 15/2; 15/3; 15/4; 15/6; 20/1; 20/2; 20/3; 20/4; 20/6; 30/1; 30/2; 30/3; 30/4; 30/6; 45/1; 45/2; 45/3; 45/4; 45/6; 60/1; 60/2; 60/3; 60/4; 60/6), and also these programs shall be applied by using both nucleotide sequences as given, but also with one of the sequences in its reverse complement form. For example, BlastN (NCBI) can be applied with an increased e-value cut-off (e.g. e+1 or even e+10) to also identify very short alignments, especially in data bases of small sizes.

Important is that local alignments are considered, since hybridisation may not necessarily occur over the complete length of the two sequences, but may be best at distinct re-gions, which then are determining the actual melting temperature. Therefore, from all created alignments, the alignment length, the alignment % GC content (in a more accurate manner, the % GC content of the bases which are matching within the alignment), and the alignment identity has to be determined. Then the predicted melting temperature (Tm) for each alignment has to be calculated. The highest calculated Tm is used to predict the actual melting temperature.

The term “hybridisation over the complete sequence of the disclosure” as defined herein means that when the sequence of the disclosure is fragmented into pieces of about 300 to 500 bases length, every fragment must hybridise. For example, a DNA can be fragmented into pieces by using one or a combination of restriction enzymes. A bioinformatic in silico calculation of Tm is then performed by the same procedure as described above, just done for every fragment. The physical hybridisation of individual fragments can be analysed by standard Southern analysis, or comparable methods, which are known to a person skilled in the art.

The term “stringency” as defined herein is describing the ease by which hybrid formation between two nucleotide sequences can take place. Conditions of a “higher stringency” require more bases of one sequence to be paired with the other sequence (the melting temperature Tm is lowered in conditions of “higher stringency”), conditions of “lower stringency” allow some more bases to be unpaired. Hence the degree of homology between two sequences can be estimated by the actual stringency conditions at which they are still able to form hybrids. An increase in stringency can be achieved by keeping the experimental hybridisation temperature constant and lowering the salts concentrations, or by keeping the salts constant and increasing the experimental hybridisation temperature, or a combination of these parameter. Also, an increase of formamide will increase the stringency. The skilled artisan is aware of additional parameters which may be altered during hybridisation and which will either maintain or change the stringency conditions (Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

A typical hybridisation experiment is done by an initial hybridisation step, which is followed by one to several washing steps. The solutions used for these steps may contain additional components, which are preventing the degradation of the analyzed sequences and/or prevent unspecific background binding of the probe, like like EDTA, SDS, fragmented sperm DNA or similar reagents, which are known to a person skilled in the art (Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

A typical probe for a hybridisation experiment is for example generated by the random -primed-labeling method, which was initially developed by Feinberg and Vogelstein (Anal. Biochem., 132 (1), 6-13 (1983); Anal. Biochem., 137 (1), 266-7 (1984) and is based on the hybridisation of a mixture of all possible hexanucleotides to the DNA to be labeled. The labeled probe product will actually be a collection of fragments of variable length, typically ranging in sizes of 100-1000 nucleotides in length, with the highest fragment concentration typically around 200 to 400 bp. The actual size range of the probe fragments, which are finally used as probes for the hybridisation experiment, can for example also be influenced by the used labeling method parameter, subsequent purification of the generated probe (e.g. agarose gel), and the size of the used template DNA which is used for labeling (large templates can e.g. be restriction digested using a 4 bp cutter, e.g. Haelll, prior labeling).

A person of skill is aware that the hybridization under stringent conditions will depend on the % GC of the sequence, and that depending on codon usage and GC content optimization, nucleotides encoding the same amino acid sequence may have varying % GC to each other. For a particular nucleotide sequence of interest, it hence can be determined by knowledge and skill what hybridization and washing conditions, buffers and salts have to be used for stringent conditions, based on the knowledge about the sequences to be compared by hybridization and the knowledge on hybridization in the art. For example, for a nucleotide sequence of % GC of 55 and of an average length of the oligo probes of 300 bp after random-primed labelling, an SSC buffer with no more than 1.82×SSC and 0.1% SDS and at least 65° C. can be used for stringent conditions. If even more stringent conditions are to be used, the concertation of SSC is to be lowered and in addition up to 5 to 10% (v/v) of formamide can be added.

The enzymes of the glycoside hydrolase family GH20 may be used in the method according to the present disclosure in free or immobilized form. They may e.g. be attached to a suitable carrier or beads or similar structures.

The hexosaminidases Bbhl (β-N-acetylhexosaminidase) and LnbB (lacto-N-bio-sidase) from B. bifidum JCM1254 were identified as promising candidates for the enzymatic synthesis of human milk oligosaccharide (HMO) core structures. β-N-acetylhexosaminidase Bbhl belongs to the enzyme class EC 3.2.1.52 and lacto-N-biosidase LnbB to the enzyme class EC 3.2.1.140. The sequences of the wild type enzymes from Bifidobacterium bifidum JCM1254 can be found in the GenBank database under the accession number AB504521.1 (published on Mar. 31, 2010), which corresponds to SEQ ID NO: 1, and EU281545.1 (published on Jul. 1, 2008), which corresponds to SEQ ID NO: 3. Exemplary truncated constructs of the enzymes, which are fully functional, are represented by SEQ ID NO: 2 (truncated Bbhl, aa 33-1599) and SEQ ID NO: 4 (truncated LnbB, aa 35-1064).

Bbhl was previously described by Chen, X., et al. in “Efficient and regioselective synthesis of β-GaINAc/GIcNAc-lactose by a bifunctional transglycosylating β-N-acetylhex-osaminidase from Bifidobacterium bifidum” Appl. Environ. Microbiol. 82, 5642-5652 (2016).⁵ The Enzyme was obtained from genomic DNA of B. bifidum JCM 1254. Furthermore, Miwa, M., et al. (Cooperation of β-galactosidase and β-N-acetylhexosaminidase from bifidobacteria in assimilation of human milk oligosaccharides with type 2 structure. Glycobiology 20, 1402-1409 (2010)) used Bbh1 amplified by PCR using genomic DNA from B. bifidum JCM1254 as template.¹² The bifidobacterial strain was obtained from the Japan Collection of Microorganisms (JCM), RIKEN Bioresource Center, Japan.

LnbB was previously described in Wada, J., et al. “Bifidobacterium bifidum lacto-N-biosidase, a critical enzyme for the degradation of human milk oligosaccharides with a type 1 structure”, Appl. Environ. Microbiol. 74, 3996-4004 (2008).⁶ LnbB was amplified by PCR using genomic DNA from B. bifidum JCM1254 as a template. The bacterial strains were obtained from the Japan Collection of Microorganisms (JCM), RIKEN Bioresource Center, Japan.

In order to increase the trans-glycosylation activity with respect to the hydrolase activity, different mutations were tested. It was found that replacement in SEQ ID NO: 1 of Asp⁷⁴⁶ by Ala, Glu or Gln gave very noticeable improvement with respect to secondary hydrolysis and little or no loss in productive activity. Purified preparations of wild-type and variant enzymes were obtained from Escherichia coli overexpression cultures, with a C-terminal His6-tag for purification by metal chelate chromatography.

In a preferred embodiment, in the method for producing lacto-N-triose II described above, the β-N-acetylhexosaminidase has an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 1 (Bbhl, GenBank accession number: AB504521.1) and carries a mutation at a position counted as position 746 of SEQ ID NO: 1 and/or at a position counted as position 827 of SEQ ID NO: 1, preferably carries a glutamic acid or an alanine or a glutamine at a position counted as position 746 of SEQ ID NO: 1 and/or carries a phenylalanine at a position counted as position 827 of SEQ ID NO: 1, or the β-N-acetylhexosaminidase comprises an amino acid sequence of any of SEQ ID NOs: 5 to 8 or comprises an amino acid sequence having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to any of SEQ ID NOs: 5 to 8.

SEQ ID NOs: 5 to 8 represent the truncated versions of Bbhl carrying the above described mutations. The mutations at position 746 and 827 of SEQ ID NO: 1 are not to be understood as absolute, but corresponding mutations at equivalent positions of enzymes belonging to the GH20 family or to the EC class EC 3.2.1.52 and having a glycosidase activity, in particular β-N-acetylhexosaminidases, e.g. homolog enzymes from other organisms, also provide the functionality according to the disclosure. Equivalent positions can be identified by a global sequence alignment as shown in FIG. 16.

Preferably, the Bbhl used in the context of the present disclosure has a non-naturally occurring amino acid sequence and represents an artificial construct, which has been taken out of its natural biological context and has been specifically designed for the purpose as described herein. It may therefore be a truncated, but functional version of the naturally occurring protein and may carry certain modifications, for example those that simplify its biotechnological production or purification, e.g. a His-tag.

The disclosure also provides nucleotide sequences, in particular DNA sequences, and methods as described above making use of such nucleotide sequences, wherein the nucleotide sequences hybridize under stringent conditions with a DNA or RNA sequence, which codes for an enzyme of the glycoside hydrolase family GH20 as described above, in particular a β-N-acetylhexosaminidase carrying a mutation at a position counted as position 746 of SEQ ID NO: 1 and/or at a position counted as position 827 of SEQ ID NO: 1, preferably carrying a glutamic acid or an alanine or a glutamine at a position counted as position 746 of SEQ ID NO: 1 and/or carrying a phenylalanine at a position counted as position 827 of SEQ ID NO: 1 or a truncated functional version of the enyzme according to any of SEQ ID NO: 5 to 8.

The trans-glycosylation activities of the Bbhl enzymes in the LNT II synthesis reaction from GlcNAc-oxazoline or GlcNAc-β-pNP onto lactose were assayed. In a first step, a 10- or 20-fold excess of the acceptor substrate was used to promote product formation. FIG. 1 (Glc-NAc -oxazoline) and the FIG. 2 (GlcNAc-β-pNP) compare reaction time courses for the Bbhl variants to that of the wild-type enzyme. All product concentrations were observed analytically by HPLC analysis with UV detection at 195 nm (FIG. 3). The elution of the N-acetyl-2-amino sugars (LNT II, GlcNAc) could be monitored by measuring the absorbance of the N-acetyl-group. Table 1 summarizes the specific activities (trans-glycosylation, secondary hydrolysis) and selectivity parameters (RTH value) of the Bbhl enzymes. In order to quantify the change in reaction selectivity under “synthesis conditions” in the presence of acceptor substrate, the parameter RTH (ratio of the specific trans-glycosylation activity of GlcNAc-to-lactose transfer to the specific activity of non-productive donor substrate hydrolysis (primary hydrolysis) in the presence of acceptor substrate) was used. Using wild-type Bbhl and GlcNAc-oxazoline as a donor (FIG. 1a), LNT II was obtained in a maximum yield of about 52% (34 mM). The D746E mutant showed a significantly enhanced trans-glycosylation activity. The maximum LNT II yield was 87% (53 mM) (FIG. 1b). The Y827F mutant (FIG. 1c) showed a similar behavior as the D746E variant, giving a maximum yield of 80%. The Asp to Ala mutation at residue 746 completely abolished the hydrolysis activity (FIG. 1d) of Bbhl, but the trans-glycosylation activity was also reduced compared to the wild-type. Replacement of the Asp⁷⁴⁶ by Gln resulted in almost complete abolishment of both secondary hydrolysis and trans-glycosylation activities (FIG. 1e). The enzymes' inherent hydrolase activity caused also partly non-productive utilization of the GlcNAc-oxazoline substrate, thus restricting the LNT II yield under the conditions used. The D746E mutant exhibited a significantly enhanced trans-glycosylation activity over the primary hydrolysis, with a RTH value of 50 (FIG. 1f, Tab. 1). Comparable results to the LNT II synthesis from GlcNAc-oxazoline were obtained with GlcNAc-p-pNP as a donor substrate (Tab. 1, FIG. 2). In all cases, formation of LNT II was confirmed by TLC analyses (FIGS. 2f, 4). In general, higher trans-glycosylation activities were observed with GlcNAc-oxazoline. Note, non-enzymatic hydrolysis of GlcNAc-oxa-zoline (ring opening of the oxazoline moiety) can also occur, depending on the pH.^{4,13} It was known from literature that the pH value strongly affected product formation by wild-type Bbhl using GlcNAc-p-pNP.⁵ The optimum pH in terms of product yield was at pH 5.8, with a sharp decrease at pH 9. Although sugar oxazolines have a higher stability at relatively high pH (>8, data not shown), pH 7.5 represented a good compromise between enzyme activity and donor substrate stability. Maximum yields were obtained within 10 min-120 min. Note, using a 3.2-fold excess of GlcNAc-oxazoline over lactose, to counteract enzymatic and non-enzymatic Glc-NAc -oxazoline hydrolysis, caused poor LNT II yields: The maximum yield was only 32% with the wild-type and 54% with the D746E variant (FIG. 5).

TABLE 1
Activity and selectivity parameters of wild-type
and site-directed variants of Bbhl

| | GlcNAc-oxazoline^{a} | | | GlcNAc-β-pNP^{b} | | |
|---|---|---|---|---|---|---|
| | Trans- | | | Trans- | | |
| | gly- | | Secondary | gly- | | Secondary |
| | cosylation | | hydrolysis | cosylation | | hydrolysis |
| Bbhl | (U mg⁻¹) | R_{T/H}^{c} | (U mg⁻¹) | (U mg⁻¹) | R_{T/H}^{c} | (U mg⁻¹) |
| WT | 38 | 3 | 1.9 | 12 | 3 | 1.1 |
| D746E | ≥3.7^{d} | 50 | 6.0 × 10⁻² | 4.4 × 10⁻¹ | 40 | 2.7 × 10⁻² |
| D746A | 2.2 × 10⁻¹ | 8 | n.d. | 1.0 × 10⁻² | 6 | n.d. |
| D746Q | 2.6 × 10⁻² | ≥1 | n.d. | 1.3 × 10⁻³ | 2 | n.d. |
| Y827F | 2.4 | 8 | 6.3 × 10⁻² | 2.6 × 10⁻¹ | 5 | 1.9 × 10⁻² |

n.d., not detectable.
^{a}60 mM GlcNAc-oxazoline, 600 mM lactose, 37° C., pH 7.5.
^{b}20 mM GlcNAc-β-pNP, 400 mM lactose, 20% DMSO, 55° C., pH 5.8.
^{c}R_{TH} is the ratio of the specific trans-glycosylation activity of GlcNAc-to-lactose transfer to the specific activity of non-productive donor substrate hydrolysis (GlcNAc-oxazoline or GlcNAc-β-pNP; primary hydrolysis) under ‘synthesis conditions’.
^{d}First data point was most probably outside the linear range.

Overall, Tab. 1 and FIG. 1f clearly show that the D746E variant of the β-N-acetylhex-osaminidase Bbhl was the best candidate for further optimization of LNT II synthesis. With Glc-NAc -oxazoline as a donor substrate, this mutant had a specific trans-glycosylation activity of ≥3.7 U mg⁻¹ and showed strong preference for trans-glycosylation, as compared to primary and secondary hydrolysis. Compared to the reaction with GlcNAc-β-pNP, the trans-glycosylation activity was ˜10-fold increased and no toxic p-nitrophenol was released as a by-product. LNT II synthesis from GlcNAc-β-pNP by wild-type Bbhl could be reproduced.⁵ Mutants of chitinases from family GH18 were previously shown to have enhanced trans-glycosylation activity for chitooligomer synthesis, whereas the hydrolysis activity for the product was diminished.^{14,15} So far, only two engineered GH20 hexosaminidases with improved trans-glycosylation activities are known.^{11,7} Among them, a fungal chitinase with the Tyr470 replaced by Phe did not accept GlcNAc-oxazoline as a donor substrate in trans-glycosylation reaction.¹¹ LNT II synthesis from N,N′-diacetylchitobiose as a donor substrate by loop mutants of the β-N-acetylhexosaminidase HEX1 from metagenomic origin suffered from low yields (≤30%).⁷ Furthermore, four regioisomers were formed and LNT II (4.7% yield) was not the main product. The present disclosure provides for the first time a GH20 glycosidase-like enzyme, which is as a useful catalyst for small molecule synthesis from sugar oxazoline.

The D746E mutant of Bbhl shows remarkable trans-glycosylation activity with only marginal or no product hydrolysis activity. The combined use of this glycosidase mutated enyzme with a highly activated donor substrate, the sugar oxazoline, resulted in a 60-fold enhanced LNT II concentration when compared to the reference reaction with wild-type Bbhl and GlcNAc-β-pNP.⁵ The yield was doubled (based on donor and acceptor substrate) and the TTNmass value (388 g_{LNT II gBbhl-1)} was 1.6-fold improved at a 170-fold increased STY (562 g L⁻¹ h⁻¹). Further intensification of LNT II synthesis from GlcNAc-oxazoline could be realized by changing from batch to a continuous process mode. Benchmarked against the best LNT II syntheses using N-acetylglucosaminyltransferases, either in vitro or in vivo, the LNT II concentration of 515 mM represents an improvement by one to two orders of magnitude.^{16, 17, 18, 19, 20, 21, 22, 23} The TTNmass was significantly enhanced (2- to 26-fold) compared to the in vitro syntheses of LNT II, using one-pot multienzyme systems for monosaccharide activation and the transfer pro-cess.^{19,21} So far, large scale production of LNT II could only be achieved with glycosyltransfer-ase -based in vivo systems (whole-cell or fermentation-based).^{17, 20, 24} However, much larger reaction volumes (20- to 100-fold) are required to obtain the product in quantities comparable to the transglycosylation process presented herein.^{17, 20, 21}

For the first time, a readily scalable transglycosylation process is provided by the present disclosure, allowing the production of a small molecule, the HMO core structure LNT II, from a sugar oxazoline in bulk quantities. So far, there is only one report of a GH18 chitinase, showing the potential of a glycosidase for the synthesis of small molecules from a sugar oxazo-line.²⁵ But the synthesis of the disaccharide N,N′-diacetylchitobiose (172 mM) from GlcNAc (3-fold excess) and GlcNAc-oxazoline by a chitinase from Bacillus sp. suffered from low yield (43%). Synthesis of tri- and tetrasaccharides from disaccharide oxazolines was also de-scribed.²⁶ However, significantly lower yields (50-70%, based on donor) and product concentrations (-10-fold) were obtained, compared to the transglycosylase process presented herein.²⁶ Typically, polyaddition of sugar oxazoline derivatives or step-wise synthesis of oligosaccharide -containing macromonomers was reported for chitinases.^{26, 46}

In a preferred embodiment, in the method for producing lacto-N-tetraose described above, the lacto-N-biosidase has an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 3 (LnbB, GenBank accession number: EU281545.1) and carries a mutation at a position counted as position 320 of SEQ ID NO: 3 and/or at a position counted as position 419 of SEQ ID NO: 3, preferably carries a glutamic acid or an alanine at a position counted as position 320 of SEQ ID NO: 3 and/or carries a phenylalanine at a position counted as position 419 of SEQ ID NO: 3, or the lacto-N-biosidase comprises an amino acid sequence of any of SEQ ID NOs: 9 to 11 or comprises an amino acid sequence having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to any of SEQ ID NOs: 9 to 11.

SEQ ID NOs: 9 to 11 represent the truncated versions of LnbB carrying the above described mutations. The mutations at position 320 and 419 of SEQ ID NO: 3 are not to be understood as absolute, but corresponding mutations at equivalent positions of enzymes belonging to the GH20 family or to the EC class EC 3.2.1.140 and having a glycosidase activity, in particular lacto-N-biosidases, e.g. homolog enzymes from other organisms, also provide the functionality according to the disclosure. Equivalent positions can be identified by a global sequence alignment as shown in FIG. 16.

Preferably, the LnbB used in the context of the present disclosure has a non-naturally occurring amino acid sequence and represents an artificial construct, which has been taken out of its natural biological context and has been specifically designed for the purpose as described herein. It may therefore be a truncated, but functional version of the naturally occurring protein and may carry certain modifications, for example those that simplify its biotechnological production or purification, e.g. a His-tag.

The disclosure also provides nucleotide sequences, in particular DNA sequences, and methods as described above making use of such nucleotide sequences, wherein the nucleotide sequences hybridize under stringent conditions with a DNA or RNA sequence, which codes for an enzyme of the glycoside hydrolase family GH20 as described above, in particular a lacto-N-biosidase carrying a mutation at a position counted as position 320 of SEQ ID NO: 3 and/or at a position counted as position 419 of SEQ ID NO: 3, preferably carrying a glutamic acid or an alanine at a position counted as position 320 of SEQ ID NO: 3 and/or carrying a phenylalanine at a position counted as position 419 of SEQ ID NO: 3 or a truncated functional version of the enzyme according to any of SEQ ID NO: 9 to 11.

The trans-glycosylation activities of wild-type LnbB and the variants thereof were assayed under the same reaction conditions as described above for the Bbhl. Full reaction time courses of LNT synthesis from LNB-oxazoline or LNB-β-pNP onto lactose (30-50-fold excess) are depicted in FIG. 6 (LNB-oxazoline) and the FIG. 7 (LNB-β-pNP). HPLC analysis was used for quantification (FIG. 8). Further confirmation of product identity was obtained by TLC analysis (FIG. 9). Table 2 compares the specific activities of the LnbB variants to that of the wild-type enzyme for both donor substrates. Similar results were obtained with LNB-oxazoline and LNB-β-pNP, but LNB-oxazoline was the preferred substrate. As observed for Bbhl, replacement in SEQ ID NO: 3 of the corresponding Asp³²⁶ by Glu in LnbB was the most beneficial mutation. In contrast to wild-type LnbB, which showed strong product hydrolysis, secondary hydrolysis was completely abolished by this mutation. LNT was obtained from LNB-oxazoline in a yield of 30% (3.5 mM), and no product hydrolysis was detected within 22 h. Incubation with a 5-fold increased amount of D320E gave the same product yield correspondingly faster (FIG. 10).

TABLE 2
Activity and selectivity parameters of wild-type and site-directed
variants of LnbB

| | LNB-oxazoline^{a} | | LNB-β-pNP^{b} | |
|---|---|---|---|---|
| | Trans- | Secondary | Trans- | Secondary |
| | glycosylation | hydrolysis | glycosylation | hydrolysis |
| LnbB | (U mg⁻¹) | (U mg⁻¹) | (U mg⁻¹) | (U mg⁻¹) |
| WT | 25 | 9.5 × 10⁻¹ | 4.0 | 1.0 |
| D320E | 2.2 × 10⁻¹ | n.d. | 2.1 × 10⁻¹ | 2.3 × 10⁻⁴ |
| D320A | 8.9 × 10⁻⁴ | n.d. | 8.0 × 10⁻⁴ | n.d. |
| Y419F | 7.5 × 10⁻² | 4.8 × 10⁻³ | 3.3 × 10⁻² | 3.8 × 10⁻² |

n.d., not detectable.
^{a}12 mM LNB-oxazoline, 600 mM lactose, 37° C., pH 7.5.
^{b}20 mM LNB-β-pNP, 600 mM lactose, 15% DMSO, 37° C., pH 5.8.

So far, the LnbB was identified as critical enzyme for the degradation of HMOs, liberating LNB from their non-reducing end.^{6,28,29} But the wild-type enzyme was not a useful catalyst for the reverse reaction, the trans-glycosylation.⁶ Efficient and practical enzymatic synthesis of LNT has not been achieved. A Bacillus circulans β-galactosidase gave LNT in 19% yield, together with an undesired LNT regioisomer.¹⁶ Conversion of lactose into LNT by lacto-N-bio-sidase from Aureobacterium sp. L-101 was very inefficient (yield <4%).¹⁶ LNT benzyl glycoside was produced from LNT II benzyl glycoside (synthesized from lactose benzyl glycoside and UDP-GlcNAc by an N-acetylglucosaminyltransferase) and UDP-Gal in 74% yield (˜10 mM), using a GST-tagged E. coli β1,3-galactosyltransferase fusion protein.³⁰ However, expensive nucleotide -activated sugars were needed. Large-scale production of LNT (173 g, 12.7 g L⁻¹) was only achieved by fed-batch cultivation of metabolically engineered E. coli, overexpressing tailored glycosyltransferases.¹⁷ However, a one to one mixture of LNT and LNT II was obtained.¹⁷

In the context of the present disclosure, the transglycosylase mutant D320E was created. Synthesis of LNT from LNB-oxazoline without any detectable hydrolysis of the product was demonstrated.

In one preferred embodiment, the methods described above further comprise the step:

(ii) deactivating the enzyme of the glycoside hydrolase family 20 (GH20).

Deactivation of the enzyme of the glycoside hydrolase family 20 (GH20) allows further processing of the reaction product(s) obtained in the method according to the disclosure, without risking unwanted side reactions. For example, it is possible to further react lacto-N-tri-ose II to lacto-N-tetraose or lacto-N-neotetraose. For example, β-galactosidases^{16,31} or Leloir glycosyltransferases^{19, 20, 21} can be used, giving lacto-N-neotetraose (LNnT) or lacto-N-tetraose (LNT).

In a further preferred embodiment, the method for producing lacto-N-triose II described above therefore further comprises the step:

(iii) adding a β-galactosidase or a galactosyl transferase and UDP-galactose, to the mixture of step (i) or, preferably to the mixture of step (ii), and optionally adding further lactose to obtain lacto-N-tetraose or lacto-N-neotetraose.

Further reaction of lacto-N-triose II with a β-galactosidase provides LNT or LNnT by regioselective galactosyl-transfer.^{16,31} Likewise, reaction of lacto-N-triose II with a galactosyl transferase and UDP-galactose as a donor substrate provides LNT or LNnT.^{19, 20, 21} Alternatively, UDP-glucose can be combined with an epimerase to provide the UDP-galactose donor substrate in situ.

The present disclosure also relates to the use of an enzyme of the glycoside hydrolase family 20 (GH20) according to the classification of the Carbohydrate-Active-Enzymes (CAZy) database (http://www.cazy.org, updated on 20 Nov. 2018) for producing lacto-N-triose II or lacto-N-tetraose from lactose.

Preferred is the use of an enzyme of the glycoside hydrolase family 20 (GH20) for producing lacto-N-triose II as described above, wherein the enzyme of the glycoside hydrolase family 20 (GH20) is 8-N-acetylhexosaminidase, preferably β-N-acetylhexosaminidase of Bifidobacterium bifidum JCM1254 having an amino acid sequence of SEQ ID NO: 1 (Bbhl, GenBank accession number: AB504521.1) or an enzyme having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 1 and having β-N-acetylhexosaminidase activity, or a β-N-acetylhexosaminidase enzyme comprising an amino acid sequence of SEQ ID NO: 2, or a β-N-acetylhexosaminidase enzyme comprising an amino acid sequence having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 2.

Particularly preferred is the use of an enzyme of the glycoside hydrolase family 20 (GH20) for producing lacto-N-triose II as described above, wherein the β-N-acetylhexosaminidase has an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 1 (Bbhl, GenBank accession number: AB504521.1) and carries a mutation at a position counted as position 746 of SEQ ID NO: 1 and/or at a position counted as position 827 of SEQ ID NO: 1, preferably carries a glutamic acid or an alanine or a glutamine at a position counted as position 746 of SEQ ID NO: 1 and/or carries a phenylalanine at a position counted as position 827 of SEQ ID NO: 1, or wherein the β-N-acetylhexosaminidase comprises an amino acid sequence of any of SEQ ID NOs: 5 to 8 or comprises an amino acid sequence having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to any of SEQ ID NOs: 5 to 8.

Preferably, the Bbhl used in the context of the present disclosure has a non-naturally occurring amino acid sequence and represents artificial constructs, which has been taken out of its natural biological context and has been specifically designed for the purpose as described herein. It may therefore be a truncated, but functional version of the naturally occurring protein and may carry certain modifications, for example those that simplify its biotechnological production or purification, e.g. a His-tag.

Further preferred is the use of an enzyme of the glycoside hydrolase family 20 (GH20) for producing lacto-N-tetraose as described above, wherein the enzyme of the glycoside hydrolase family 20 (GH20) is lacto-N-biosidase, preferably lacto-N-biosidase of Bifidobacterium bifidum JCM1254 having an amino acid sequence of SEQ ID NO: 3 (LnbB, GenBank accession number: EU281545.1), or an enzyme having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 3 and having lacto-N-biosidase activity, or a lacto-N-bio-sidase enzyme comprising an amino acid sequence of SEQ ID NO: 4, or a lacto-N-biosidase enzyme comprising an amino acid sequence having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 4.

Particularly preferred is the use of an enzyme of the glycoside hydrolase family 20 (GH20) for producing lacto-N-tetraose as described above, wherein the lacto-N-biosidase has an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 3 (LnbB, Gen-Bank accession number: EU281545.1) and carries a mutation at a position counted as position 320 of SEQ ID NO: 3 and/or at a position counted as position 419 of SEQ ID NO: 3, preferably carries a glutamic acid or an alanine at a position counted as position 320 of SEQ ID NO: 3 and/or carries a phenylalanine at a position counted as position 419 of SEQ ID NO: 3, or wherein the lacto-N-biosidase comprises an amino acid sequence of any of SEQ ID NOs: 9 to 11 or comprises an amino acid sequence having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to any of SEQ ID NOs: 9 to 11.

Preferably, the LnbB used in the context of the present disclosure has a non-naturally occurring amino acid sequence and represents an artificial construct, which has been taken out of its natural biological context and has been specifically designed for the purpose as described herein. It may therefore be a truncated, but functional version of the naturally occurring protein and may carry certain modifications, for example those that simplify its biotechnological production or purification, e.g. a His-tag.

According to a further preferred embodiment of the use as described above, the enzyme of the glycoside hydrolase family 20 (GH20) is used in a method according to any of the embodiments described above.

The present disclosure describes a highly productive transglycosylation process, which is fit for process scale-up to enable production at demonstration scale. The current LNT II synthesis is performance-wise without precedent in preparation of HMO core-structures by enzymatic glycosylation. Significant intensification of biocatalysis compared to β-N-acetylhex-osaminidase - and glycosyltransferase-catalyzed reactions, respectively, was achieved using the enzymes described above.^{5, 16, 17, 18, 19, 20, 21, 22, 23} The simple recovery of LNT II in a purity of about 80% with mostly lactose as impurity is clearly beneficial for the scalability of the established process. A transglycosylation process allowing actual production in bulk quantities is novel and broadly relevant in the field. LNT II is one of the major building blocks of HMOs.³² The simple and sophisticated LNT II synthesis described herein offers the entry to HMO structures. In addition, LNT and LNnT can be obtained using the enyzmes described above.

In the context if the present disclosure, certain mutants of Bbhl and LnbB have been identified, which are highly useful in the production of HMO core structures.

The present disclosure therefore also relates to β-N-acetylhexosaminidase having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 1 (Bbhl, Gen-Bank accession number: AB504521.1) and carrying a mutation at a position counted as position 746 of SEQ ID NO: 1 and/or at a position counted as position 827 of SEQ ID NO: 1, preferably carrying a glutamic acid or an alanine or a glutamine at a position counted as position 746 of SEQ ID NO: 1 and/or carrying a phenylalanine at a position counted as position 827 of SEQ ID NO: 1, or β-N-acetylhexosaminidase comprising an amino acid sequence of any of SEQ ID NOs: 5 to 8 or comprising an amino acid sequence having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to any of SEQ ID NOs: 5 to 8.

Preferably, the Bbhl according to the present disclosure has a non-naturally occurring amino acid sequence and represents an artificial construct, which has been taken out of its natural biological context and has been specifically designed for the purpose as described herein. It may therefore be a truncated, but functional version of the naturally occurring protein and may carry certain modifications, for example those that simplify its biotechnological production or purification, e.g. a His-tag.

Furthermore, the present disclosure also relates to lacto-N-biosidase having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 3 (LnbB, GenBank accession number: EU281545.1) and carrying a mutation at a position counted as position 320 of SEQ ID NO: 3 and/or at a position counted as position 419 of SEQ ID NO: 3, preferably carrying a glutamic acid or an alanine at a position counted as position 320 of SEQ ID NO: 3 and/or carrying a phenylalanine at a position counted as position 419 of SEQ ID NO: 3, or lacto-N -biosidase comprising an amino acid sequence of any of SEQ ID NOs: 9 to 11 or comprising an amino acid sequence having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to any of SEQ ID NOs: 9 to 11.

Preferably, the LnbB according to the present disclosure has a non-naturally occurring amino acid sequence and represents artificial constructs, which has been taken out of its natural biological context and has been specifically designed for the purpose as described herein. It may therefore be a truncated, but functional version of the naturally occurring protein and may carry certain modifications, for example those that simplify its biotechnological production or purification, e.g. a His-tag.

The disclosure also provides nucleotide sequences, in particular DNA sequences, wherein the nucleotide sequences hybridize under stringent conditions with a DNA or RNA sequence, which codes for an enzyme of the glycoside hydrolase family GH20 as described above, in particular a β-N-acetyl hexosam n da se carrying a mutation at a position counted as position 746 of SEQ ID NO: 1 and/or at a position counted as position 827 of SEQ ID NO: 1, preferably carrying a glutamic acid or an alanine or a glutamine at a position counted as position 746 of SEQ ID NO: 1 and/or carrying a phenylalanine at a position counted as position 827 of SEQ ID NO: 1 or a lacto-N-biosidase carrying a mutation at a position counted as position 320 of SEQ ID NO: 3 and/or at a position counted as position 419 of SEQ ID NO: 3, preferably carrying a glutamic acid or an alanine at a position counted as position 320 of SEQ ID NO: 3 and/or carrying a phenylalanine at a position counted as position 419 of SEQ ID NO: 3 or comprising an amino acid sequence according to any of SEQ ID NO: 5 to 11.

The present disclosure also provides an aqueous solution comprising an enzyme of the glycoside hydrolase family 20 (GH20) as defined above, lactose, preferably at least 50 g/L lactose, more preferably at least 100 g/L lactose, particularly preferably at least 150 g/L lactose, most preferably at least 190 g/L lactose and
a) glucosamine-oxazoline and/or lacto-N-biose-oxazoline; and/or
b) lacto-N-triose II and/or lacto-N-tetraose.
   Preferably, the aqueous solution is free or essentially free of organic solvents.

“Essentially free” in this context preferably means that there is 10 g/L or less, more preferably 1 g/L or less of an organic solvent present in the solution.

In a preferred embodiment, the enzyme of the glycoside hydrolase family 20 (GH20) is a β-N-acetylhexosaminidase, preferably β-N-acetylhexosaminidase of Bifidobacterium bifidum JCM1254 having an amino acid sequence of SEQ ID NO: 1 (Bbhl, GenBank accession number: AB504521.1) or an enzyme having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 1 and having β-N-acetylhexosaminidase activity, or a β-N-acetlIhex-osaminidase enzyme comprising an amino acid sequence of SEQ ID NO: 2, or a β-N-acetylhex-osaminidase enzyme comprising an amino acid sequence having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 2.

In a further preferred embodiment, the enzyme of the glycoside hydrolase family 20 (GH20) is lacto-N-biosidase, preferably lacto-N-biosidase of Bifidobacterium bifidum JCM1254 having an amino acid sequence of SEQ ID NO: 3 (LnbB, GenBank accession number: EU281545.1), or an enzyme having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 3 and having lacto-N-biosidase activity, or a lacto-N-biosidase enzyme comprising an amino acid sequence of SEQ ID NO: 4, or a lacto-N-biosidase enzyme comprising an amino acid sequence having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 4.

Preferably, the enzyme(s) present in the aqueous solution have a non-naturally occurring amino acid sequence and represent artificial constructs, which have been taken out of their natural biological context and have been specifically designed for the purpose as described herein. They may therefore be truncated, but functional versions of the naturally occurring proteins and may carry certain modifications, for example those that simplify their biotechnological production or purification, e.g. a His-tag.

Particularly preferred is an aqueous solution as described above, wherein the p-N-acetylhexosaminidase has an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 1 (Bbhl, GenBank accession number: AB504521.1) and carries a mutation at a position counted as position 746 of SEQ ID NO: 1 and/or at a position counted as position 827 of SEQ ID NO: 1, preferably carries a glutamic acid or an alanine or a glutamine at a position counted as position 746 of SEQ ID NO: 1 and/or carries a phenylalanine at a position counted as position 827 of SEQ ID NO: 1, or wherein the β-N-acetylhexosaminidase comprises an amino acid sequence of any of SEQ ID NOs: 5 to 8 or comprises an amino acid sequence having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to any of SEQ ID NOs: 5 to 8.

Further particularly preferred is an aqueous solution as described above, wherein the lacto-N-biosidase has an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to SEQ ID NO: 3 (LnbB, GenBank accession number: EU281545.1) and carries a mutation at a position counted as position 320 o SEQ ID NO: 3 and/or at a position counted as position 419 of SEQ ID NO: 3, preferably carries a glutamic acid or an alanine at a position counted as position 320 of SEQ ID NO: 3 and/or carries a phenylalanine at a position counted as position 419 of SEQ ID NO: 3, or wherein the lacto-N-biosidase comprises an amino acid sequence of any of SEQ ID NOs: 9 to 11 or comprises an amino acid sequence having an amino acid sequence identity of at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% to any of SEQ ID NOs: 9 to 11.

### Embodiments

Method for producing lacto-N-triose II and/or lacto-N-tetraose comprising the step:
(i) reacting glucosamine-oxazoline and/or lacto-N-biose-oxazoline with lactose cata-lysed by an enzyme of the glycoside hydrolase family 20 (GH20) according to the classification of the Carbohydrate-Active-Enzymes (CAZy) database (http://www.cazy.org, updated on 20 Nov. 2018) to obtain lacto-N-triose II and/or lacto-N-tetraose.

2. Method according to embodiment 1, wherein step (i) is performed in an aqueous solution containing at least 50 g/L lactose, preferably at least 100 g/L lactose, particularly preferably at least 150 g/L lactose, most preferably at least 190 g/L lactose.

3. Method according to embodiment 1 or embodiment 2, wherein the method is performed under conditions that are free or essentially free of organic solvents.

4. Method according to any one of embodiments 1 to 3, wherein the glucosamin -oxazoline and/or lacto-N-biose-oxazoline, respectively, is added to the lactose and the enzyme of the glycoside hydrolase family 20 (GH20) over a period of at least 20 minutes, preferably at least 60 minutes.

5. Method for producing lacto-N-triose II according to any one of embodiments 1 to 4, wherein the enzyme of the glycoside hydrolase family 20 (GH20) is a β-N-acetylhex-osamnidase, preferably β-N-acetylhexosamndase of Bifidobacterium bifidum JCM1254 having an amino acid sequence of SEQ ID NO: 1 (Bbhl, GenBank accession number: AB504521.1) or an enzyme having an amino acid sequence identity of at least 70% to SEQ ID NO: 1 and having β-N-a cetylhexosaminidase activity, or a β-N-a cetylhexosaminidase enzyme comprising an amino acid sequence of SEQ ID NO: 2, or a β-N-acetylhexosaminidase enzyme comprising an amino acid sequence having an amino acid sequence identity of at least 70% to SEQ ID NO: 2.

6. Method for producing lacto-N-tetraose according to any one of embodiments 1 to 4, wherein the enzyme of the glycoside hydrolase family 20 (GH20) is a lacto-N-biosidase, preferably lacto-N-biosidase of Bifidobacterium bifidum JCM1254 having an amino acid sequence of SEQ ID NO: 3 (LnbB, GenBank accession number: EU281545.1), or an enzyme having an amino acid sequence identity of at least 70% to SEQ ID NO: 3 and having lacto-N-biosidase activity, or a lacto-N-biosidase enzyme comprising an amino acid sequence of SEQ ID NO: 4, or a lacto-N-biosidase enzyme comprising an amino acid sequence having an amino acid sequence identity of at least 70% to SEQ ID NO: 4.

7. Method according to any of the preceding embodiments, wherein the method further comprises the step:
(ii) deactivating the enzyme of the glycoside hydrolase family 20 (GH20).

8. Method for producing lacto-N-triose II according to any of embodiments 1 to 5 or 7 further comprising the step:
(iii) adding a β-galactosidase or a galactosyl transferase and UDP-galactose, to the mixture of step (i) or, preferably to the mixture of step (ii), and optionally adding further lactose to obtain lacto-N-tetraose or lacto-N-neotetraose.

9. Use of an enzyme of the glycoside hydrolase family 20 (GH20) according to the classification of the Carbohydrate-Active-Enzymes (CAZy) database (http://www.cazy.org, updated on 20 Nov. 2018) for producing lacto-N-triose II or lacto-N-tetraose from lactose, preferably use of an enzyme as defined in embodiment 5 for producing lacto-N-triose II or use of an enzyme as defined in embodiment 6 for producing lacto-N-tetraose.

10. Use according to embodiment 9, wherein the enzyme of the glycoside hydrolase family 20 (GH20) is used in a method according to any of embodiments 1 to 8.

11. β-N-acetylhexosaminidase having an amino acid sequence identity of at least 70% to SEQ ID NO: 1 (Bbhl, GenBank accession number: AB504521.1) and carrying a mutation at a position counted as position 746 of SEQ ID NO: 1 and/or at a position counted as position 827 of SEQ ID NO: 1, preferably carrying a glutamic acid or an alanine or a glutamine at a position counted as position 746 of SEQ ID NO: 1 and/or carrying a phenylalanine at a position counted as position 827 of SEQ ID NO: 1, or β-N-acetylhexosaminidase comprising an amino acid sequence of any of SEQ ID NOs: 5 to 8 or comprising an amino acid sequence having an amino acid sequence identity of at least 70% to any of SEQ ID NOs: 5 to 8.

12. Lacto-N-biosidase having an amino acid sequence identity of at least 70% to SEQ ID NO: 3 (LnbB, GenBank accession number: EU281545.1) and carrying a mutation at a position counted as position 320 of SEQ ID NO: 3 and/or at a position counted as position 419 of SEQ ID NO: 3, preferably carrying a glutamic acid or an alanine at a position counted as position 320 of SEQ ID NO: 3 and/or carrying a phenylalanine at a position counted as position 419 of SEQ ID NO: 3, or lacto-N-biosidase comprising an amino acid sequence of any of SEQ ID NOs: 9 to 11 or comprising an amino acid sequence having an amino acid sequence identity of at least 70% to any of SEQ ID NOs: 9 to 11.

13. Aqueous solution comprising an enzyme of the glycoside hydrolase family 20 (GH20) as defined in any of the preceding embodiments, lactose, preferably at least 50 g/L lactose, more preferably at least 100 g/L lactose, particularly preferably at least 150 g/L lactose, most preferably at least 190 g/L lactose and
a) glucosamine-oxazoline and/or lacto-N-biose-oxazoline; and/or
b) lacto-N-triose II and/or lacto-N-tetraose.

### BRIEF DESCRIPTION OF THE DRAWINGS

FIG. 1: LNT II synthesis by wild-type Bbhl and mutants thereof. Time courses show synthesis from 60 mM GlcNAc-oxazoline using a 10-fold excess of lactose. (a) Wild-type, 0.23 μM; (b) D746E, 8.4 μM; (c) Y827F, 4 μM; (d) D746A, 18 μM; (e) D746Q, 9 pM. LNT II, filled circles; yield, open circles. (f) Trans-glycosylation versus primary hydrolysis. RTH is the ratio of the specific trans-glycosylation activity of GlcNAc-to-lactose transfer to the specific activity of non-productive GlcNAc-oxazoline hydrolysis (primary hydrolysis) under ‘synthesis conditions’. For the wild type and each mutant three bars are shown, the left bar indicates the specific trans-glycosylation activity; the middle bar indicates the maximum LNT II yield; the right bar indicates the RTH value.

FIG. 2: LNT II synthesis from GlcNAc-p-pNP by wild-type Bbhl and mutants thereof. Time courses show synthesis from 20 mM GlcNAc-β-pNP using a 20-fold excess of lactose in the presence of 20% DMSO. (a) Wild-type, 0.12 μM; (b) D746E, 8.4 μM; (c) D746A, 24 μM; (d) D746Q, 8.6 μM; (e) Y827F, 8 μM. LNT II, filled circles; yield, open circles; pNP released, open triangles. (f) TLC-analysis of LNT II synthesis using Bbhl enzymes. Reaction mixtures were 2-fold diluted. Carbohydrates were visualized by thymol-sulfuric acid reagent. pNP was detected by UV (254 nm). Lane 1, pNP; lane 2, lactose; lane 3, LNT II; lane 4, wild-type reaction after 1.5 h; lane 5, D746E reaction after 20 min; lane 6, Y975F reaction after 1.5 h; lane 7, D746A reaction after 8 h; lane 8, D746Q reaction after 6 h.

FIG. 3: HPLC analysis of LNT II synthesis from GlcNAc-oxazoline by wild-type Bbhl and mutants thereof. Overlay of HPLC-chromatograms showing maximum LNT II formation obtained by various enzyme variants of Bbhl. Reaction mixtures contained 60 mM GlcNAc-oxazoline, a 10-fold excess of lactose and 0.23-18 μM of the enzymes. Samples were 10-fold (D746Q) and 25-fold diluted (all other enzymes), respectively. UV-detection at 195 nm was used.

FIG. 4: TLC analysis of LNT II synthesis from GlcNAc-oxazoline by wild-type Bbhl and mutants thereof. 130 mM (D746E) or 60 mM (all other enzymes) GlcNAc-oxazoline, 600 mM lactose and 0.23-18 μM of the enzymes were used. Carbohydrates were visualized by thymol-sulfuric acid reagent. GlcNAc was also detected by UV (254 nm) (a) Lane 1, GlcNAc; lane 2, lactose; lane 3, LNT II; lane 4, wild-type reaction after 1 h, 1:10; lane 5, D746E reaction after 50 min, 1:50; lane 6, D746A reaction after 5 h, 1:25; lane 7, galactose; lane 8, glucose. (b) Lane 1, GlcNAc; lane 2, lactose; lane 3, LNT II; lane 4, Y827F reaction after 50 min, 1:9.

FIG. 5: LNT II synthesis from GlcNAc-oxazoline by wild-type Bbhl and the D746E mutant. Time courses show synthesis from 64 mM GlcNAc-oxazoline and 20 mM lactose. (a) Wild-type, 0.23 μM; (b) D746E, 8.4 μM. LNT II, filled circles; yield, open circles.

FIG. 6: LNT synthesis by wild-type LnbB and mutants thereof. Time courses show synthesis from 12 mM LNB-oxazoline using a 50-fold excess of lactose. (a) Wild-type, 0.5 μM; (b) D320E, 4 μM; (c) D320A, 20 μM; (d) Y419F, 4 μM. LNT , filled circles; yield, open circles; LNT II, filled triangles.

FIG. 7: LNT synthesis from LNB-β-pNP by wild-type LnbB and mutants thereof. Time courses show synthesis from 20 mM LNB-β-pNP in the presence of 15% DMSO, using a 30-fold excess of lactose. (a) Wild-type, 0.5 μM; (b) D320E, 10 μM; (c) D320A, 20 μM; (d) Y419F, 4 μμM. LNT, filled circles; yield, open circles; pNP released, open triangles. (e) TLC-analysis of LNT synthesis using LnbB enzymes. Reaction mixtures were 5-fold diluted. Carbohydrates were visualized by thymol-sulfuric acid reagent. Lane 1, LNB; lane 2, lactose; lane 3, LNT; lane 4, wild-type reaction after 10 min; lane 5, D320E reaction after 20 min; lane 6, Y419F reaction after 2.5 h; lane 7, D320A reaction after 21 h.

FIG. 8: HPLC analysis of LNT synthesis from LNB-oxazoline by wild-type LnbB and mutants thereof. Overlay of HPLC-chromatograms showing maximum LNT formation obtained by various enzyme variants of LnbB. Reaction mixtures contained 12 mM LNB-oxazoline, a 50-fold excess of lactose and 0.5-20 μM of the enzymes. Samples were 5-fold diluted. UV-detection at 195 nm was used.

FIG. 9: TLC analysis of LNT synthesis from LNB-oxazoline by wild-type LnbB and mutants thereof. 12 mM LNB-oxazoline, a 50-fold excess of lactose and 0.5-20 pM of the enzymes were used. Reaction mixtures were 5-fold diluted. Carbohydrates were visu-alized by thymol-sulfuric acid reagent. (a) Lane 1, GlcNAc; lane 2, LNB; lane 3, lactose; lane 4, LNT II; lane 5, LNT; lane 6, wild-type reaction after 10 min; lane 7, D320E reaction after 20 min; lane 8, Y419F reaction after 2.5 h; lane 9, D320A reaction after 21 h. (b) Lane 1, GlcNAc; lane 2, LNB; lane 3, LNT II; lane 4, LNT; lane 5, wild-type reaction after 0 min; lane 6, wild-type reaction after 10 min; lane 7, D320E reaction after 0 h; lane 8, D320E reaction after 1 h; lane 9, lactose.

FIG. 10: Synthesis of LNT by the LnbB D320E mutant. Reaction mixture contained 12 mM LNB-oxazoline, a 50-fold excess of lactose and 20 μM D320E. LNT , filled circles; yield, open circles; LNT II, filled triangles.

FIG. 11: Bulk synthesis of LNT II by the Bbhl D746E mutant. (a) Time-course of LNT II synthesis by the D746E variant (4 pM) using equimolar amounts of GlcNAc-oxazoline and lactose (600 mM). (b) Comparison of wild-type Bbhl (triangles) and D746E mutant (circles) for LNT2 synthesis, using a 2.4 fold excess of lactose over GlcNAc-oxazoline (255 mM) and 0.4 pM of enzyme. LNT II, filled symbols; yield, open symbols. Overlay of HPLC-UV traces (c) and HPLC-RI traces (d) used to evaluate the purity of LNT II produced on gram-scale. Note, the first peak in the HPLC-RI traces is the injection peak.

FIG. 12: LNT II synthesis with increasing GlcNAc-oxazoline concentration by the D746E mutant of Bbhl. Time courses show synthesis using varying concentrations of GlcNAc-oxazoline, 600 mM lactose and 4.2 μM D746E. GlcNAc-oxazoline concentration was: (a) 130 mM, (b) 260 mM, (c) 500 mM. LNT II, filled circles; yield, open circles. (d) TLC-analysis of LNT II using 500 mM of GlcNAc-oxazoline. Reaction mixtures were 50-fold diluted. Lane 1, GlcNAc; lane 2, lactose; lane 3, LNT II; lane 4, 0 h; lane 5, 2.5 min; lane 6, 30 min; lane 7, 5 h; lane 8, 23.5 h. (e) TLC-analysis of LNT II using 600 mM of GlcNAc-oxazoline. Reaction mixtures were 200-fold diluted. Lane 1, GlcNAc; lane 2, lactose; lane 3, LNT II; lane 4, 0 h; lane 5, 2.5 min; lane 6, 5 min; lane 7, 30 min; lane 8, 7 h; lane 9, 23.5 h. Carbohydrates were visualized by thymol-sulfuric acid reagent. GlcNAc was also detected by UV (254 nm).

FIG. 13: LNT II synthesis from GlcNAc-β-pNP by wild-type Bbhl and the D746E mutant. Time courses show synthesis from 100 mM GlcNAc-β-pNP and 600 mM lactose in the presence of 20% DMSO. (a) D746E, 8.4 μM; (b) wild-type, 0.12 μM. LNT II, filled circles; yield, open circles; pNP released, open triangles.

FIG. 14: ¹H NMR spectrum of isolated LNT II. LNT II was dissolved in D₂O. Spectrum is in accordance with previously published data.⁴

FIG. 15: ¹³C NMR spectrum of isolated LNT II. LNT II was dissolved in D₂O. Full spectrum and partial spectrum (inset) showing that LNT II (GlcNAc-β1,3-Gal-β1,4-Glc) was the only regioisomer formed (82 ppm). No other regioisomers could be detected. Spectrum is in accordance with previously published data.⁴

FIG. 16: Global sequence alignment of the wildtype and mutant constructs of Bbhl and LnbB (SEQ ID NOs: 12-20) and corresponding sequences from other organ-isms (SEQ ID NOs 21-24). The alignment was performed with the program “MegAlign Pro” Version: 12.2.0 (82) Copyright © 2012-2015, DNASTAR, Inc.. The used algorithm was “MUSCLE” (Multiple Sequence Comparison by Log-Expectation). The aligned sequences are the following: Bbhl, wild type (wt), truncated construct including a His-tag (SEQ ID NO: 12), Bbhl, D746E mutant, truncated construct including a His-tag (SEQ ID NO: 13), Bbhl, D746A mutant, truncated construct including a His-tag (SEQ ID NO: 14), Bbhl, D746Q mutant, truncated construct including a His-tag (SEQ ID NO: 15), Bbhl, Y827F mutant, truncated construct including a His-tag (SEQ ID NO: 16), LnbB, wild type (wt), truncated construct including a His-tag (SEQ ID NO: 17), LnbB, D320E mutant, truncated construct including a His-tag (SEQ ID NO: 18), LnbB, D320A mutant, truncated construct including a His-tag (SEQ ID NO: 19), LnbB, Y419F mutant, truncated construct including a His-tag (SEQ ID NO: 20); Hex 1, from Actinomycetales bacterium, GenBank AKC34128.1 (SEQ ID NO: 21), Hex 2, from Bacteroidetes bacterium, GenBank AKC34129.1 (SEQ ID NO: 22), Chb, from Serratia marcescens, GenBank AAB03808.1 (SEQ ID NO: 23), SpHex, from Streptomyces plicatus, GenBank AAC38798.3 (SEQ ID NO: 24). The consensus sequence of the alignment is also shown (SEQ ID NO: 25).

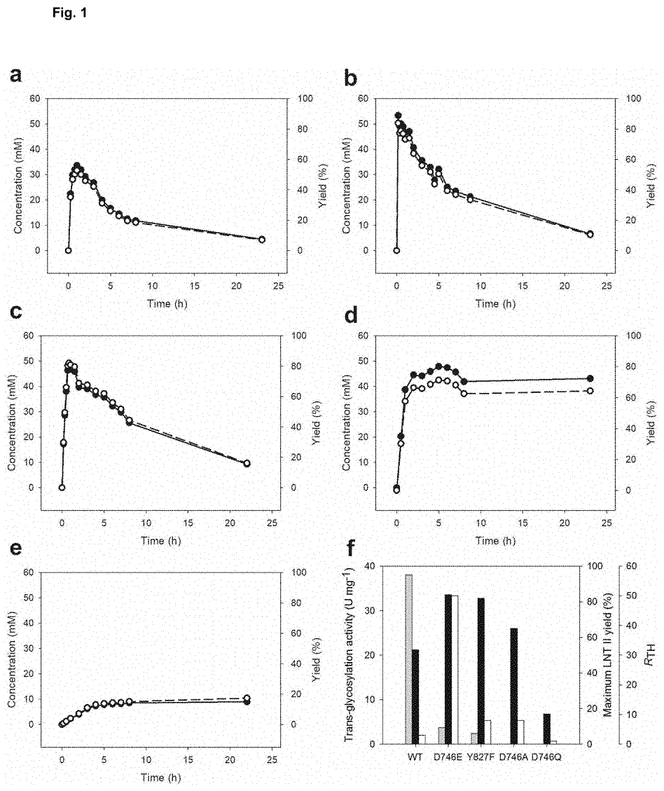

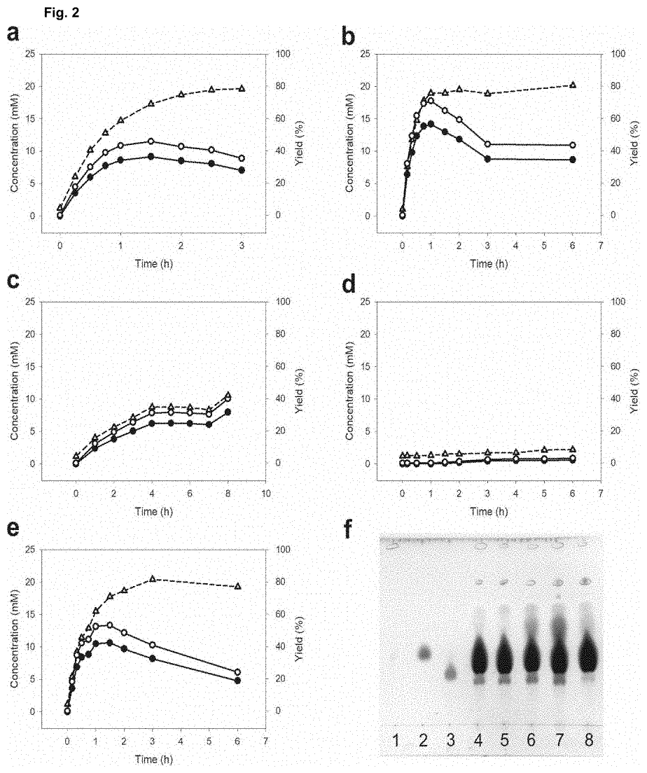

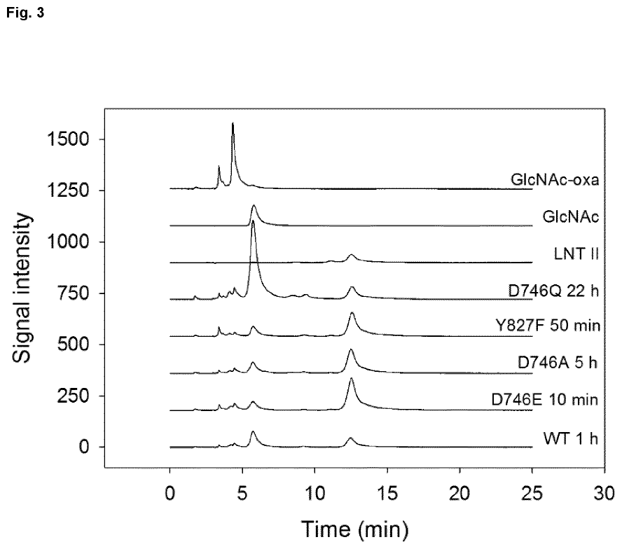

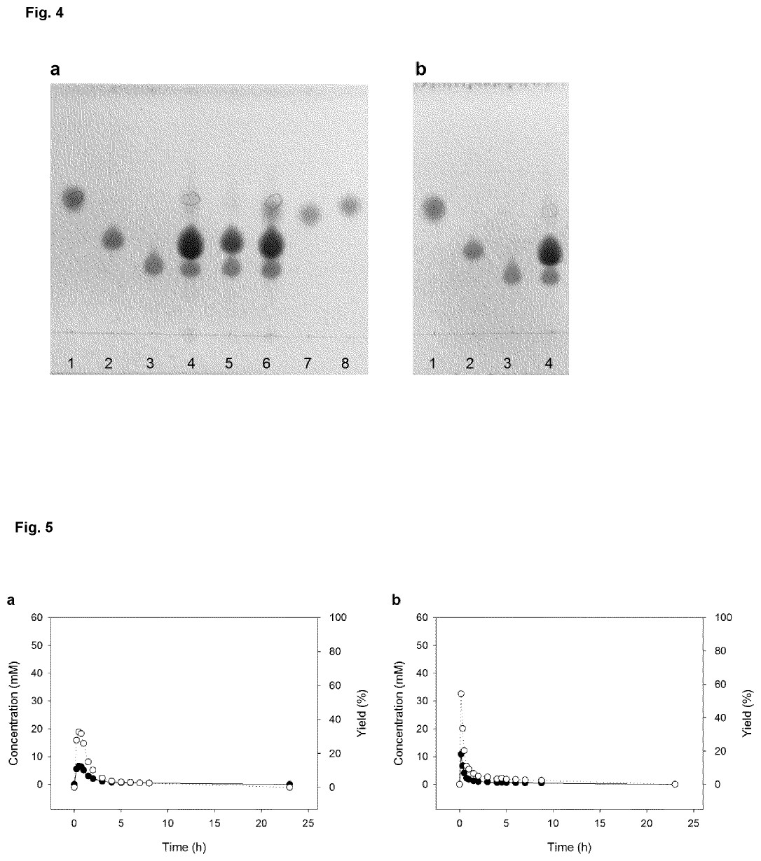

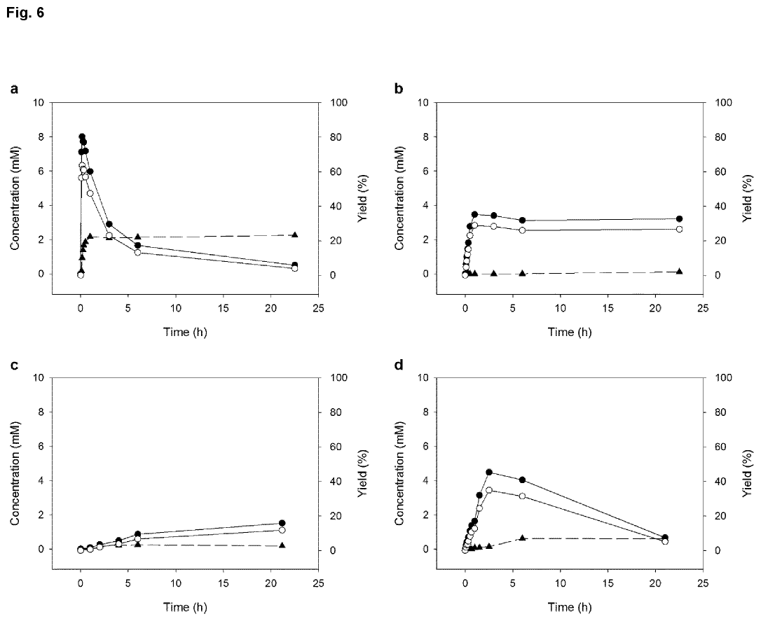

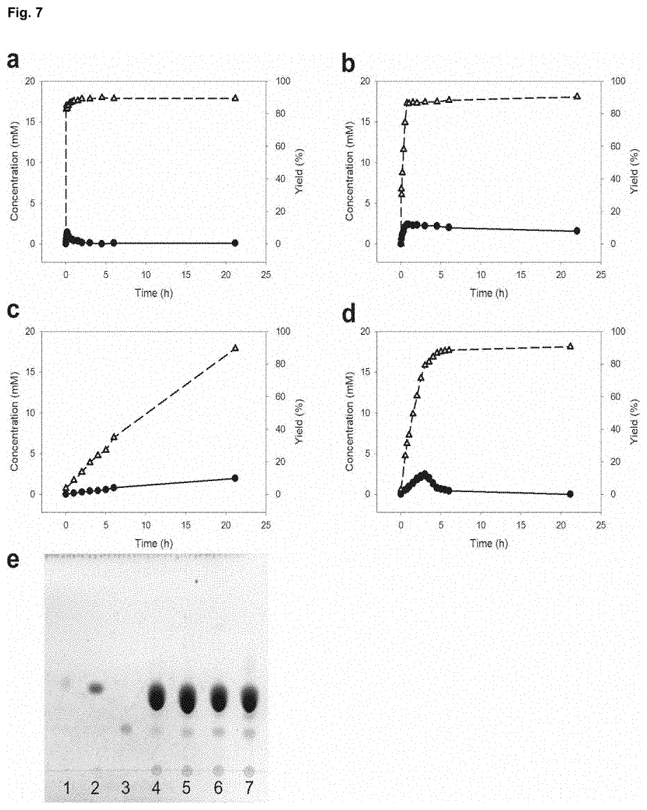

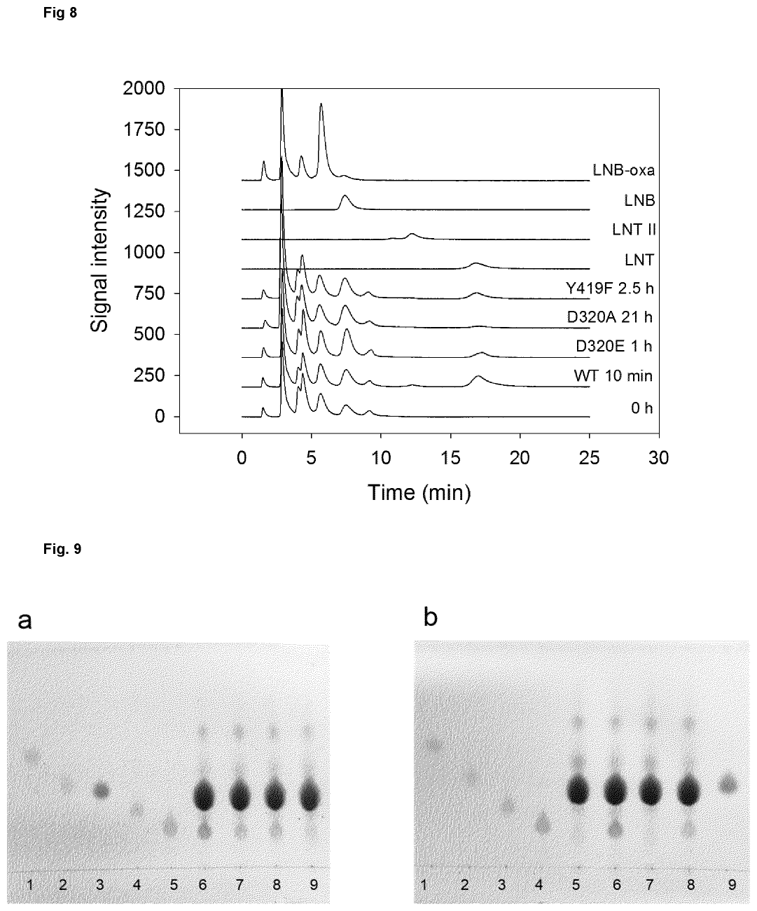

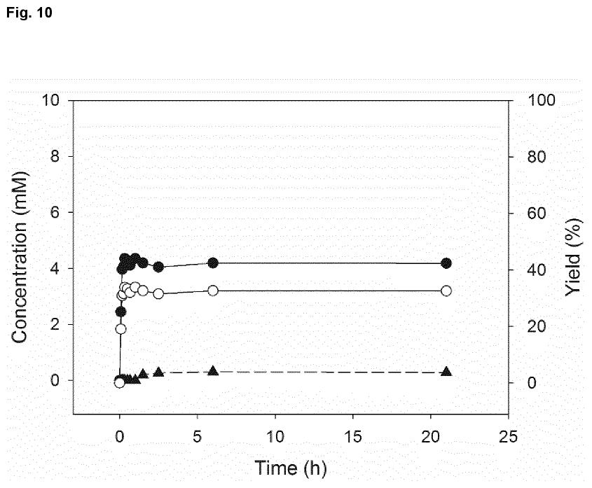

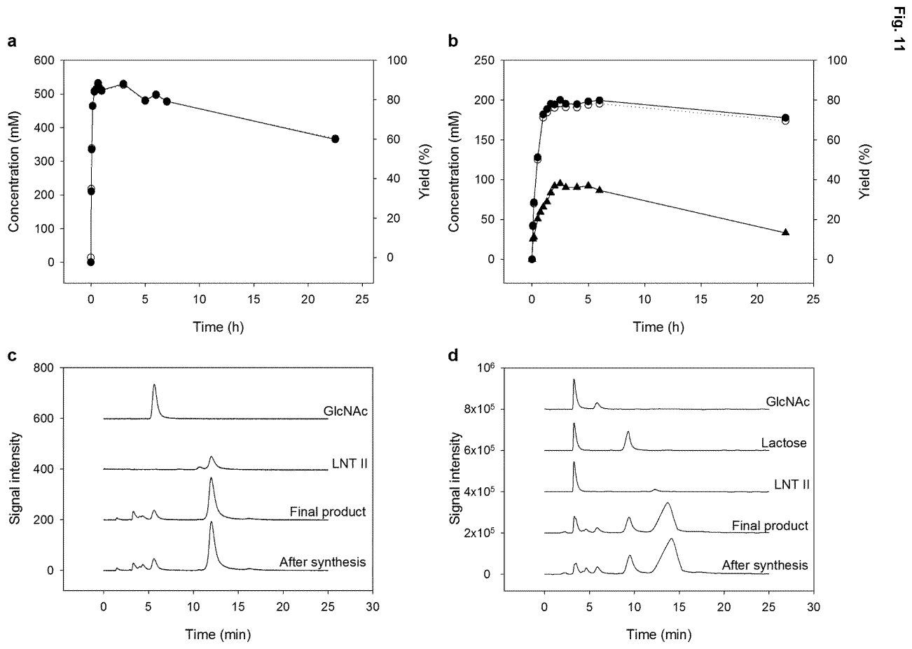

### EXAMPLES

### Materials

Media components and chemicals were of reagent grade from Sigma Aldrich/Fluka (Austria/Germany), Roth (Karlsruhe, Germany) or Merck (Vienna, Austria). HisTrap FF 5 mL column was from GE Healthcare (Vienna, Austria). Minisart® NML syringe membrane filter (0.45 pm) and Vivaspin® Turbo 15 centrifugal concentrators (30 kDa, 50 kDa) were from Sartorius (Goettingen, Germany). GlcNAc, 2-hydroxybenzimidazole (purity 97%), dimethyl sulfone (purity 99.96%) and succinonitrile were from Sigma Aldrich (Austria/Germany). 4-nitrophenyl 2-acet-amido -2-deoxy-β-D-glucopyranoside (GlcNAc-β-pNP), 4-nitrophenyl 2-acetamido-2-deoxy-3-O-(β-D-galactopyranosyl) -β-D-glucopyranoside (LNB-β-pNP), lacto-N-triose II (LNT II), lacto-N-tetraose (LNT, purity ≥90%), lacto-N-biose (LNB) and α-D-galactose-1-phosphate dipotassium salt hydrate (Gal 1-P) were from Carbosynth (Compton, Berkshire, UK). Chromabond Flash FM 70/10C C18 ac adsorbent was von Macherey Nagel (Schoonebeek, Netherlands). Acetonitrile (HPLC gradient grade) was from Chem-Lab NV (Zedelgem, Belgium).

### Example 1: Enzyme Preparation

Production of the enzymes (without signal peptide and transmembrane region/membrane anchor) and their purification were done according to protocols from literatures^{, 6,26} Briefly, synthetic Bbhl genes (wild-type N-acetylhexosaminidase from B. bifidum JCM1254 (GenBank: AB504521.1, aa 33-1599)⁵and D746E, D746A, D746Q, Y827F variants) and synthetic LnbB genes (wild-type lacto-N-biosidase from B. bifidum JCM1254 (GenBank: EU281545.1, aa 35-1064)⁶ and D320E, D320A, Y419F variants) codon-optimized for E. coli expression were ligated into Ndel-Xhol-cut pET21b(+) and pET24b(+) plasmids, respectively (Bio-Cat GmbH, Heidelberg, Germany). Residue numbering of full length enzymes is used. All inserts were confirmed by DNA sequencing. Bbhl enzymes were expressed in E. coli BL21(DE3) at 25° C. for 20 h by induction with 0.5 mM isopropyl-β-D-thiogalactopyranoside (IPTG), using LB-medium supplemented with 115 mg L-1 ampicillin. LnbB enzymes were expressed in E. coli BL21(DE3) following an auto-induction protocol³³ in LB medium with 50 μg mL⁻¹, kanamycin, 25 mM Na₂HPO₄, 25 mM K₂HPO₄, 50 mM NH₁Cl, 5 mM Na₂SO₄, 2 mM MgSO₄, 0.5% glycerol, 0.05% glucose and 0.2% lactose at 110 rpm and 30° C. for 20 h.³⁴ Each enzyme was produced as C-terminal Hiss-tag fusion protein (SEQ ID NOs: 12 to 20) Enzyme purification was done by single-step Hiss-tag affinity chromatography. The enzyme preparations used were (almost) pure by the criterion of migration as single protein band in SDS PAGE.

### Example 2: Protein purification by Hiss-tag Affinity Chromatography

For protein purification, cell pellet from 1 L cell culture was resuspended in 25-30 mL binding buffer (20 mM sodium phosphate, 150 mM NaCl, 15 mM imidazol, pH 7.4) and frozen at −20° C. overnight. 35 mL aliquots of thawed cell suspension were ultrasonicated on ice bath at 60% amplitude for 6 min (2 s pulse on and 4 s pulse off) using a Sonic Dismembrator (Ultrasonic Processor FB-505; Fisher Scientific, Austria) equipped with a 1.27 cm probe for cell disruption. Cell lysates were centrifuged at 4° C. and 21,130 g for 1 h (Eppendorf centrifuge 5424R) and filtered via 0.45 μm cellulose-acetate syringe filters. Target proteins were purified from the cell-free extract via their C-terminal His6-tag using an AktaPrime plus system (GE Healthcare, Germany) at 4° C. The cleared cell lysate was loaded onto a HisTrap FF 5 mL column (GE Healthcare, Austria) at a flow rate of 2 mL min⁻¹. The column had been equilibrated with binding buffer. After a washing step of 15 column volumes (CVs), the enzyme was eluted with 300 mM imidazol within 6 CVs at a flow rate of 4 mL min⁻¹. Target protein containing fractions were pooled. Eluted enzyme was concentrated and buffer exchanged to 20 mM sodium phosphate, 150 mM NaCl, pH 7.4 using Vivaspin® Turbo 15 centrifugal concentrators (30 kDa or 50 kDa, 3645 g, 4° C.). SDS PAGE was used to confirm purity of enzyme preparations. Protein concentrations were measured with a DeNovix SA-11+spectrophotometer (DeNovix Inc, US) at 280 nm. −40 mg of Bbhl and -120 mg of LnbB enzymes, respectively, were typically obtained per liter of culture medium. Purified enzymes were aliquoted and stored at −70° C.

### Example 3: Preparation of 2-chloro-1,3-dimethyl-1H-benzimidazol-3-ium chloride (CDMBI)

CDMBI was prepared as described previously,⁸ with the following modifications. CDMBI was prepared by chemical synthesis in 2 steps. For preparation of 1,3-dimethylbenzim-idazolone (DMBI), the N-atoms of 2-hydroxybenzimidazole were methylated by the action of Mel in the presence of KOH. The DMBI yield could be increased from 70%⁸to 92% by using KOH instead of NaOH, which was previously described in literature.⁸ The reference experiment with NaOH as a base yielded 75% of DMBI.

Step 1: Preparation of 1,3-dimethylbenzimidazolone (DMBI). To a mixture of 50 g 2-hydroxybenzimidazole (1 equiv., 97% purity) and 216.55 g toluene (6.5 equiv.), 5.83 g Bu₄NBr (0.05 equiv.) and 202.86 g KOH (40% w/w, 4 equiv.) were added. The reaction mixture was heated to 60° C. and 118.04 g Mel (2.3 equiv.) were added dropwise within 60 min at high stirring rate. The mixture was stirred for 4 days at 60° C. Note, the reaction time can be reduced to 21 h without any change in yield. After cooling the mixture to 45° C., the phases were separated. The organic layer was washed at 45° C. 3 times with 75 mL 1 N HCl, once with 75 mL saturated NaHCO₃ and dried over Na₂SO₄. After phase separation, the solvent was removed at 50° C. and 150-5 mbar. 58.5 g of crude DMBI were obtained. The residue was recrystallized as follows: crude DMBI was taken up in 90 g acetone/n-heptane (3:1 v/v) at 65° C. The mixture was allowed to cool to room temperature for 16 h, then cooled to 5° C. and stirred for further 3 h. After filtration, the crystalline DMBI was washed once with 40 mL ice-cold n-heptane/acetone (3:1 v/v) and dried under nitrogen to give 53.7 g of DMBI as a white solid (92%) ¹H-NMR (700 MHz, CDCl₃): δ=7.10 (m, 2H), 6.97 (m, 2H), 3.42 (s, 6H). ¹³C-NMR (175 MHz, CDCl₃): δ=27.14, 107.29, 121.17, 129.99, 154.63.

DMBI was converted to CDMBI in a yield of 54% by using oxalyl chloride. When compared to literature, an additional 1.1 equiv. of total oxalyl chloride was used, the reaction temperature was decreased from 80° C. to 70° C., and the reaction times were prolonged.

Step 2: Preparation of 2-chloro-1,3-dimethyl-1H-benzimidazol-3-ium chloride (CDMBI). To a solution of 34.4 g DMBI (1 equiv.) in 271 g toluene (13.9 equiv.), 80 g oxalyl chloride (3 equiv.) were added at 40 ° C. The mixture was heated to 70° C. After 5 d at 70° C. no precipitate was formed. Then, 40 g of oxalyl chloride (1.5 equiv.) were added, and the mixture was stirred at 70° C. overnight. The suspension was cooled to 0-5° C. within 3 h and stirred for further 3 h at this temperature. After filtration, the filter cake was washed with 70 mL of ice-cold toluene and dried in vacuo to give 24.8 g of CDMBI (54%). ¹H-NMR (500 MHz, D₂O): δ=7.85 (m, 2H), 7.72 (m, 2H), 4.08 (s, 3H). ¹³C-NMR (125 MHz, D₂O): δ=35.28, 115.51, 130.08, 134.22, 143.45. In comparison to the protocol reported in literature (49% yield),⁸ an additional 1.1 equiv. of total oxalyl chloride was used, the reaction temperature was decreased from 80° C. to 70° C. and the reaction times were prolonged.

Then, CDMBI and Na₃PO₄were used for oxazoline formation from N-Acetylgucosa-min (GlcNAc) or lacto-N-biose (LNB).⁸

Overall, the CDMBI synthesis was significantly improved. The CDMBI yield over 2 steps could be increased from 34%⁸ to 50% by the modifications just described above.

### Example 4: Preparation of lacto-N-biose (LNB)

LNB was synthesized from Gal 1-P and GlcNAc by lacto-N-biose phosphorylase (LNBP) from Bifidobacterium longum JCM 1217, previously described by Kitaoka and co-work-ers. ^{8,10} LNBP production. Production of the LNBP and purification were done according to protocols from literature.⁹ Briefly, synthetic LNBP gene (GenBank: AB181926.1, aa 20-2275) not codon-optimized for E. coli expression was ligated into Ndel-Xhol-cut pET30a(+) plasmid (GenScript, Piscataway, USA). Insert was confirmed by DNA sequencing. LNBP was expressed in E. coli BL21(DE3) at 30° C. for 20 h by induction with 0.5 mM isopropyl-β-D-thiogalactopyra-noside (IPTG), using LB-medium supplemented with 50 mg L⁻¹ kanamycin. LNBP was produced as C-terminal Hiss-tag fusion protein. Enzyme purification was done by single-step Hiss-tag affinity chromatography (see above). The following buffers were used: binding buffer (20 mM MOPS, 500 mM NaCl, 15 mM imidazol, pH 7.4), elution buffer (20 mM MOPS, 500 mM NaCl, 300 mM imidazol, pH 7.4), storage buffer (20 mM MOPS, 150 mM NaCl, pH 7.5). −50 mg of LNBP was typically obtained per liter of culture medium. The enzyme preparation used was (almost) pure by the criterion of migration as single protein band in SDS PAGE.

Enzymatic synthesis of LNB. Reaction was performed in a total volume of 40 mL using 5.4 mmol Gal 1-P (1.82 g) and 1.8 mmol GlcNAc (0.40 g) dissolved in water. The pH was adjusted to 6.8 with 4 M HCl and the reaction was started by adding 0.05 mg mL⁻¹ (0.6 μM) LNBP. The conversion was performed in a 50 mL Sarstedt tube (diameter 2.8 cm, height 11.5 cm) under magnetic stirring (stir bar: 18×5 mm; 500 rpm) at 37° C. For temperature control, the Sarstedt tube was placed in a water bath. The pH was constantly monitored and manually controlled by adding 4 M HCl (within first 1.5 h). Incubation was for 3.5 h. Samples were taken at certain times and analyzed by HPLC. The reaction yield was 92% (42 mM, 16 g L⁻¹).

Downstream processing (DSP). Major task of the DSP was the removal of Gal 1-P (93 mM) from the LNB (42 mM). Only a small amount of GlcNAc (3 mM) was present. Gal 1-P was removed from the mixture by anion-exchange chromatography (AEC) after enzyme-removal by ultra-filtration (Vivaspin concentrators 30 kDa, 4000 rpm, 20° C.). AEC was performed at pH 7.5. To allow efficient removal of Gal 1-P by binding to the anion-exchange column, the filtrate was 8-fold diluted to an ionic strength of ˜3.6 mS cm⁻¹ with ultra-pure water. LNB and remaining GlcNAc are not ionized at pH 7.5 and elute in the flow-through. AEC was performed on an ÄktaPrime plus system (GE Healthcare, Germany) at room temperature. A self-packed Proteus 20 mL FliQ column (100×16.0 mm, Generon, UK) containing about 15 mL of Toyopearl SuperQ-650M was applied. Ultra-pure water (mobile phase A) and 1 M potassium chloride in ultra-pure water (mobile phase B) were used for binding and elution, respectively. Column was equilibrated with mobile phase A at 4 mL min⁻¹ (5 CVs). 40 mL sample were loaded at a flow rate of 2 mL min⁻¹ using mobile phase A. LNB eluted together with GlcNAc within 5 CVs. Gal 1-P was eluted with mobile phase B at 4 mL min⁻¹ (5 CVs). Detection was by conductivity. Complete removal of Gal 1-P from LNB was verified by TLC analysis. LNB containing fractions were pooled. Sample was concentrated under reduced pressure at 40° C., frozen in liquid nitrogen under rotary motion before freeze-drying overnight (Christ Alpha 1-4, B. Braun Biotech International, Melsungen, Germany). The final product (80% isolated yield) was analyzed by HPLC and its chemical identity confirmed by ¹H NMR. 5% (w/w) of GlcNAc were detected in the final product.

### Example 5: Preparation of Sugar Oxazolines

GlcNAc- and LNB-oxazoline were prepared as described previously.⁸ Briefly, CDMBI (3 equiv.) was used as dehydrative condensing agent and Na3PO4 (7.5 equiv.) as a base for oxazoline formation from GlcNAc (1 equiv.) or LNB (1 equiv.). N-acetyl-2-amino sugars were added and the resulting solution was cooled to 0-3° C. CDMBI (3 equiv.) was added to the solution in portions within 15 min, and the mixture was stirred for 1 h at the same temperature.

GlcNAc is easily available at low cost. Practical preparation of LNB in bulk quantities from sucrose and GlcNAc, using a one-pot four-enzyme reaction with lacto-N-biose phosphory-lase (LNBP) from Bifidobacterium bifidum as a key enzyme, was reported by Kitaoka and co-workers.^{9,10} Analogously, LNB was synthesized from Gal1P and GlcNAc by LNBP. Anion-ex-change chromatography (AEC) and freeze-drying was used for preliminary downstream processing (DSP) (80% isolated yield, purity ≥90%). Initial downstream processing (DSP) of the sugar oxazolines included filtration (Chromabond Flash FM 70/10C C18 ac adsorbent (10 g per g of N-acetyl-2-amino sugar used)) and freeze-drying (lyophilized crude product). Product identities were unequivocally confirmed by ¹H NMR spectroscopy. NMR spectra were in accordance with published data.⁸ The content of sugar oxazolines was determined by quantitative ¹H NMR spectroscopy with dimethyl sulfone or succinonitrile as internal standards. No DMBI was detectable, but the excess of salt remained in the lyophilized crude product. GlcNAc-oxazoline was obtained on 30 mmol scale in a yield of ˜60%. LNB-oxazoline was obtained on 0.5 mmol scale in a yield of 79%.

In order to ensure efficient trans-glycosylation from the sugar oxazolines in the next step, the lyophilized crude products were desalted. Desalting was established by extraction with acetonitrile (9.5 g per g lyophilized crude product, 1 h stirring at room temperature), which is scalable. After filtration, washing (2 * with acetonitrile (3 g per g lyophilized crude product)), concentration and drying in vacuo, GlcNAc-oxazoline was obtained on 3.5 mmol scale in a yield of ˜60%. LNB-oxazoline was obtained on 0.082 mmol scale in a yield of <10%. The yields were determined by HPLC analysis of the N-acetyl-2-amino sugars released (GlcNAc, LNB) after complete hydrolysis of the oxazoline ring.

### Example 6: Bbhl and LnbB Activity Assays

Bbhl and LnbB activities were assayed in a total volume of 600 μL and 400 μL, respectively, using 50 mM sodium phosphate buffer, pH 7.5 (with oxazoline donor substrates) or pH 5.8 (with pNP-labelled donor substrates). Reaction mixture with sugar oxazoline donor substrate contained 60 mM GlcNAc-oxazoline or 12 mM LNB-oxazoline, 600 mM lactose and 0.23-18 μM enzyme. Reaction mixture with pNP-labelled donor substrate contained 20 mM GlcNAc-β-pNP or LNB-β-pNP, 15-20% DMSO, 400-600 mM lactose and 0.12-24 μM enzyme. Enzymatic conversion was carried out at 37° C. or 55° C. (as indicated in the text; see Table 1) and agitation rate of 650 rpm using a Thermomixer comfort (Eppendorf, Germany).

Reactions were stopped at certain times by heating for 10 min at 99° C. Precipitated protein was removed by centrifugation at 13,200 rpm for 10 min. Samples were analyzed by hydrophilic interaction liquid chromatography (HILIC-HPLC) and thin layer chromatography (TLC).

Specific activities were calculated from initial rates of product formation (trans-glyco-sylation) and product hydrolysis (secondary hydrolysis), respectively, obtained at 400-600 mM of lactose (synthesis conditions'). One unit (1 U) of trans-glycosylation activity was defined as the amount of enzyme that could transfer 1 μmol of N-acetyl-2-amino sugar (Bbhl: GlcNAc; LnbB: LNB) per min to lactose under the conditions described above. One unit (1 U) of secondary hydrolysis activity was defined as the amount of enzyme that could release 1 μmol of N-acetyl -2-amino sugar (Bbhl: GlcNAc; LnbB: LNB) per min from the product formed under the conditions described above. For R_{TH} analysis, specific activities for total (productive and non-pro-ductive) GlcNAc-β-pNP or LNB-β-pNP hydrolysis (based on pNP released) and trans-glycosylation were calculated from initial rate data obtained at 400-600 of lactose. The difference gave the specific activity for non-productive donor substrate hydrolysis (primary hydrolysis). For the oxazoline substrates, the total donor hydrolysis could only be calculated based on initial rate data of release of N-acetyl-2-amino sugar (Bbhl: GlcNAc; LnbB: LNB) for the variants of Bbhl and LnbB having their catalytic Asp replaced by Glu. In all other cases, the R_{TH} values were estimated based on endpoint measurements at maximum LNT II or LNT yield.

### Example 7: Preparative-Scale Synthesis of LNT II

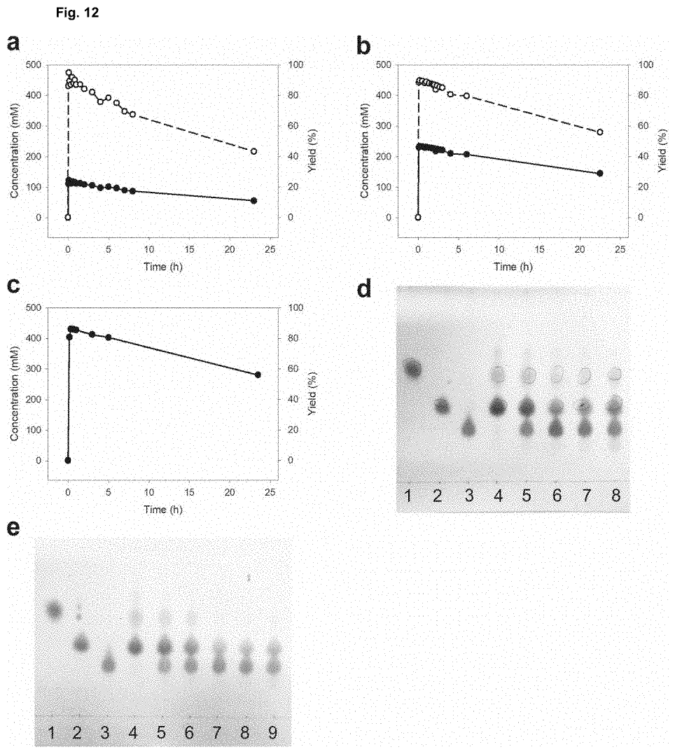

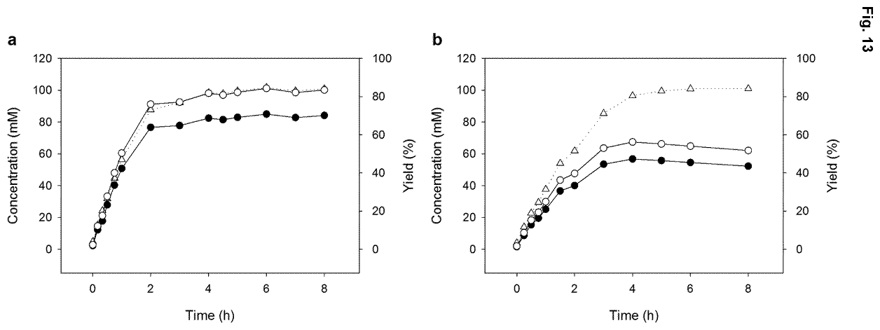

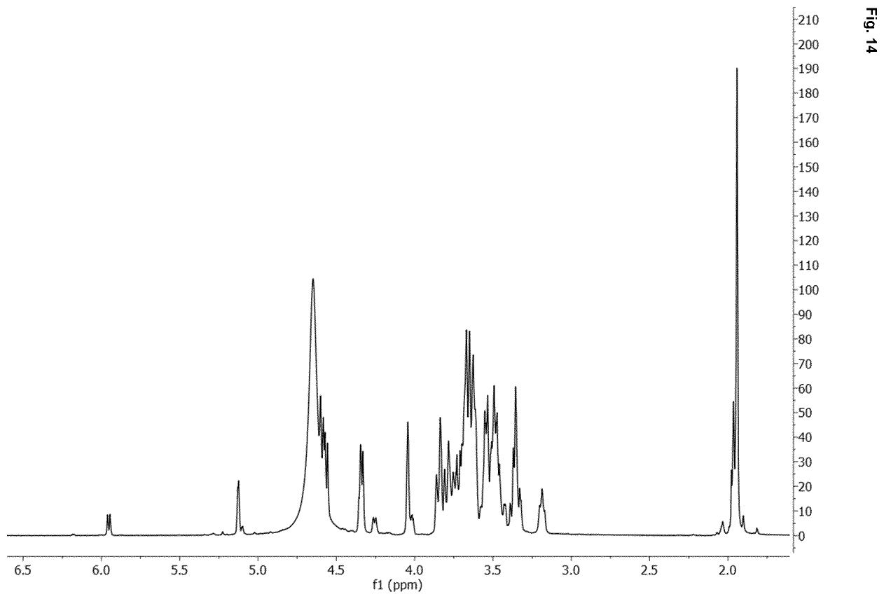

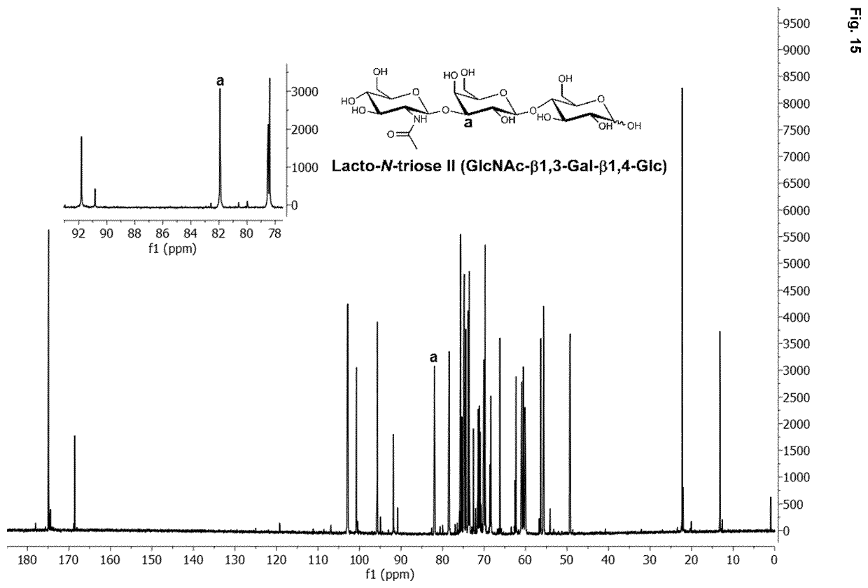

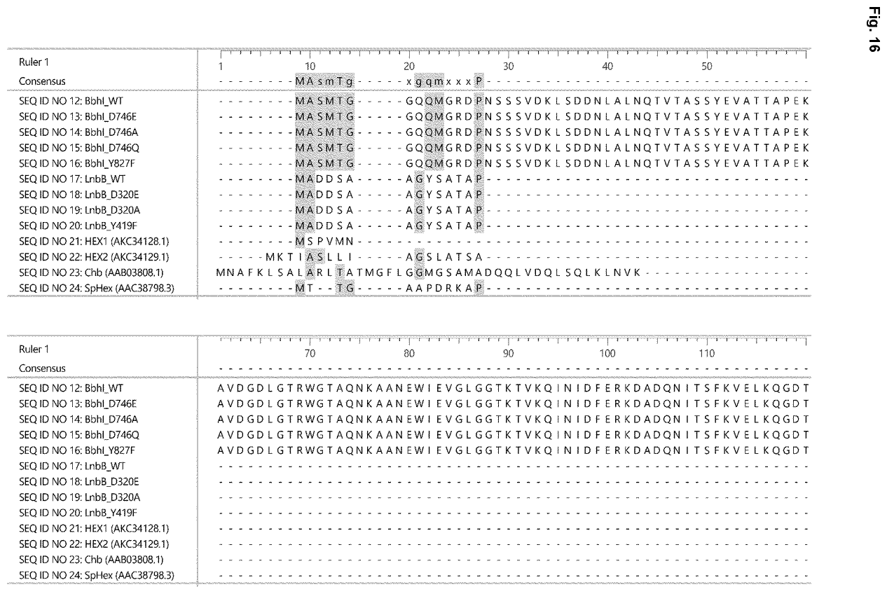

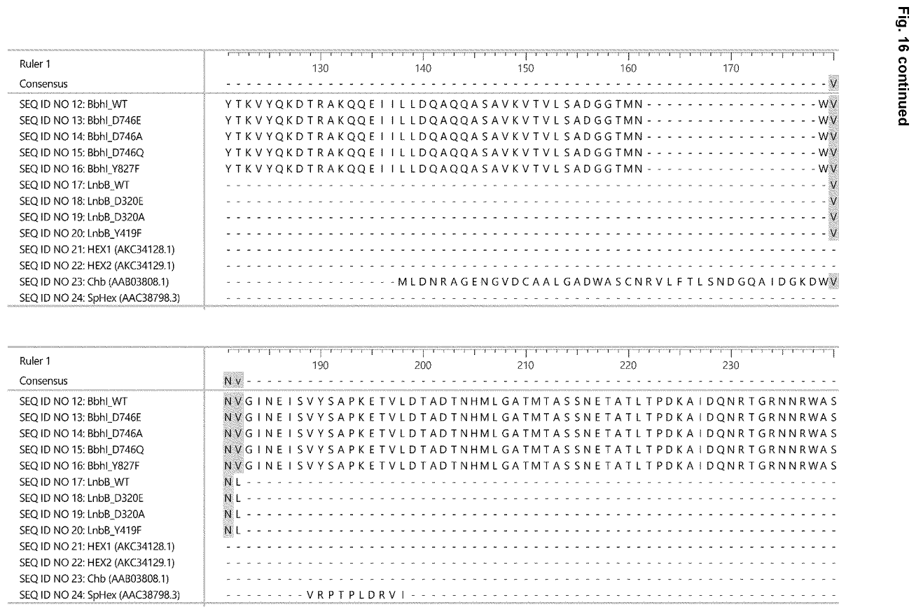

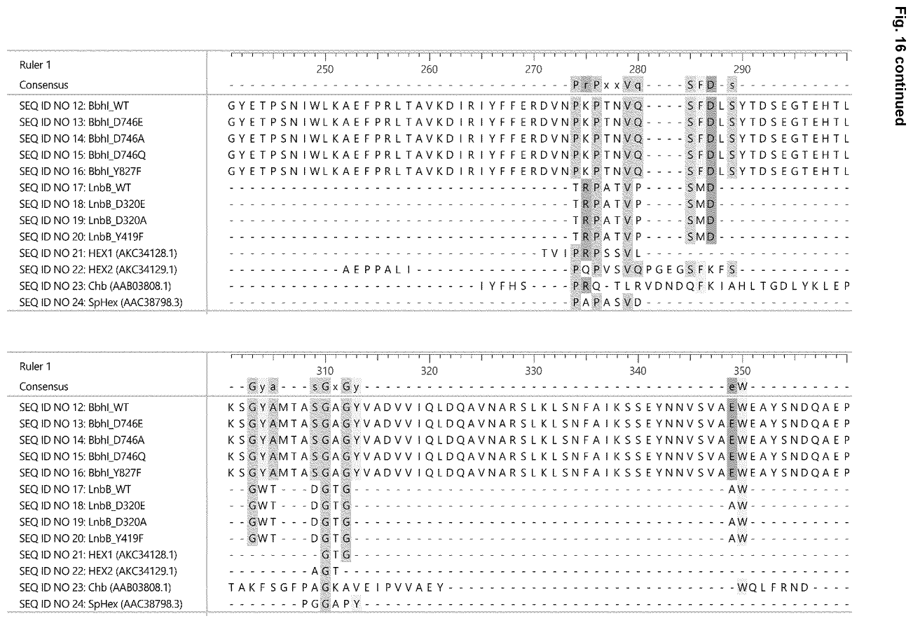

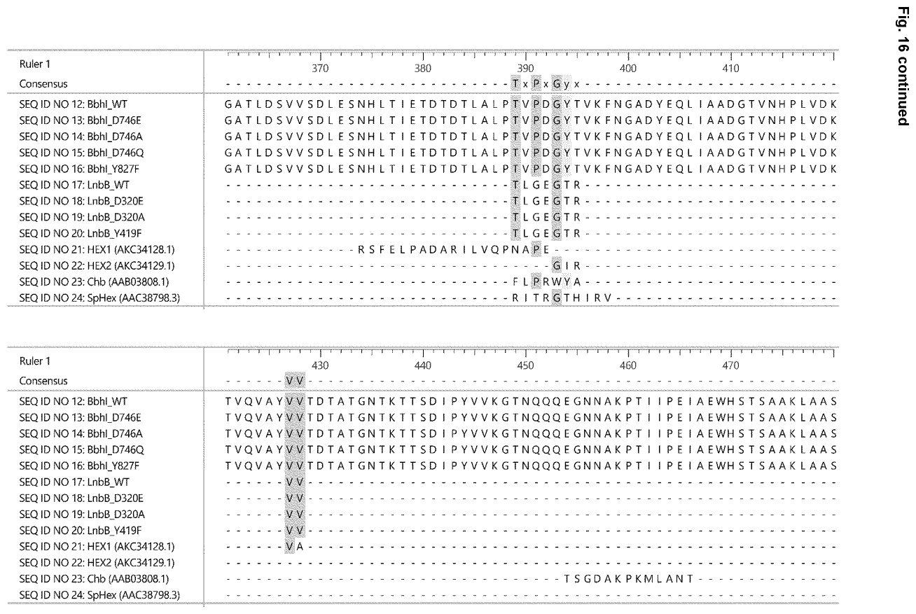

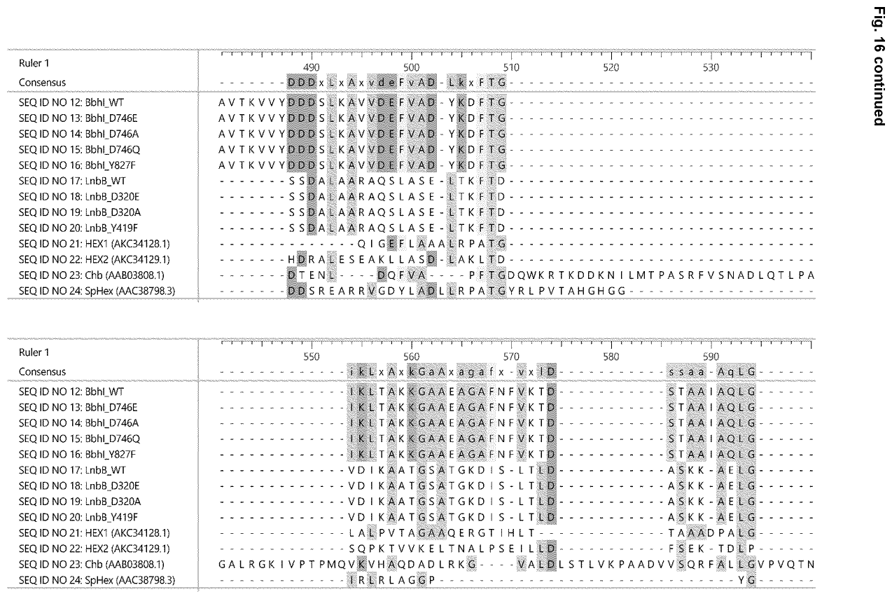

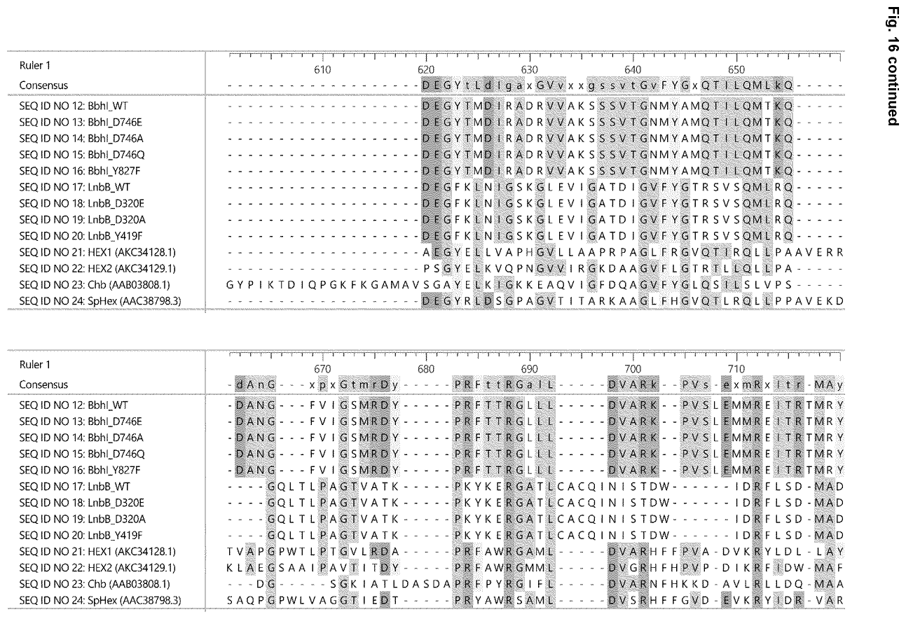

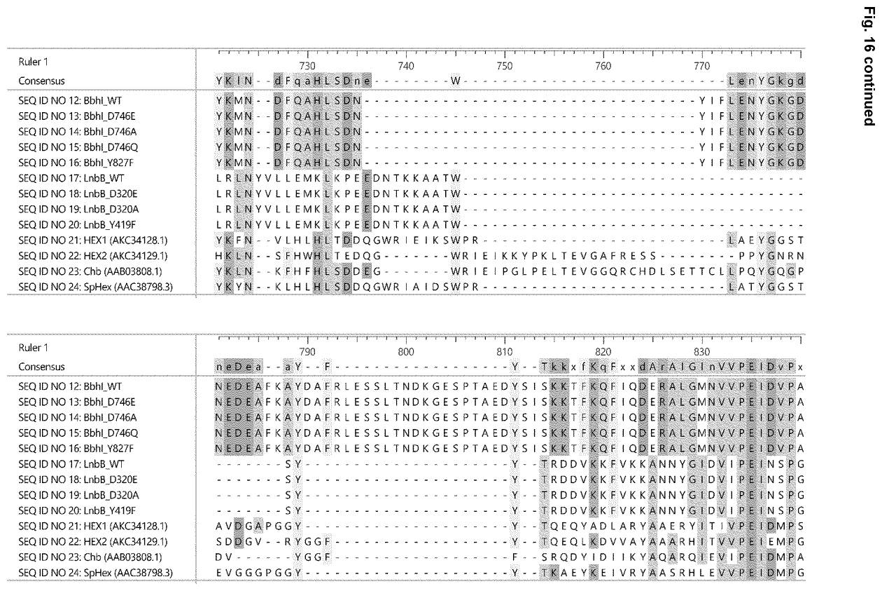

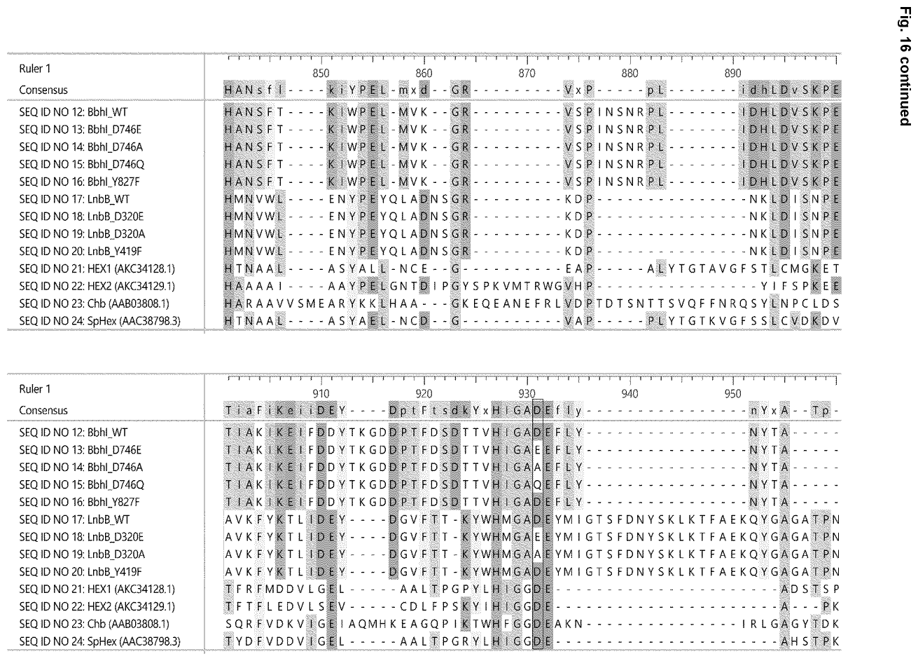

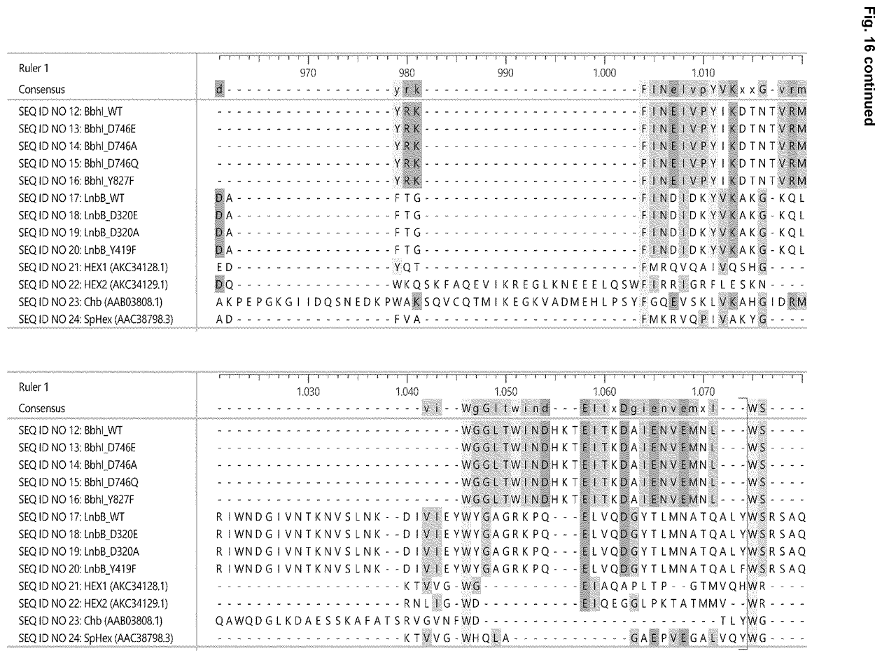

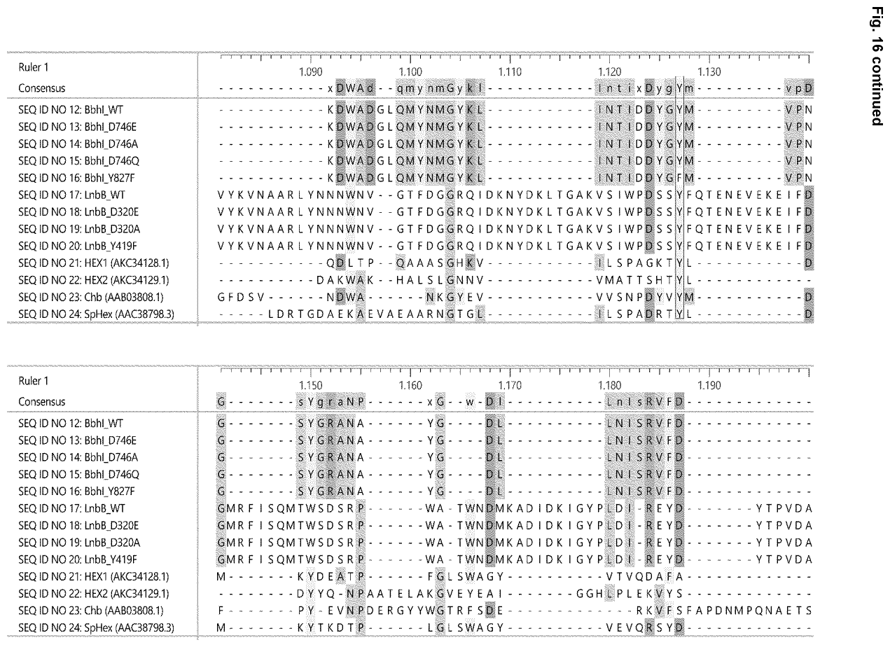

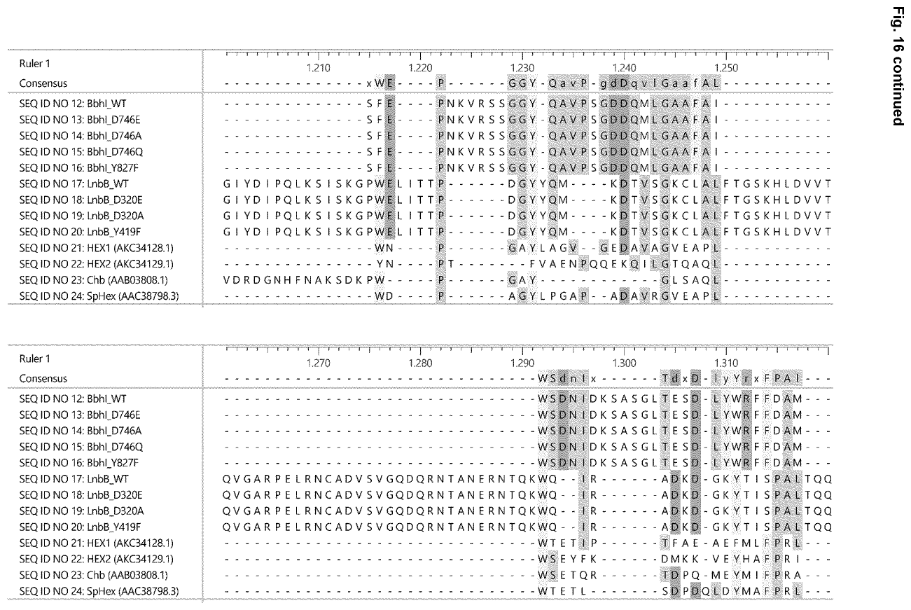

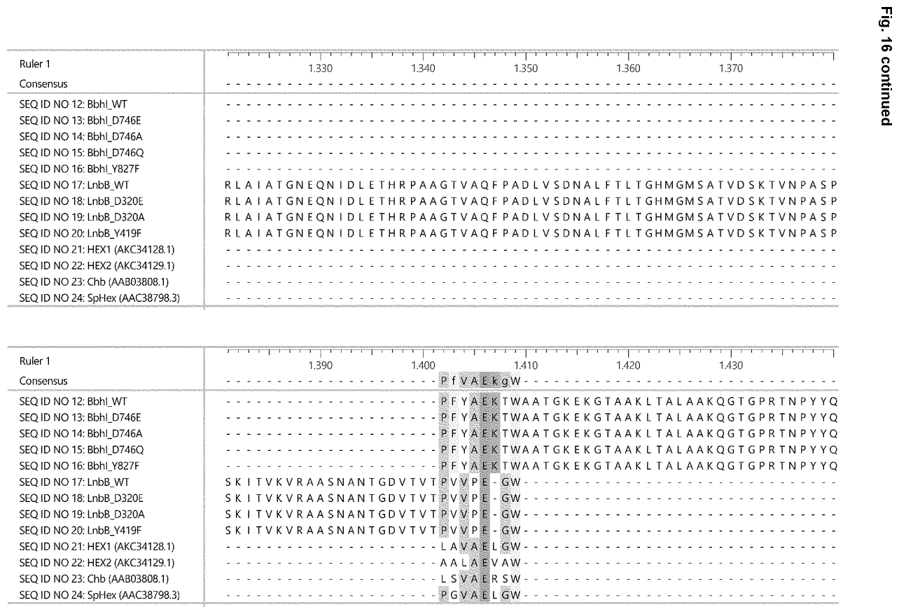

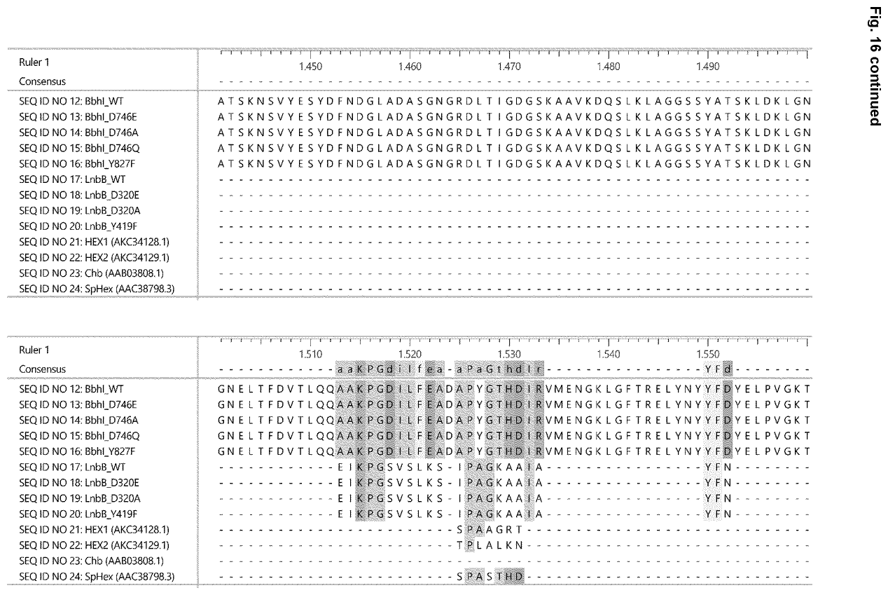

In order to allow bulk synthesis of LNT II, its synthesis from GlcNAc-oxazoline by the D746E glycosidase mutant of Bbhl had to be optimized with respect to the donor-to-acceptor ratio applied. Increasing concentrations of GlcNac-oxazoline (130-600 mM) were applied at a constant lactose concentration of 600 mM and the conversions compared at 37° C. (FIG. 11a, FIG. 12). The adjustment of the donor-to-acceptor ratio to 1 had no negative impact on the final yield (85-90%). FIG. 11a shows synthesis of ˜1 g of LNT II in a batch volume of only 3.6 mL under the optimized conditions. The initial LNT II production rate was 2190 g L⁻¹ h⁻¹. LNT II was obtained in excellent yield (85%) and concentration (281 g L⁻¹, 515 mM) within 30 min of reaction. The STY of the biotransformation overall was 562 g L⁻¹ h⁻¹. The mass-based turnover number (g product formed per g enzyme added; TTNmass) reached a value of 388. Only marginal product hydrolysis was observed under the reaction conditions applied. The ratio of transglycosylation over secondary hydrolysis reached a value of ˜1800. When the D746E variant and the wild-type enzyme were assayed under exactly the same conditions (using one-tenth of the enzyme concentration compared to the bulk synthesis described above), one sees clearly the benefits of the new glycosidase, namely no secondary hydrolysis and doubling of the LNT II yield (FIG. 11b). The initial LNT II production rates of the two enzymes were comparable, but the conversion with the wild-type drastically slowed down already after 5 min while it remained constant over ˜0.5 h with the mutant. Note, 600 mM of each substrate was the upper concentration limit used in the reaction, allowing their full solubility. LNT II was obtained in 80% yield without any detectable hydrolysis of the product (FIG. 13a). Under these conditions, the wild-type yielded ˜50% of LNT II and showed also an improved trans-glycosylation to secondary hydrolysis ratio of ˜40 (FIG. 13b).

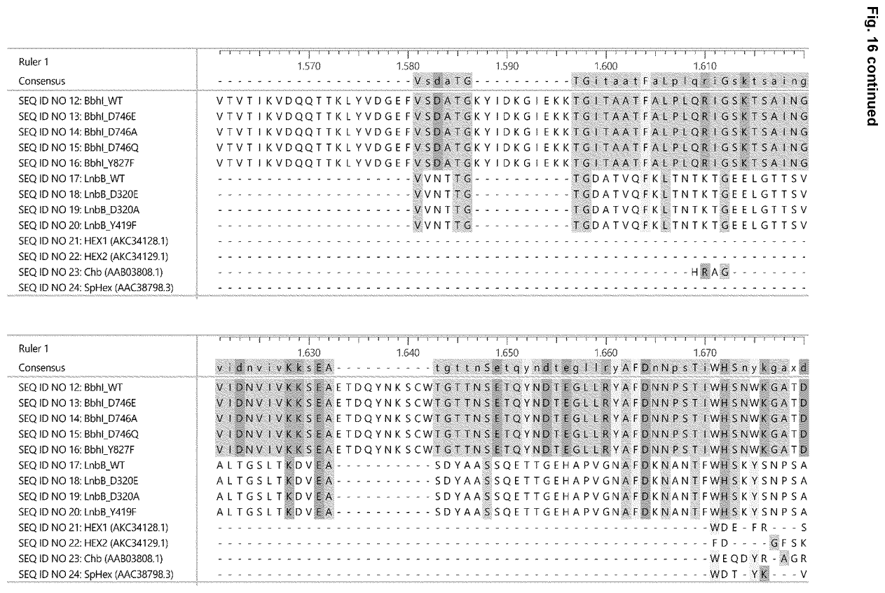

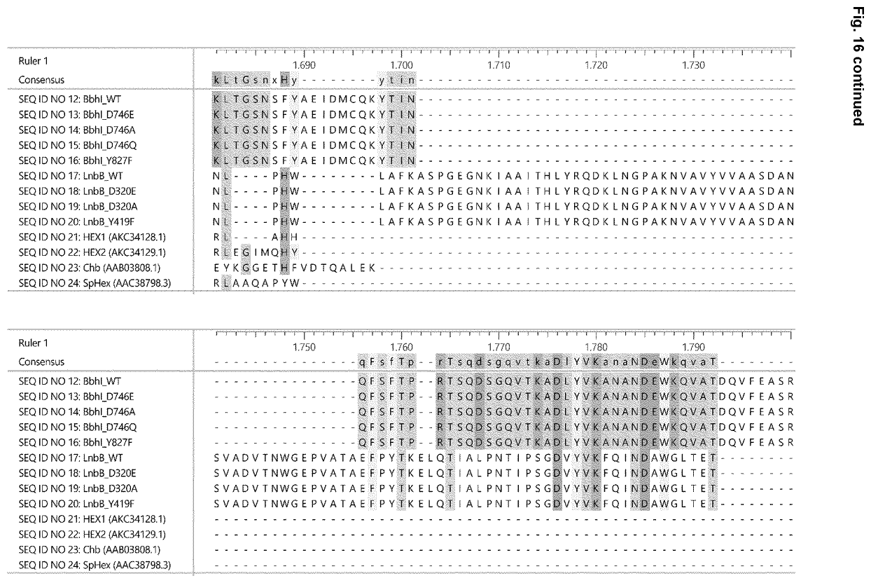

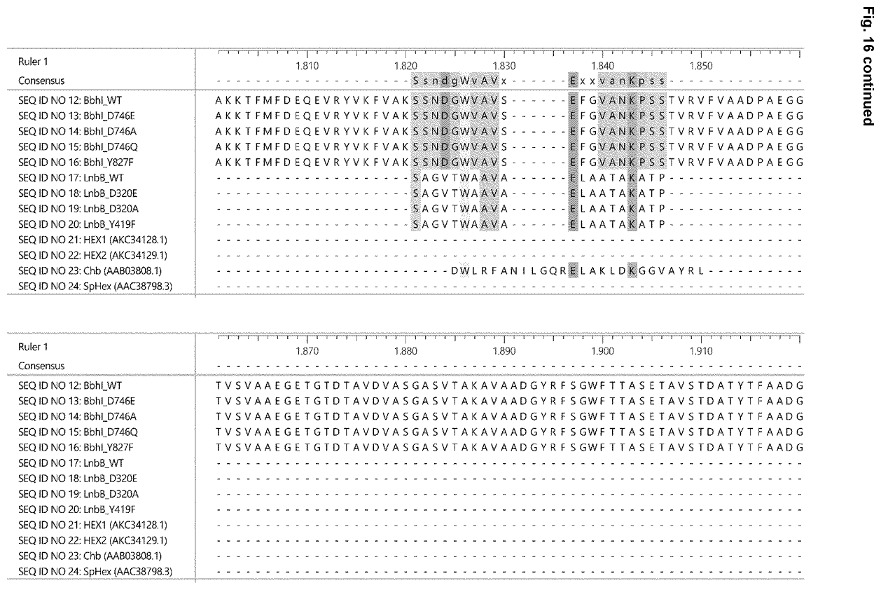

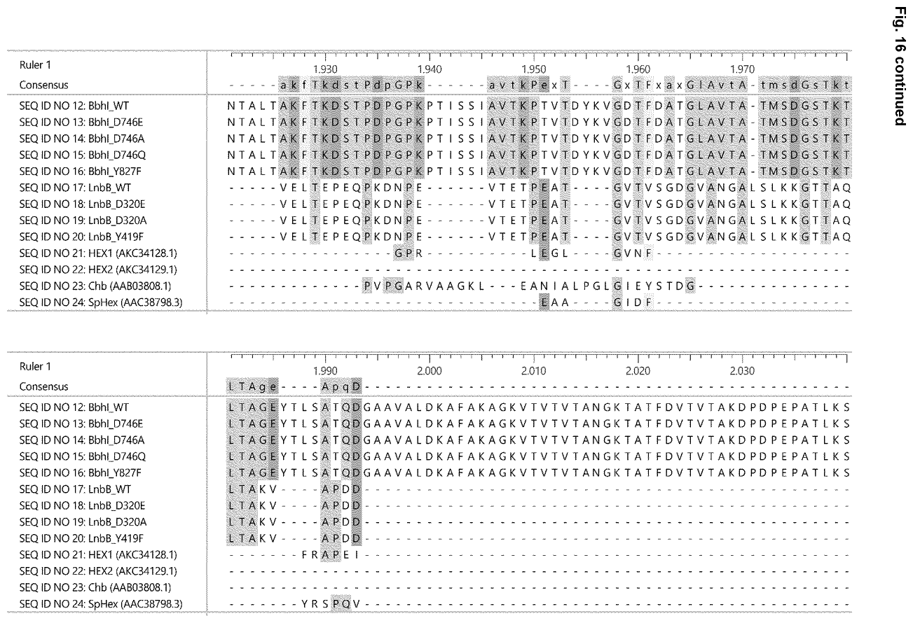

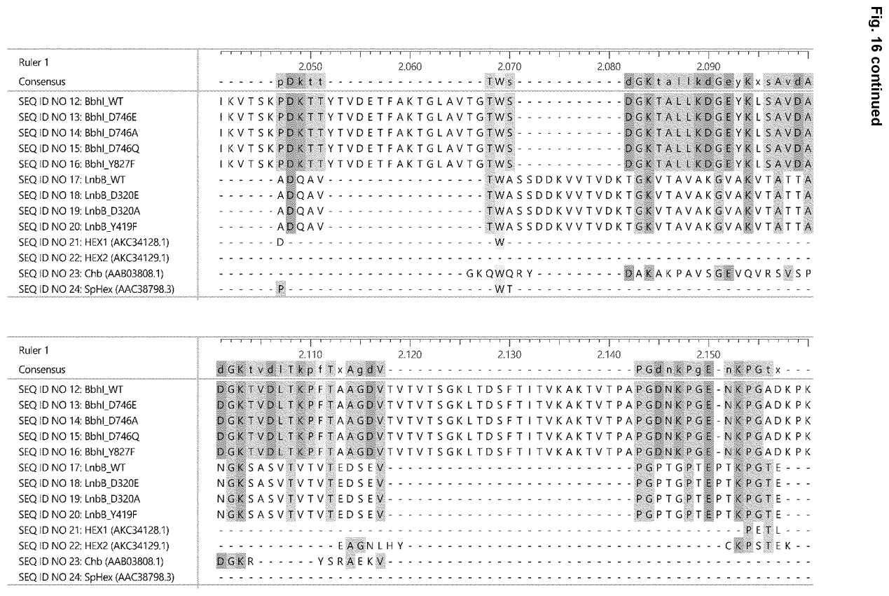

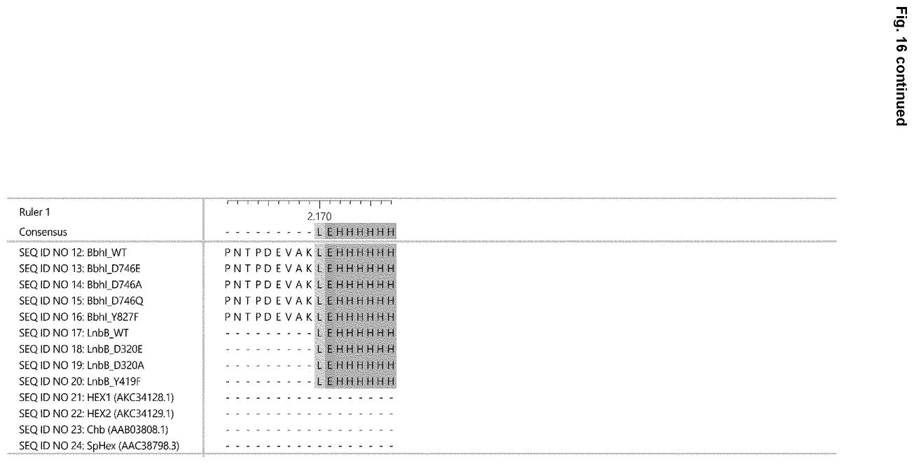

For DSP of LNT II, the reaction was stopped by heating when no further increase in product concentration was detected. The sample contained only 85 mM of GlcNAc and lactose next to 515 mM of LNT II. Therefore, a simple DSP, including centrifugation for enzyme removal and freeze-drying for water removal, was sufficient to isolate LNT II in a purity of ˜80% (based on LNT II content of the final product, FIGS. 11c,d). The main residual impurities were 5% (w/w) GlcNAc and 10% (w/w) lactose. If a higher product purity is required, nanofiltration could be used for removal of GlcNAc and lactose from the LNT ³⁶ About 1 g of LNT II was obtained as a white powder in ≥85% yield. LNT II was thus prepared from GlcNAc-oxazoline in 73% overall yield. Product identity was unequivocally confirmed by ¹H and ¹³C NMR spectroscopy (FIGS. 14 and 15). NMR spectra were in accordance with published data.⁵ LNT II was the only regioisomer detected (FIG. 15). Overlay of NMR spectra (¹³C, HSQC) of isolated LNT II (FIG. 15) and commercial standard showed exact match of the signal at 82 ppm, characteristic for β-GIcNAc linked to the C-3 position of the β-galactosyl residue of lactose.

The enzymatic conversion was carried out at pH 7.5 and 37° C. in a total volume of 3.6 mL, using equimolar amounts of GlcNAc-oxazoline and lactose (600 mM). Reaction was started by adding 0.73 mg mL⁻¹ (4 μM) of Bbhl D746E variant. The conversion was performed in a 50 mL Sarstedt tube (diameter 2.8 cm, height 11.5 cm) under magnetic stirring (stir bar: 18×5 mm; 250 rpm). For temperature control, the Sarstedt tube was placed in a water bath. Samples were taken at certain times and analyzed by HPLC.

For downstream processing (DSP) of LNT II, enzyme was precipitated after 45 min reaction time by heating for 15 min at 99° C. Precipitated protein was removed by centrifugation at 13,200 rpm for 15 min. Sample was frozen at −70° C. and freeze-dried overnight (Christ Alpha 1-4, B. Braun Biotech International, Melsungen, Germany). The final product was analyzed by HPLC and its chemical identity confirmed by NMR.

### Example 8: Synthesis of LNT II by Fed-Batch Addition of GIcNAc-oxazoline

1.8 mmol GlcNAc-oxazoline was dissolved in 1.3 mL ice-cold water and added continuously over a period of 65 min to the reaction solution containing 2.1 mmol of lactose and 0.3 mg (0.4 βM) of Bbhl D746E in 2.3 mL of phosphate buffer pH 7.5. The enzymatic conversion was carried out at 37° C. under magnetic stirring. For temperature control, the reaction tube was placed in a water bath. Samples were taken at certain times and analyzed by HPLC. Formation of the product was demonstrated. The reaction was terminated after 4 h by heat deactivation of the enzyme.

### Analytics

LNT II, LNT, GlcNAc, LNB, pNP and lactose were analyzed by HILIC-HPLC using a Luna® NH₂ column (3 μm, 100 Å, 250×4.6 mm; Phenomenex, Germany). HPLC analysis was performed at 30° C. with a mobile phase of 75% acetonitrile and 25% water at an isocratic flow rate of 1 mL min¹. UV-detection at 195 nm was used for quantification of LNT II, LNT, GlcNAc, LNB and pNP. For preparative synthesis of LNT II, lactose was monitored by refractive index (Rl) detection.

TLC was performed on silica gel 60 F254 aluminum sheet (Merck, Germany). The plate was developed in a solvent system of 1-butanol—acetic acid—water (2/1/1 by volume). TLC plates were analyzed under UV light (254 nm). Then carbohydrates were visualized by heating the plate after spraying it with thymol—sulfuric acid reagent.

LNT II, LNT, GlcNAc, LNB, pNP and lactose were used as authentic standards.

Varian (Agilent) INOVA 500-MHz NMR spectrometer (Agilent Technologies, Santa Clara, Calif., USA) and the VNMRJ 2.2D software were used for all NMR measurements. Dimethyl sulfone and succinonitrile were used as internal standards for quantitative ¹H NMR measurements. 19.08 mg of GlcNAc-oxazoline (lyophilized crude product) and 11.28 mg dimethyl sulfone were dissolved in D2O. 11.41 mg of LNB-oxazoline (lyophilized crude product) and 12.85 mg succinonitrile were dissolved in D₂O. ˜200 mg of isolated LNT II were dissolved in 600 μL D20. Commercial LNT II standard (from Carbosynth; 65 mM) was dissolved in D₂O -H₂O (11.5:1 v/v). ¹H NMR spectra (499.98 MHz) were measured on a 5 mm indirect detection PFG-probe, while a 5 mm dual direct detection probe with z-gradients was used for ¹³C NMR spectra (125.71 MHz). Standard pre-saturation sequence was used: relaxation delay 2 s; 90° proton pulse; 2.048 s acquisition time; spectral width 8 kHz; number of points 32 k. ¹³C NMR spectra were recorded with the following pulse sequence: standard ¹³C pulse sequence with 45° carbon pulse, relaxation delay 2 s, Waltz decoupling during acquisition, 2 s acquisition time. The HSQC spectrum was measured with 128 scans per increment and adiabatic carbon 180° pulses. Mnova 9.0 was used for evaluation of spectra.

### REFERENCES

1. Fujita, M., Shoda, Haneda, K., Inazu, T., Takegawa, K., Yamamoto, K. A novel disac-charide substrate having 1,2-oxazoline moiety for detection of transglycosylating activity of endoglycosidases. Biochim. Biophys. Acta Gen. Subj. 1528, 9-14 (2001).

2. Kobayashi, A., Kuwata, H., Kohri, M., Izumi, R., Watanabe, T., Shoda, S. i. A bacterial chitinase acts as catalyst for synthesis of the N-linked oligosaccharide core trisaccharide by employing a sugar oxazoline substrate. J. Carbohyd. Chem. 25, 533-541 (2006).

3. Wang, L.-X., Huang, W. Enzymatic transglycosylation for glycoconjugate synthesis. Curr. Opin. Chem. Biol. 13, 592-600 (2009).

4. Ochiai, H., Huang, W., Wang, L.-X. Endo-p-N-acetylglucosaminidase-catalyzed polymerization of β-Glcp-(1→4)-GlcpNAc oxazoline: a revisit to enzymatic transglycosylation. Carbohydr. Res. 344, 592-598 (2009).

5. Chen, X., et al. Efficient and regioselective synthesis of β-GaINAc/GIcNAc-lactose by a bi-functional transglycosylating β-N-acetylhexosaminidase from Bifidobacterium bifidum. Appl. Environ. Microbiol. 82, 5642-5652 (2016).

6. Wada, J., et al. Bifidobacterium bifidum lacto-N-biosidase, a critical enzyme for the degradation of human milk oligosaccharides with a type 1 structure. Appl. Environ. Microbiol. 74, 3996-4004 (2008).

7. Jamek, S. B., et al. Loop protein engineering for improved transglycosylation activity of a β-N-acetylhexosaminidase. ChemBioChem 19, 1858-1865 (2018).

8. Noguchi, M., Fujieda, T., Huang, W. C., Ishihara, M., Kobayashi, A., Shoda, S.-i. A practical one-step synthesis of 1,2-oxazoline derivatives from unprotected sugars and its application to chemoenzymatic β-N-acetylglucosaminidation of disialo-oligosaccharide. Helv. Chim. Acta 95, 1928-1936 (2012).

9. Kitaoka, M. & Nishimoto, M. Method for producing lacto-N-biose I and galacto-N-biose. U.S. Pat. No. 8,173,399B2 (2012).

10. Nishimoto, M., Kitaoka, M. Practical preparation of lacto-N-biose I, a candidate for the bifidus factor in human milk. Biosci. Biotechnol. Biochem. 71, 2101-2104 (2007).

11. Slamova, K., et al. Synthesis of derivatized chitooligomers using transglycosidases engineered from the fungal GH2O β-N-acetylhexosaminidase. Adv. Synth. Catal. 357, 1941-1950 (2015).

12. Miwa, M., et al. Cooperation of β-galactosidase and β-N-acetylhexosaminidase from bifidobacteria in assimilation of human milk oligosaccharides with type 2 structure. Glycobiology 20, 1402-1409 (2010).

13. André-Miral, C., et al. De novo design of a trans-β-N-acetylglucosaminidase activity from a

GH1 8-glycosidase by mechanism engineering. Glycobiology 25, 394-402 (2015).

14. Martinez, E. A., et al. Engineering chitinases for the synthesis of chitin oligosaccharides: catalytic amino acid mutations convert the GH-18 family glycoside hydrolases into transgly-cosylases. J. Mol. Catal. B: Enzym. 74, 89-96 (2012).

15. Shoda, S.-i., et al. Efficient method for the elongation of the N-acetylglucosamine unit by combined use of chitinase and β-galactosidase. Helv. Chim. Acta 85, 3919-3936 (2002).

16. Murata, T., Inukai, T., Suzuki, M., Yamagishi, M., Usui, T. Facile enzymatic conversion of lactose into lacto-N-tetraose and lacto-N-neotetraose. Glycoconj. J. 16, 189-195 (1999).

17. Baumgärtner, F., Sprenger, G. A., Albermann, C. Galactose-limited fed-batch cultivation of Escherichia coli for the production of lacto-N-tetraose. Enzyme Microb. Technol. 75-76, 37-43 (2015).

18. Blixt, O., van Die, I., Norberg, T., van den Eijnden, D. H. High-level expression of the Neisseria meningitidis IgtA gene in Escherichia coli and characterization of the encoded N-acetylglucosaminyltransferase as a useful catalyst in the synthesis of GIcNAcβ1→3Gal and GaINAcβ1→3Gal linkages. Glycobiology 9, 1061-1071 (1999).

19. Chen, C., et al. Sequential one-pot multienzyme (OPME) synthesis of lacto-N-neotetraose and its sialyl and fucosyl derivatives. Chem. Commun. 51, 7689-7692 (2015).

20. Johnson, K. F. Synthesis of oligosaccharides by bacterial enzymes. Glycoconj. J. 16, 141-146 (1999).

21. Yu, H., et al. Synthetic disialyl hexasaccharides protect neonatal rats from necrotizing enterocolitis. Angew. Chem. Int. Edit. 53, 6687-6691 (2014).

22. Priem, B., Gilbert, M., Wakarchuk, W. W., Heyraud, A., Samain, E. A new fermentation process allows large-scale production of human milk oligosaccharides by metabolically engineered bacteria. Glycobiology 12, 235-240 (2002).

23. Prieto, P. A., Kleman-Leyer, K. M. Process for synthesizing oligosaccharides. U.S. Pat. No. 5,945,314A (1999).

24. Baumgartner, F., Conrad, J., Sprenger, G. A., Albermann, C. Synthesis of the human milk oligosaccharide lacto-N-tetraose in metabolically engineered, plasmid-free E. coli. Chem-BioChem 15, 1896-1900 (2014).

25. Kobayashi, S., Kiyosada, T., Shoda, S.-i. A novel method for synthesis of chitobiose via enzymatic glycosylation using a sugar oxazoline as glycosyl donor. Tetrahedron Lett. 38, 2111-2112 (1997).

26. Ochiai, H., Ohmae, M., Kobayashi, S. Enzymatic glycosidation of sugar oxazolines having a carbon/late group catalyzed by chitinase. Carbohydr. Res. 339, 2769-2788 (2004).

27. Kohri, M., Kobayashi, A., Shoda, S.-i. Design and utilization of chitinases with low hydrolytic activities. Trends Glycosci. Glycotechnol. 19, 165-180 (2007).

28. Hattie, M., et al. Gaining insight into the catalysis by GH20 lacto-N-biosidase using small molecule inhibitors and structural analysis. Chem. Commun. 51, 15008-15011 (2015).

29. Ito, T., et al. Crystal structures of a glycoside hydrolase family 20 lacto-N-biosidase from Bifidobacterium bifidum. J. Biol. Chem. 288, 11795-11806 (2013).

30. Liu, X.-w., et al. Characterization and synthetic application of a novel β1,3-galactosyltransferase from Escherichia coli O55:H7. Bioorg. Med. Chem. 17, 4910-4915 (2009).

31. Zeuner, B., Nyffenegger, C., Mikkelsen, J. D., Meyer, A. S. Thermostable β-galactosidases for the synthesis of human milk oligosaccharides. New Biotechnol. 33, 355-360 (2016).

32. Bode, L. Human milk oligosaccharides: Every baby needs a sugar mama. Glycobiology 22, 1147-1162 (2012).

33. Studier, F. W. Protein production by auto-induction in high-density shaking cultures. Protein Expr. Purif. 41, 207-234 (2005).

34. Val-Cid, C., Biarnés, X., Faijes, M., Planas, A. Structural-functional analysis reveals a specific domain organization in family GH20 hexosaminidases. PLOS ONE 10, e0128075 (2015).

35. Goulas, A. K., Kapasakalidis, P. G., Sinclair, H. R., Rastall, R. A., Grandison, A. S. Purification of oligosaccharides by nanofiltration. J. Membrane Sci. 209, 321-335 (2002).

36. Nordvang, R. T., et al. Separation of 3′-sialyllactose and lactose by nanofiltration: a tradeoff between charge repulsion and pore swelling induced by high pH. Sep. Purif. Technol. 138, 77-83 (2014).

1. A method for producing lacto-N-triose II and/or lacto-N-tetraose comprising, the step of:
(i) reacting glucosamine-oxazoline and/or lacto-N-biose-oxazoline with lactose catalysed by an enzyme of the glycoside hydrolase family 20 (GH20) to obtain lacto-N-triose II and/or lacto-N-tetraose.

2. Method according to claim 1, wherein step (i) is performed in an aqueous solution containing at least 50 g/L lactose, preferably at least 100 g/L lactose, particularly preferably at least 150 g/L lactose, most preferably at least 190 g/L lactose.

3. Method according to claim 1, wherein the method is performed under conditions that are free or essentially free of organic solvents.

4. Method according to claim 1, wherein the glucosamine-oxazoline and/or lacto-N-biose-oxazoline, respectively, is added to the lactose and the enzyme of the glycoside hydrolase family 20 (GH20) over a period of at least 20 minutes, preferably at least 60 minutes.

5. The method for producing lacto-N-triose II according to claim 1, wherein the enzyme of the glycoside hydrolase family 20 (GH20) is a β-N-acetylhexosaminidase, preferably β-N-acetylhexosaminidase of Bifidobacterium bifidum JCM1254 having an amino acid sequence of SEQ ID NO: 1 or an enzyme having an amino acid sequence identity of at least 70% to SEQ ID NO: 1 and having β-N-acetylhexosaminidase activity, or a β-N-acetylhexosaminidase enzyme comprising an amino acid sequence of SEQ ID NO: 2, or a β-N-acetylhexosaminidase enzyme comprising an amino acid sequence having an amino acid sequence identity of at least 70% to SEQ ID NO: 2.

6. Method for producing lacto-N-tetraose according to claim 1, wherein the enzyme of the glycoside hydrolase family 20 (GH20) is a lacto-N-biosidase, preferably lacto-N-biosidase of Bifidobacterium bifidum JCM1254 having an amino acid sequence of SEQ ID NO: 3 or an enzyme having an amino acid sequence identity of at least 70% to SEQ ID NO: 3 and having lacto-N-biosidase activity, or a lacto-N-biosidase enzyme comprising an amino acid sequence of SEQ ID NO: 4, or a lacto-N-biosidase enzyme comprising an amino acid sequence having an amino acid sequence identity of at least 70% to SEQ ID NO: 4.

7. Method according to claim 1, wherein the method further comprises the step of:
(ii) deactivating the enzyme of the glycoside hydrolase family 20 (GH20) after the reacting step.

8. Method for producing lacto-N-triose II according to any-of-elaims claim 1 further comprising the step:
(iii) adding a β-galactosidase or a galactosyl transferase and UDP-galactose, to the mixture of step (i) or, preferably to the mixture of step (ii), and optionally adding further lactose to obtain lacto-N-tetraose or lacto-N-neotetraose.

9. Use of an enzyme of the glycoside hydrolase family 20 (GH20) for producing lacto-N-triose II or lacto-N-tetraose from lactose.

10. Use according to claim 9, wherein glucosamine-oxazoline and/or lacto-N-biose-oxazoline are used as substrates.

11. Use The use according to claim 9 or 10, wherein the enzyme of the glycoside hydrolase family 20 (GH20) is used to produce lacto-N-triose II and/or lacto-N -tetraose by reacting glucosamine-oxazoline and/or lacto-N-biose-oxazoline.

12. An enzyme, comprising β-N-acetylhexosaminidase having an amino acid sequence identity of at least 70% to SEQ ID NO: 1 and carrying at least one mutation selected from the group of mutations consisting of: a mutation at position 746 of SEQ ID NO: 1; a mutation at position 827 of SEQ ID NO: 1, preferably carrying a glutamic acid or an alanine or a glutamine; a mutation at position 746 of SEQ ID NO: 1 and/or carrying a phenylalanine at a position counted as position 827 of SEQ ID NO: 1, or a β-N-acetylhexosaminidase comprising an amino acid sequence selected from the group consisting of: any-oUSEQ ID NO[[s]]: 5 to 8 or comprising an amino acid sequence having an amino acid sequence identity of at least 70% to any of SEQ ID NOs: 5 to 8.

13. A Lacto-N-biosidase having an amino acid sequence identity of at least 70% to SEQ ID NO: 3 and carrying at least one of the following substitutes selected from the group consisting of: a mutation at a position counted as position 320 of SEQ ID NO: 3, a mutation at a position counted as position 419 of SEQ ID NO: 3, a glutamic acid or an alanine at a position counted as position 320 of SEQ ID NO: 3, a phenylalanine at a position counted as position 419 of SEQ ID NO: 3; or
At least one lacto-N-biosidase comprising an amino acid sequence selected from the group consisting of; SEQ ID NO: 9, SEQ ID NO:10, SEQ ID NO: 11.
and a sequence having an amino acid sequence identity of at least 70% to any of SEQ ID NO: 9, SEQ ID NO:10, SEQ ID NO: 11.

14. The method according to claim 1, wherein the enzyme used is:
a β-N-acetylhexosaminidase having an amino acid sequence identity of at least 70% to SEQ ID NO: and carrying at least one mutation selected from the group of mutations consisting of: a mutation at position 746 of SEQ ID NO: la mutation at position 827 of SEQ ID NO: 1, preferably carrying a glutamic acid or an alanine or a glutamine a mutation at a position 746 of SEQ ID NO: 1 and/or carrying a phenylalanine at a position counted as position 827 of SEQ ID NO: 1, or β-N-acetylhexosaminidase comprising an amino acid sequence selected from the group consisting of: SEQ ID No: 5 to 8 or comprising an amino acid sequence having an amino acid sequence identity of at least 70% to any of SEQ ID NOs: 5 to 8, or
a Lacto-N-biosidase having an amino acid sequence identity of at least 70% to SEQ ID NO: 3 and carrying at least one of the following substitutes selected from the group consisting of; a mutation at a position counted as position 320 of SEQ ID NO: 3, a mutation at a position counted as position 419 of SEQ ID NO: 3, a glutamic acid or an alanine at a position counted as position 320 of SEQ ID NO: 3, a phenylalanine at a position counted as position 419 of SEQ ID NO: 3; or
at least one lacto-N-biosidase comprising an amino acid sequence selected from the group consisting of SEQ ID NO: 9, SEQ ID NO:10, and SEQ ID NO: 11.

15. Aqueous solution comprising an enzyme of the glycoside hydrolase family 20 (GH20) as defined in any of the preceding claims, lactose, preferably at least 50 g/L lactose, more preferably at least 100 g/L lactose, particularly preferably at least 150 g/L lactose, most preferably at least 190 g/L lactose and
a) glucosamine-oxazoline and/or lacto-N-biose-oxazoline; and/or
b) lacto-N-triose II and/or lacto-N-tetraose.

16. The method according to claim 1, wherein the enzyme used is:
a β-N-acetylhexosaminidase having an amino acid sequence identity of at least 70% to SEQ ID NO: and carrying at least one mutation selected from the group of mutations consisting of: a mutation at position 746 of SEQ ID NO: 1a mutation at position 827 of SEQ ID NO: 1, preferably carrying a glutamic acid or an alanine or a glutamine; a mutation at position 746 of SEQ ID NO: 1 and/or carrying a phenylalanine at a position counted as position 827 of SEQ ID NO: 1, or a β-N-acetylhexosaminidase comprising an amino acid sequence selected from the group consisting of: SEQ ID NOs: 5 to 8 or comprising an amino acid sequence having an amino acid sequence identity of at least 70% to any of SEQ ID NOs: 5 to 8, or
a Lacto-N-biosidase having an amino acid sequence identity of at least 70% to SEQ ID NO: 3 and carrying at least one of the following substitutes selected from the group consisting of; a mutation at a position counted as position 320 of SEQ ID NO: 3, a mutation at a position counted as position 419 of SEQ ID NO: 3, a glutamic acid or an alanine at a position counted as position 320 of SEQ ID NO: 3, a phenylalanine at a position counted as position 419 of SEQ ID NO: 3; or
at least one lacto-N-biosidase comprising an amino acid sequence selected from the group consisting of SEQ ID NO: 9, SEQ ID NO:10, and SEQ ID NO: 11.


SEQUENCE LISTING (27 sequences)

SEQ ID NO: 1 - protein, 1627 aa, Bifidobacterium bifidum
MRLRRVKAAIGSVLAAVTLLSMSLTGVTAAQASDDNLALNQTVTASSYEVATTAPEKAVDGDLGTRWGTAQNKAANEWIEVGLGGTKTVKQINIDFERKDADQNITSFKVELKQGDTYTKVYQKDTRAKQQEIILLDQAQQASAVKVTVLSADGGTMNWVNVGINEISVYSAPKETVLDTADTNHMLGATMTASSNETATLTPDKAIDQNRTGRNNRWASGYETPSNIWLKAEFPRLTAVKDIRIYFFERDVNPKPTNVQSFDLSYTDSEGTEHTLKSGYAMTASGAGYVADVVIQLDQAVNARSLKLSNFAIKSSEYNNVSVAEWEAYSNDQAEPGATLDSVVSDLESNHLTIETDTDTLALPTVPDGYTVKFNGADYEQLIAADGTVNHPLVDKTVQVAYVVTDTATGNTKTTSDIPYVVKGTNQQQEGNNAKPTIIPEIAEWHSTSAAKLAASAVTKVVYDDDSLKAVVDEFVADYKDFTGIKLTAKKGAAEAGAFNFVKTDSTAAIAQLGDEGYTMDIRADRVVAKSSSVTGNMYAMQTILQMTKQDANGFVIGSMRDYPRFTTRGLLLDVARKPVSLEMMREITRTMRYYKMNDFQAHLSDNYIFLENYGKGDNEDEAFKAYDAFRLESSLTNDKGESPTAEDYSISKKTFKQFIQDERALGMNVVPEIDVPAHANSFTKIWPELMVKGRVSPINSNRPLIDHLDVSKPETIAKIKEIFDDYTKGDDPTFDSDTTVHIGADEFLYNYTAYRKFINEIVPYIKDTNTVRMWGGLTWINDHKTEITKDAIENVEMNLWSKDWADGLQMYNMGYKLINTIDDYGYMVPNGSYGRANAYGDLLNISRVFDSFEPNKVRSSGGYQAVPSGDDQMLGAAFAIWSDNIDKSASGLTESDLYWRFFDAMPFYAEKTWAATGKEKGTAAKLTALAAKQGTGPRTNPYYQATSKNSVYESYDFNDGLADASGNGRDLTIGDGSKAAVKDQSLKLAGGSSYATSKLDKLGNGNELTFDVTLQQAAKPGDILFEADAPYGTHDIRVMENGKLGFTRELYNYYFDYELPVGKTVTVTIKVDQQTTKLYVDGEFVSDATGKYIDKGIEKKTGITAATFALPLQRIGSKTSAINGVIDNVIVKKSEAETDQYNKSCWTGTTNSETQYNDTEGLLRYAFDNNPSTIWHSNWKGATDKLTGSNSFYAEIDMCQKYTINQFSFTPRTSQDSGQVTKADLYVKANANDEWKQVATDQVFEASRAKKTFMFDEQEVRYVKFVAKSSNDGWVAVSEFGVANKPSSTVRVFVAADPAEGGTVSVAAEGETGTDTAVDVASGASVTAKAVAADGYRFSGWFTTASETAVSTDATYTFAADGNTALTAKFTKDSTPDPGPKPTISSIAVTKPTVTDYKVGDTFDATGLAVTATMSDGSTKTLTAGEYTLSATQDGAAVALDKAFAKAGKVTVTVTANGKTATFDVTVTAKDPDPEPATLKSIKVTSKPDKTTYTVDETFAKTGLAVTGTWSDGKTALLKDGEYKLSAVDADGKTVDLTKPFTAAGDVTVTVTSGKLTDSFTITVKAKTVTPAPGDNKPGENKPGADKPKPNTPDEVAKTGASVTAVVFSALLLLSAGYLLVRKRRI

SEQ ID NO: 2 - protein, 1567 aa, Bifidobacterium bifidum
SDDNLALNQTVTASSYEVATTAPEKAVDGDLGTRWGTAQNKAANEWIEVGLGGTKTVKQINIDFERKDADQNITSFKVELKQGDTYTKVYQKDTRAKQQEIILLDQAQQASAVKVTVLSADGGTMNWVNVGINEISVYSAPKETVLDTADTNHMLGATMTASSNETATLTPDKAIDQNRTGRNNRWASGYETPSNIWLKAEFPRLTAVKDIRIYFFERDVNPKPTNVQSFDLSYTDSEGTEHTLKSGYAMTASGAGYVADVVIQLDQAVNARSLKLSNFAIKSSEYNNVSVAEWEAYSNDQAEPGATLDSVVSDLESNHLTIETDTDTLALPTVPDGYTVKFNGADYEQLIAADGTVNHPLVDKTVQVAYVVTDTATGNTKTTSDIPYVVKGTNQQQEGNNAKPTIIPEIAEWHSTSAAKLAASAVTKVVYDDDSLKAVVDEFVADYKDFTGIKLTAKKGAAEAGAFNFVKTDSTAAIAQLGDEGYTMDIRADRVVAKSSSVTGNMYAMQTILQMTKQDANGFVIGSMRDYPRFTTRGLLLDVARKPVSLEMMREITRTMRYYKMNDFQAHLSDNYIFLENYGKGDNEDEAFKAYDAFRLESSLTNDKGESPTAEDYSISKKTFKQFIQDERALGMNVVPEIDVPAHANSFTKIWPELMVKGRVSPINSNRPLIDHLDVSKPETIAKIKEIFDDYTKGDDPTFDSDTTVHIGADEFLYNYTAYRKFINEIVPYIKDTNTVRMWGGLTWINDHKTEITKDAIENVEMNLWSKDWADGLQMYNMGYKLINTIDDYGYMVPNGSYGRANAYGDLLNISRVFDSFEPNKVRSSGGYQAVPSGDDQMLGAAFAIWSDNIDKSASGLTESDLYWRFFDAMPFYAEKTWAATGKEKGTAAKLTALAAKQGTGPRTNPYYQATSKNSVYESYDFNDGLADASGNGRDLTIGDGSKAAVKDQSLKLAGGSSYATSKLDKLGNGNELTFDVTLQQAAKPGDILFEADAPYGTHDIRVMENGKLGFTRELYNYYFDYELPVGKTVTVTIKVDQQTTKLYVDGEFVSDATGKYIDKGIEKKTGITAATFALPLQRIGSKTSAINGVIDNVIVKKSEAETDQYNKSCWTGTTNSETQYNDTEGLLRYAFDNNPSTIWHSNWKGATDKLTGSNSFYAEIDMCQKYTINQFSFTPRTSQDSGQVTKADLYVKANANDEWKQVATDQVFEASRAKKTFMFDEQEVRYVKFVAKSSNDGWVAVSEFGVANKPSSTVRVFVAADPAEGGTVSVAAEGETGTDTAVDVASGASVTAKAVAADGYRFSGWFTTASETAVSTDATYTFAADGNTALTAKFTKDSTPDPGPKPTISSIAVTKPTVTDYKVGDTFDATGLAVTATMSDGSTKTLTAGEYTLSATQDGAAVALDKAFAKAGKVTVTVTANGKTATFDVTVTAKDPDPEPATLKSIKVTSKPDKTTYTVDETFAKTGLAVTGTWSDGKTALLKDGEYKLSAVDADGKTVDLTKPFTAAGDVTVTVTSGKLTDSFTITVKAKTVTPAPGDNKPGENKPGADKPKPNTPDEVAK

SEQ ID NO: 3 - protein, 1112 aa, Bifidobacterium bifidum
MEKSSNRRFGVRTVAAIVAGLMVGGMCTAMTASAADDSAAGYSATAPVNLTRPATVPSMDGWTDGTGAWTLGEGTRVVSSDALAARAQSLASELTKFTDVDIKAATGSATGKDISLTLDASKKAELGDEGFKLNIGSKGLEVIGATDIGVFYGTRSVSQMLRQGQLTLPAGTVATKPKYKERGATLCACQINISTDWIDRFLSDMADLRLNYVLLEMKLKPEEDNTKKAATWSYYTRDDVKKFVKKANNYGIDVIPEINSPGHMNVWLENYPEYQLADNSGRKDPNKLDISNPEAVKFYKTLIDEYDGVFTTKYWHMGADEYMIGTSFDNYSKLKTFAEKQYGAGATPNDAFTGFINDIDKYVKAKGKQLRIWNDGIVNTKNVSLNKDIVIEYWYGAGRKPQELVQDGYTLMNATQALYWSRSAQVYKVNAARLYNNNWNVGTFDGGRQIDKNYDKLTGAKVSIWPDSSYFQTENEVEKEIFDGMRFISQMTWSDSRPWATWNDMKADIDKIGYPLDIREYDYTPVDAGIYDIPQLKSISKGPWELITTPDGYYQMKDTVSGKCLALFTGSKHLDVVTQVGARPELRNCADVSVGQDQRNTANERNTQKWQIRADKDGKYTISPALTQQRLAIATGNEQNIDLETHRPAAGTVAQFPADLVSDNALFTLTGHMGMSATVDSKTVNPASPSKITVKVRAASNANTGDVTVTPVVPEGWEIKPGSVSLKSIPAGKAAIAYFNVVNTTGTGDATVQFKLTNTKTGEELGTTSVALTGSLTKDVEASDYAASSQETTGEHAPVGNAFDKNANTFWHSKYSNPSANLPHWLAFKASPGEGNKIAAITHLYRQDKLNGPAKNVAVYVVAASDANSVADVTNWGEPVATAEFPYTKELQTIALPNTIPSGDVYVKFQINDAWGLTETSAGVTWAAVAELAATAKATPVELTEPEQPKDNPEVTETPEATGVTVSGDGVANGALSLKKGTTAQLTAKVAPDDADQAVTWASSDDKVVTVDKTGKVTAVAKGVAKVTATTANGKSASVTVTVTEDSEVPGPTGPTEPTKPGTEKPTTKPTTKPNDGKLSATGADTAVLATIAALFALAGGAVVAVRRRSVR

SEQ ID NO: 4 - protein, 1030 aa, Bifidobacterium bifidum
ADDSAAGYSATAPVNLTRPATVPSMDGWTDGTGAWTLGEGTRVVSSDALAARAQSLASELTKFTDVDIKAATGSATGKDISLTLDASKKAELGDEGFKLNIGSKGLEVIGATDIGVFYGTRSVSQMLRQGQLTLPAGTVATKPKYKERGATLCACQINISTDWIDRFLSDMADLRLNYVLLEMKLKPEEDNTKKAATWSYYTRDDVKKFVKKANNYGIDVIPEINSPGHMNVWLENYPEYQLADNSGRKDPNKLDISNPEAVKFYKTLIDEYDGVFTTKYWHMGADEYMIGTSFDNYSKLKTFAEKQYGAGATPNDAFTGFINDIDKYVKAKGKQLRIWNDGIVNTKNVSLNKDIVIEYWYGAGRKPQELVQDGYTLMNATQALYWSRSAQVYKVNAARLYNNNWNVGTFDGGRQIDKNYDKLTGAKVSIWPDSSYFQTENEVEKEIFDGMRFISQMTWSDSRPWATWNDMKADIDKIGYPLDIREYDYTPVDAGIYDIPQLKSISKGPWELITTPDGYYQMKDTVSGKCLALFTGSKHLDVVTQVGARPELRNCADVSVGQDQRNTANERNTQKWQIRADKDGKYTISPALTQQRLAIATGNEQNIDLETHRPAAGTVAQFPADLVSDNALFTLTGHMGMSATVDSKTVNPASPSKITVKVRAASNANTGDVTVTPVVPEGWEIKPGSVSLKSIPAGKAAIAYFNVVNTTGTGDATVQFKLTNTKTGEELGTTSVALTGSLTKDVEASDYAASSQETTGEHAPVGNAFDKNANTFWHSKYSNPSANLPHWLAFKASPGEGNKIAAITHLYRQDKLNGPAKNVAVYVVAASDANSVADVTNWGEPVATAEFPYTKELQTIALPNTIPSGDVYVKFQINDAWGLTETSAGVTWAAVAELAATAKATPVELTEPEQPKDNPEVTETPEATGVTVSGDGVANGALSLKKGTTAQLTAKVAPDDADQAVTWASSDDKVVTVDKTGKVTAVAKGVAKVTATTANGKSASVTVTVTEDSEVPGPTGPTEPTKPGTE

SEQ ID NO: 5 - protein, 1567 aa, Artificial Sequence
  Synthetic polypeptide (truncated version of a mutated Bbhl)
SDDNLALNQTVTASSYEVATTAPEKAVDGDLGTRWGTAQNKAANEWIEVGLGGTKTVKQINIDFERKDADQNITSFKVELKQGDTYTKVYQKDTRAKQQEIILLDQAQQASAVKVTVLSADGGTMNWVNVGINEISVYSAPKETVLDTADTNHMLGATMTASSNETATLTPDKAIDQNRTGRNNRWASGYETPSNIWLKAEFPRLTAVKDIRIYFFERDVNPKPTNVQSFDLSYTDSEGTEHTLKSGYAMTASGAGYVADVVIQLDQAVNARSLKLSNFAIKSSEYNNVSVAEWEAYSNDQAEPGATLDSVVSDLESNHLTIETDTDTLALPTVPDGYTVKFNGADYEQLIAADGTVNHPLVDKTVQVAYVVTDTATGNTKTTSDIPYVVKGTNQQQEGNNAKPTIIPEIAEWHSTSAAKLAASAVTKVVYDDDSLKAVVDEFVADYKDFTGIKLTAKKGAAEAGAFNFVKTDSTAAIAQLGDEGYTMDIRADRVVAKSSSVTGNMYAMQTILQMTKQDANGFVIGSMRDYPRFTTRGLLLDVARKPVSLEMMREITRTMRYYKMNDFQAHLSDNYIFLENYGKGDNEDEAFKAYDAFRLESSLTNDKGESPTAEDYSISKKTFKQFIQDERALGMNVVPEIDVPAHANSFTKIWPELMVKGRVSPINSNRPLIDHLDVSKPETIAKIKEIFDDYTKGDDPTFDSDTTVHIGAEEFLYNYTAYRKFINEIVPYIKDTNTVRMWGGLTWINDHKTEITKDAIENVEMNLWSKDWADGLQMYNMGYKLINTIDDYGYMVPNGSYGRANAYGDLLNISRVFDSFEPNKVRSSGGYQAVPSGDDQMLGAAFAIWSDNIDKSASGLTESDLYWRFFDAMPFYAEKTWAATGKEKGTAAKLTALAAKQGTGPRTNPYYQATSKNSVYESYDFNDGLADASGNGRDLTIGDGSKAAVKDQSLKLAGGSSYATSKLDKLGNGNELTFDVTLQQAAKPGDILFEADAPYGTHDIRVMENGKLGFTRELYNYYFDYELPVGKTVTVTIKVDQQTTKLYVDGEFVSDATGKYIDKGIEKKTGITAATFALPLQRIGSKTSAINGVIDNVIVKKSEAETDQYNKSCWTGTTNSETQYNDTEGLLRYAFDNNPSTIWHSNWKGATDKLTGSNSFYAEIDMCQKYTINQFSFTPRTSQDSGQVTKADLYVKANANDEWKQVATDQVFEASRAKKTFMFDEQEVRYVKFVAKSSNDGWVAVSEFGVANKPSSTVRVFVAADPAEGGTVSVAAEGETGTDTAVDVASGASVTAKAVAADGYRFSGWFTTASETAVSTDATYTFAADGNTALTAKFTKDSTPDPGPKPTISSIAVTKPTVTDYKVGDTFDATGLAVTATMSDGSTKTLTAGEYTLSATQDGAAVALDKAFAKAGKVTVTVTANGKTATFDVTVTAKDPDPEPATLKSIKVTSKPDKTTYTVDETFAKTGLAVTGTWSDGKTALLKDGEYKLSAVDADGKTVDLTKPFTAAGDVTVTVTSGKLTDSFTITVKAKTVTPAPGDNKPGENKPGADKPKPNTPDEVAK

SEQ ID NO: 6 - protein, 1567 aa, Artificial Sequence
  Synthetic polypeptide (truncated version of a mutated Bbhl)
SDDNLALNQTVTASSYEVATTAPEKAVDGDLGTRWGTAQNKAANEWIEVGLGGTKTVKQINIDFERKDADQNITSFKVELKQGDTYTKVYQKDTRAKQQEIILLDQAQQASAVKVTVLSADGGTMNWVNVGINEISVYSAPKETVLDTADTNHMLGATMTASSNETATLTPDKAIDQNRTGRNNRWASGYETPSNIWLKAEFPRLTAVKDIRIYFFERDVNPKPTNVQSFDLSYTDSEGTEHTLKSGYAMTASGAGYVADVVIQLDQAVNARSLKLSNFAIKSSEYNNVSVAEWEAYSNDQAEPGATLDSVVSDLESNHLTIETDTDTLALPTVPDGYTVKFNGADYEQLIAADGTVNHPLVDKTVQVAYVVTDTATGNTKTTSDIPYVVKGTNQQQEGNNAKPTIIPEIAEWHSTSAAKLAASAVTKVVYDDDSLKAVVDEFVADYKDFTGIKLTAKKGAAEAGAFNFVKTDSTAAIAQLGDEGYTMDIRADRVVAKSSSVTGNMYAMQTILQMTKQDANGFVIGSMRDYPRFTTRGLLLDVARKPVSLEMMREITRTMRYYKMNDFQAHLSDNYIFLENYGKGDNEDEAFKAYDAFRLESSLTNDKGESPTAEDYSISKKTFKQFIQDERALGMNVVPEIDVPAHANSFTKIWPELMVKGRVSPINSNRPLIDHLDVSKPETIAKIKEIFDDYTKGDDPTFDSDTTVHIGAAEFLYNYTAYRKFINEIVPYIKDTNTVRMWGGLTWINDHKTEITKDAIENVEMNLWSKDWADGLQMYNMGYKLINTIDDYGYMVPNGSYGRANAYGDLLNISRVFDSFEPNKVRSSGGYQAVPSGDDQMLGAAFAIWSDNIDKSASGLTESDLYWRFFDAMPFYAEKTWAATGKEKGTAAKLTALAAKQGTGPRTNPYYQATSKNSVYESYDFNDGLADASGNGRDLTIGDGSKAAVKDQSLKLAGGSSYATSKLDKLGNGNELTFDVTLQQAAKPGDILFEADAPYGTHDIRVMENGKLGFTRELYNYYFDYELPVGKTVTVTIKVDQQTTKLYVDGEFVSDATGKYIDKGIEKKTGITAATFALPLQRIGSKTSAINGVIDNVIVKKSEAETDQYNKSCWTGTTNSETQYNDTEGLLRYAFDNNPSTIWHSNWKGATDKLTGSNSFYAEIDMCQKYTINQFSFTPRTSQDSGQVTKADLYVKANANDEWKQVATDQVFEASRAKKTFMFDEQEVRYVKFVAKSSNDGWVAVSEFGVANKPSSTVRVFVAADPAEGGTVSVAAEGETGTDTAVDVASGASVTAKAVAADGYRFSGWFTTASETAVSTDATYTFAADGNTALTAKFTKDSTPDPGPKPTISSIAVTKPTVTDYKVGDTFDATGLAVTATMSDGSTKTLTAGEYTLSATQDGAAVALDKAFAKAGKVTVTVTANGKTATFDVTVTAKDPDPEPATLKSIKVTSKPDKTTYTVDETFAKTGLAVTGTWSDGKTALLKDGEYKLSAVDADGKTVDLTKPFTAAGDVTVTVTSGKLTDSFTITVKAKTVTPAPGDNKPGENKPGADKPKPNTPDEVAK

SEQ ID NO: 7 - protein, 1567 aa, Artificial Sequence
  Synthetic polypeptide (truncated version of a mutated Bbhl)
SDDNLALNQTVTASSYEVATTAPEKAVDGDLGTRWGTAQNKAANEWIEVGLGGTKTVKQINIDFERKDADQNITSFKVELKQGDTYTKVYQKDTRAKQQEIILLDQAQQASAVKVTVLSADGGTMNWVNVGINEISVYSAPKETVLDTADTNHMLGATMTASSNETATLTPDKAIDQNRTGRNNRWASGYETPSNIWLKAEFPRLTAVKDIRIYFFERDVNPKPTNVQSFDLSYTDSEGTEHTLKSGYAMTASGAGYVADVVIQLDQAVNARSLKLSNFAIKSSEYNNVSVAEWEAYSNDQAEPGATLDSVVSDLESNHLTIETDTDTLALPTVPDGYTVKFNGADYEQLIAADGTVNHPLVDKTVQVAYVVTDTATGNTKTTSDIPYVVKGTNQQQEGNNAKPTIIPEIAEWHSTSAAKLAASAVTKVVYDDDSLKAVVDEFVADYKDFTGIKLTAKKGAAEAGAFNFVKTDSTAAIAQLGDEGYTMDIRADRVVAKSSSVTGNMYAMQTILQMTKQDANGFVIGSMRDYPRFTTRGLLLDVARKPVSLEMMREITRTMRYYKMNDFQAHLSDNYIFLENYGKGDNEDEAFKAYDAFRLESSLTNDKGESPTAEDYSISKKTFKQFIQDERALGMNVVPEIDVPAHANSFTKIWPELMVKGRVSPINSNRPLIDHLDVSKPETIAKIKEIFDDYTKGDDPTFDSDTTVHIGAQEFLYNYTAYRKFINEIVPYIKDTNTVRMWGGLTWINDHKTEITKDAIENVEMNLWSKDWADGLQMYNMGYKLINTIDDYGYMVPNGSYGRANAYGDLLNISRVFDSFEPNKVRSSGGYQAVPSGDDQMLGAAFAIWSDNIDKSASGLTESDLYWRFFDAMPFYAEKTWAATGKEKGTAAKLTALAAKQGTGPRTNPYYQATSKNSVYESYDFNDGLADASGNGRDLTIGDGSKAAVKDQSLKLAGGSSYATSKLDKLGNGNELTFDVTLQQAAKPGDILFEADAPYGTHDIRVMENGKLGFTRELYNYYFDYELPVGKTVTVTIKVDQQTTKLYVDGEFVSDATGKYIDKGIEKKTGITAATFALPLQRIGSKTSAINGVIDNVIVKKSEAETDQYNKSCWTGTTNSETQYNDTEGLLRYAFDNNPSTIWHSNWKGATDKLTGSNSFYAEIDMCQKYTINQFSFTPRTSQDSGQVTKADLYVKANANDEWKQVATDQVFEASRAKKTFMFDEQEVRYVKFVAKSSNDGWVAVSEFGVANKPSSTVRVFVAADPAEGGTVSVAAEGETGTDTAVDVASGASVTAKAVAADGYRFSGWFTTASETAVSTDATYTFAADGNTALTAKFTKDSTPDPGPKPTISSIAVTKPTVTDYKVGDTFDATGLAVTATMSDGSTKTLTAGEYTLSATQDGAAVALDKAFAKAGKVTVTVTANGKTATFDVTVTAKDPDPEPATLKSIKVTSKPDKTTYTVDETFAKTGLAVTGTWSDGKTALLKDGEYKLSAVDADGKTVDLTKPFTAAGDVTVTVTSGKLTDSFTITVKAKTVTPAPGDNKPGENKPGADKPKPNTPDEVAK

SEQ ID NO: 8 - protein, 1567 aa, Artificial Sequence
  Synthetic polypeptide (truncated version of a mutated Bbhl)
SDDNLALNQTVTASSYEVATTAPEKAVDGDLGTRWGTAQNKAANEWIEVGLGGTKTVKQINIDFERKDADQNITSFKVELKQGDTYTKVYQKDTRAKQQEIILLDQAQQASAVKVTVLSADGGTMNWVNVGINEISVYSAPKETVLDTADTNHMLGATMTASSNETATLTPDKAIDQNRTGRNNRWASGYETPSNIWLKAEFPRLTAVKDIRIYFFERDVNPKPTNVQSFDLSYTDSEGTEHTLKSGYAMTASGAGYVADVVIQLDQAVNARSLKLSNFAIKSSEYNNVSVAEWEAYSNDQAEPGATLDSVVSDLESNHLTIETDTDTLALPTVPDGYTVKFNGADYEQLIAADGTVNHPLVDKTVQVAYVVTDTATGNTKTTSDIPYVVKGTNQQQEGNNAKPTIIPEIAEWHSTSAAKLAASAVTKVVYDDDSLKAVVDEFVADYKDFTGIKLTAKKGAAEAGAFNFVKTDSTAAIAQLGDEGYTMDIRADRVVAKSSSVTGNMYAMQTILQMTKQDANGFVIGSMRDYPRFTTRGLLLDVARKPVSLEMMREITRTMRYYKMNDFQAHLSDNYIFLENYGKGDNEDEAFKAYDAFRLESSLTNDKGESPTAEDYSISKKTFKQFIQDERALGMNVVPEIDVPAHANSFTKIWPELMVKGRVSPINSNRPLIDHLDVSKPETIAKIKEIFDDYTKGDDPTFDSDTTVHIGADEFLYNYTAYRKFINEIVPYIKDTNTVRMWGGLTWINDHKTEITKDAIENVEMNLWSKDWADGLQMYNMGYKLINTIDDYGFMVPNGSYGRANAYGDLLNISRVFDSFEPNKVRSSGGYQAVPSGDDQMLGAAFAIWSDNIDKSASGLTESDLYWRFFDAMPFYAEKTWAATGKEKGTAAKLTALAAKQGTGPRTNPYYQATSKNSVYESYDFNDGLADASGNGRDLTIGDGSKAAVKDQSLKLAGGSSYATSKLDKLGNGNELTFDVTLQQAAKPGDILFEADAPYGTHDIRVMENGKLGFTRELYNYYFDYELPVGKTVTVTIKVDQQTTKLYVDGEFVSDATGKYIDKGIEKKTGITAATFALPLQRIGSKTSAINGVIDNVIVKKSEAETDQYNKSCWTGTTNSETQYNDTEGLLRYAFDNNPSTIWHSNWKGATDKLTGSNSFYAEIDMCQKYTINQFSFTPRTSQDSGQVTKADLYVKANANDEWKQVATDQVFEASRAKKTFMFDEQEVRYVKFVAKSSNDGWVAVSEFGVANKPSSTVRVFVAADPAEGGTVSVAAEGETGTDTAVDVASGASVTAKAVAADGYRFSGWFTTASETAVSTDATYTFAADGNTALTAKFTKDSTPDPGPKPTISSIAVTKPTVTDYKVGDTFDATGLAVTATMSDGSTKTLTAGEYTLSATQDGAAVALDKAFAKAGKVTVTVTANGKTATFDVTVTAKDPDPEPATLKSIKVTSKPDKTTYTVDETFAKTGLAVTGTWSDGKTALLKDGEYKLSAVDADGKTVDLTKPFTAAGDVTVTVTSGKLTDSFTITVKAKTVTPAPGDNKPGENKPGADKPKPNTPDEVAK

SEQ ID NO: 9 - protein, 1030 aa, Artificial Sequence
  Synthetic polypeptide (mutated lacto-N- biosidase)
ADDSAAGYSATAPVNLTRPATVPSMDGWTDGTGAWTLGEGTRVVSSDALAARAQSLASELTKFTDVDIKAATGSATGKDISLTLDASKKAELGDEGFKLNIGSKGLEVIGATDIGVFYGTRSVSQMLRQGQLTLPAGTVATKPKYKERGATLCACQINISTDWIDRFLSDMADLRLNYVLLEMKLKPEEDNTKKAATWSYYTRDDVKKFVKKANNYGIDVIPEINSPGHMNVWLENYPEYQLADNSGRKDPNKLDISNPEAVKFYKTLIDEYDGVFTTKYWHMGAEEYMIGTSFDNYSKLKTFAEKQYGAGATPNDAFTGFINDIDKYVKAKGKQLRIWNDGIVNTKNVSLNKDIVIEYWYGAGRKPQELVQDGYTLMNATQALYWSRSAQVYKVNAARLYNNNWNVGTFDGGRQIDKNYDKLTGAKVSIWPDSSYFQTENEVEKEIFDGMRFISQMTWSDSRPWATWNDMKADIDKIGYPLDIREYDYTPVDAGIYDIPQLKSISKGPWELITTPDGYYQMKDTVSGKCLALFTGSKHLDVVTQVGARPELRNCADVSVGQDQRNTANERNTQKWQIRADKDGKYTISPALTQQRLAIATGNEQNIDLETHRPAAGTVAQFPADLVSDNALFTLTGHMGMSATVDSKTVNPASPSKITVKVRAASNANTGDVTVTPVVPEGWEIKPGSVSLKSIPAGKAAIAYFNVVNTTGTGDATVQFKLTNTKTGEELGTTSVALTGSLTKDVEASDYAASSQETTGEHAPVGNAFDKNANTFWHSKYSNPSANLPHWLAFKASPGEGNKIAAITHLYRQDKLNGPAKNVAVYVVAASDANSVADVTNWGEPVATAEFPYTKELQTIALPNTIPSGDVYVKFQINDAWGLTETSAGVTWAAVAELAATAKATPVELTEPEQPKDNPEVTETPEATGVTVSGDGVANGALSLKKGTTAQLTAKVAPDDADQAVTWASSDDKVVTVDKTGKVTAVAKGVAKVTATTANGKSASVTVTVTEDSEVPGPTGPTEPTKPGTE

SEQ ID NO: 10 - protein, 1030 aa, Artificial Sequence
  Synthetic polypeptide (mutated lacto-N- biosidase)
ADDSAAGYSATAPVNLTRPATVPSMDGWTDGTGAWTLGEGTRVVSSDALAARAQSLASELTKFTDVDIKAATGSATGKDISLTLDASKKAELGDEGFKLNIGSKGLEVIGATDIGVFYGTRSVSQMLRQGQLTLPAGTVATKPKYKERGATLCACQINISTDWIDRFLSDMADLRLNYVLLEMKLKPEEDNTKKAATWSYYTRDDVKKFVKKANNYGIDVIPEINSPGHMNVWLENYPEYQLADNSGRKDPNKLDISNPEAVKFYKTLIDEYDGVFTTKYWHMGAAEYMIGTSFDNYSKLKTFAEKQYGAGATPNDAFTGFINDIDKYVKAKGKQLRIWNDGIVNTKNVSLNKDIVIEYWYGAGRKPQELVQDGYTLMNATQALYWSRSAQVYKVNAARLYNNNWNVGTFDGGRQIDKNYDKLTGAKVSIWPDSSYFQTENEVEKEIFDGMRFISQMTWSDSRPWATWNDMKADIDKIGYPLDIREYDYTPVDAGIYDIPQLKSISKGPWELITTPDGYYQMKDTVSGKCLALFTGSKHLDVVTQVGARPELRNCADVSVGQDQRNTANERNTQKWQIRADKDGKYTISPALTQQRLAIATGNEQNIDLETHRPAAGTVAQFPADLVSDNALFTLTGHMGMSATVDSKTVNPASPSKITVKVRAASNANTGDVTVTPVVPEGWEIKPGSVSLKSIPAGKAAIAYFNVVNTTGTGDATVQFKLTNTKTGEELGTTSVALTGSLTKDVEASDYAASSQETTGEHAPVGNAFDKNANTFWHSKYSNPSANLPHWLAFKASPGEGNKIAAITHLYRQDKLNGPAKNVAVYVVAASDANSVADVTNWGEPVATAEFPYTKELQTIALPNTIPSGDVYVKFQINDAWGLTETSAGVTWAAVAELAATAKATPVELTEPEQPKDNPEVTETPEATGVTVSGDGVANGALSLKKGTTAQLTAKVAPDDADQAVTWASSDDKVVTVDKTGKVTAVAKGVAKVTATTANGKSASVTVTVTEDSEVPGPTGPTEPTKPGTE

SEQ ID NO: 11 - protein, 1030 aa, Artificial Sequence
  Synthetic polypeptide (mutated lacto-N- biosidase)
ADDSAAGYSATAPVNLTRPATVPSMDGWTDGTGAWTLGEGTRVVSSDALAARAQSLASELTKFTDVDIKAATGSATGKDISLTLDASKKAELGDEGFKLNIGSKGLEVIGATDIGVFYGTRSVSQMLRQGQLTLPAGTVATKPKYKERGATLCACQINISTDWIDRFLSDMADLRLNYVLLEMKLKPEEDNTKKAATWSYYTRDDVKKFVKKANNYGIDVIPEINSPGHMNVWLENYPEYQLADNSGRKDPNKLDISNPEAVKFYKTLIDEYDGVFTTKYWHMGADEYMIGTSFDNYSKLKTFAEKQYGAGATPNDAFTGFINDIDKYVKAKGKQLRIWNDGIVNTKNVSLNKDIVIEYWYGAGRKPQELVQDGYTLMNATQALFWSRSAQVYKVNAARLYNNNWNVGTFDGGRQIDKNYDKLTGAKVSIWPDSSYFQTENEVEKEIFDGMRFISQMTWSDSRPWATWNDMKADIDKIGYPLDIREYDYTPVDAGIYDIPQLKSISKGPWELITTPDGYYQMKDTVSGKCLALFTGSKHLDVVTQVGARPELRNCADVSVGQDQRNTANERNTQKWQIRADKDGKYTISPALTQQRLAIATGNEQNIDLETHRPAAGTVAQFPADLVSDNALFTLTGHMGMSATVDSKTVNPASPSKITVKVRAASNANTGDVTVTPVVPEGWEIKPGSVSLKSIPAGKAAIAYFNVVNTTGTGDATVQFKLTNTKTGEELGTTSVALTGSLTKDVEASDYAASSQETTGEHAPVGNAFDKNANTFWHSKYSNPSANLPHWLAFKASPGEGNKIAAITHLYRQDKLNGPAKNVAVYVVAASDANSVADVTNWGEPVATAEFPYTKELQTIALPNTIPSGDVYVKFQINDAWGLTETSAGVTWAAVAELAATAKATPVELTEPEQPKDNPEVTETPEATGVTVSGDGVANGALSLKKGTTAQLTAKVAPDDADQAVTWASSDDKVVTVDKTGKVTAVAKGVAKVTATTANGKSASVTVTVTEDSEVPGPTGPTEPTKPGTE

SEQ ID NO: 12 - protein, 1597 aa, Artificial Sequence
  Synthetic polypeptide (BbhI, wild type (wt), truncated construct including a His-tag)
MASMTGGQQMGRDPNSSSVDKLSDDNLALNQTVTASSYEVATTAPEKAVDGDLGTRWGTAQNKAANEWIEVGLGGTKTVKQINIDFERKDADQNITSFKVELKQGDTYTKVYQKDTRAKQQEIILLDQAQQASAVKVTVLSADGGTMNWVNVGINEISVYSAPKETVLDTADTNHMLGATMTASSNETATLTPDKAIDQNRTGRNNRWASGYETPSNIWLKAEFPRLTAVKDIRIYFFERDVNPKPTNVQSFDLSYTDSEGTEHTLKSGYAMTASGAGYVADVVIQLDQAVNARSLKLSNFAIKSSEYNNVSVAEWEAYSNDQAEPGATLDSVVSDLESNHLTIETDTDTLALPTVPDGYTVKFNGADYEQLIAADGTVNHPLVDKTVQVAYVVTDTATGNTKTTSDIPYVVKGTNQQQEGNNAKPTIIPEIAEWHSTSAAKLAASAVTKVVYDDDSLKAVVDEFVADYKDFTGIKLTAKKGAAEAGAFNFVKTDSTAAIAQLGDEGYTMDIRADRVVAKSSSVTGNMYAMQTILQMTKQDANGFVIGSMRDYPRFTTRGLLLDVARKPVSLEMMREITRTMRYYKMNDFQAHLSDNYIFLENYGKGDNEDEAFKAYDAFRLESSLTNDKGESPTAEDYSISKKTFKQFIQDERALGMNVVPEIDVPAHANSFTKIWPELMVKGRVSPINSNRPLIDHLDVSKPETIAKIKEIFDDYTKGDDPTFDSDTTVHIGADEFLYNYTAYRKFINEIVPYIKDTNTVRMWGGLTWINDHKTEITKDAIENVEMNLWSKDWADGLQMYNMGYKLINTIDDYGYMVPNGSYGRANAYGDLLNISRVFDSFEPNKVRSSGGYQAVPSGDDQMLGAAFAIWSDNIDKSASGLTESDLYWRFFDAMPFYAEKTWAATGKEKGTAAKLTALAAKQGTGPRTNPYYQATSKNSVYESYDFNDGLADASGNGRDLTIGDGSKAAVKDQSLKLAGGSSYATSKLDKLGNGNELTFDVTLQQAAKPGDILFEADAPYGTHDIRVMENGKLGFTRELYNYYFDYELPVGKTVTVTIKVDQQTTKLYVDGEFVSDATGKYIDKGIEKKTGITAATFALPLQRIGSKTSAINGVIDNVIVKKSEAETDQYNKSCWTGTTNSETQYNDTEGLLRYAFDNNPSTIWHSNWKGATDKLTGSNSFYAEIDMCQKYTINQFSFTPRTSQDSGQVTKADLYVKANANDEWKQVATDQVFEASRAKKTFMFDEQEVRYVKFVAKSSNDGWVAVSEFGVANKPSSTVRVFVAADPAEGGTVSVAAEGETGTDTAVDVASGASVTAKAVAADGYRFSGWFTTASETAVSTDATYTFAADGNTALTAKFTKDSTPDPGPKPTISSIAVTKPTVTDYKVGDTFDATGLAVTATMSDGSTKTLTAGEYTLSATQDGAAVALDKAFAKAGKVTVTVTANGKTATFDVTVTAKDPDPEPATLKSIKVTSKPDKTTYTVDETFAKTGLAVTGTWSDGKTALLKDGEYKLSAVDADGKTVDLTKPFTAAGDVTVTVTSGKLTDSFTITVKAKTVTPAPGDNKPGENKPGADKPKPNTPDEVAKLEHHHHHH

SEQ ID NO: 13 - protein, 1597 aa, Artificial Sequence
  Synthetic polypeptide (BbhI, D746E mutant, truncated construct including a His-tag)
MASMTGGQQMGRDPNSSSVDKLSDDNLALNQTVTASSYEVATTAPEKAVDGDLGTRWGTAQNKAANEWIEVGLGGTKTVKQINIDFERKDADQNITSFKVELKQGDTYTKVYQKDTRAKQQEIILLDQAQQASAVKVTVLSADGGTMNWVNVGINEISVYSAPKETVLDTADTNHMLGATMTASSNETATLTPDKAIDQNRTGRNNRWASGYETPSNIWLKAEFPRLTAVKDIRIYFFERDVNPKPTNVQSFDLSYTDSEGTEHTLKSGYAMTASGAGYVADVVIQLDQAVNARSLKLSNFAIKSSEYNNVSVAEWEAYSNDQAEPGATLDSVVSDLESNHLTIETDTDTLALPTVPDGYTVKFNGADYEQLIAADGTVNHPLVDKTVQVAYVVTDTATGNTKTTSDIPYVVKGTNQQQEGNNAKPTIIPEIAEWHSTSAAKLAASAVTKVVYDDDSLKAVVDEFVADYKDFTGIKLTAKKGAAEAGAFNFVKTDSTAAIAQLGDEGYTMDIRADRVVAKSSSVTGNMYAMQTILQMTKQDANGFVIGSMRDYPRFTTRGLLLDVARKPVSLEMMREITRTMRYYKMNDFQAHLSDNYIFLENYGKGDNEDEAFKAYDAFRLESSLTNDKGESPTAEDYSISKKTFKQFIQDERALGMNVVPEIDVPAHANSFTKIWPELMVKGRVSPINSNRPLIDHLDVSKPETIAKIKEIFDDYTKGDDPTFDSDTTVHIGAEEFLYNYTAYRKFINEIVPYIKDTNTVRMWGGLTWINDHKTEITKDAIENVEMNLWSKDWADGLQMYNMGYKLINTIDDYGYMVPNGSYGRANAYGDLLNISRVFDSFEPNKVRSSGGYQAVPSGDDQMLGAAFAIWSDNIDKSASGLTESDLYWRFFDAMPFYAEKTWAATGKEKGTAAKLTALAAKQGTGPRTNPYYQATSKNSVYESYDFNDGLADASGNGRDLTIGDGSKAAVKDQSLKLAGGSSYATSKLDKLGNGNELTFDVTLQQAAKPGDILFEADAPYGTHDIRVMENGKLGFTRELYNYYFDYELPVGKTVTVTIKVDQQTTKLYVDGEFVSDATGKYIDKGIEKKTGITAATFALPLQRIGSKTSAINGVIDNVIVKKSEAETDQYNKSCWTGTTNSETQYNDTEGLLRYAFDNNPSTIWHSNWKGATDKLTGSNSFYAEIDMCQKYTINQFSFTPRTSQDSGQVTKADLYVKANANDEWKQVATDQVFEASRAKKTFMFDEQEVRYVKFVAKSSNDGWVAVSEFGVANKPSSTVRVFVAADPAEGGTVSVAAEGETGTDTAVDVASGASVTAKAVAADGYRFSGWFTTASETAVSTDATYTFAADGNTALTAKFTKDSTPDPGPKPTISSIAVTKPTVTDYKVGDTFDATGLAVTATMSDGSTKTLTAGEYTLSATQDGAAVALDKAFAKAGKVTVTVTANGKTATFDVTVTAKDPDPEPATLKSIKVTSKPDKTTYTVDETFAKTGLAVTGTWSDGKTALLKDGEYKLSAVDADGKTVDLTKPFTAAGDVTVTVTSGKLTDSFTITVKAKTVTPAPGDNKPGENKPGADKPKPNTPDEVAKLEHHHHHH

SEQ ID NO: 14 - protein, 1597 aa, Artificial Sequence
  Synthetic polypeptide (BbhI, D746A mutant, truncated construct including a His-tag)
MASMTGGQQMGRDPNSSSVDKLSDDNLALNQTVTASSYEVATTAPEKAVDGDLGTRWGTAQNKAANEWIEVGLGGTKTVKQINIDFERKDADQNITSFKVELKQGDTYTKVYQKDTRAKQQEIILLDQAQQASAVKVTVLSADGGTMNWVNVGINEISVYSAPKETVLDTADTNHMLGATMTASSNETATLTPDKAIDQNRTGRNNRWASGYETPSNIWLKAEFPRLTAVKDIRIYFFERDVNPKPTNVQSFDLSYTDSEGTEHTLKSGYAMTASGAGYVADVVIQLDQAVNARSLKLSNFAIKSSEYNNVSVAEWEAYSNDQAEPGATLDSVVSDLESNHLTIETDTDTLALPTVPDGYTVKFNGADYEQLIAADGTVNHPLVDKTVQVAYVVTDTATGNTKTTSDIPYVVKGTNQQQEGNNAKPTIIPEIAEWHSTSAAKLAASAVTKVVYDDDSLKAVVDEFVADYKDFTGIKLTAKKGAAEAGAFNFVKTDSTAAIAQLGDEGYTMDIRADRVVAKSSSVTGNMYAMQTILQMTKQDANGFVIGSMRDYPRFTTRGLLLDVARKPVSLEMMREITRTMRYYKMNDFQAHLSDNYIFLENYGKGDNEDEAFKAYDAFRLESSLTNDKGESPTAEDYSISKKTFKQFIQDERALGMNVVPEIDVPAHANSFTKIWPELMVKGRVSPINSNRPLIDHLDVSKPETIAKIKEIFDDYTKGDDPTFDSDTTVHIGAAEFLYNYTAYRKFINEIVPYIKDTNTVRMWGGLTWINDHKTEITKDAIENVEMNLWSKDWADGLQMYNMGYKLINTIDDYGYMVPNGSYGRANAYGDLLNISRVFDSFEPNKVRSSGGYQAVPSGDDQMLGAAFAIWSDNIDKSASGLTESDLYWRFFDAMPFYAEKTWAATGKEKGTAAKLTALAAKQGTGPRTNPYYQATSKNSVYESYDFNDGLADASGNGRDLTIGDGSKAAVKDQSLKLAGGSSYATSKLDKLGNGNELTFDVTLQQAAKPGDILFEADAPYGTHDIRVMENGKLGFTRELYNYYFDYELPVGKTVTVTIKVDQQTTKLYVDGEFVSDATGKYIDKGIEKKTGITAATFALPLQRIGSKTSAINGVIDNVIVKKSEAETDQYNKSCWTGTTNSETQYNDTEGLLRYAFDNNPSTIWHSNWKGATDKLTGSNSFYAEIDMCQKYTINQFSFTPRTSQDSGQVTKADLYVKANANDEWKQVATDQVFEASRAKKTFMFDEQEVRYVKFVAKSSNDGWVAVSEFGVANKPSSTVRVFVAADPAEGGTVSVAAEGETGTDTAVDVASGASVTAKAVAADGYRFSGWFTTASETAVSTDATYTFAADGNTALTAKFTKDSTPDPGPKPTISSIAVTKPTVTDYKVGDTFDATGLAVTATMSDGSTKTLTAGEYTLSATQDGAAVALDKAFAKAGKVTVTVTANGKTATFDVTVTAKDPDPEPATLKSIKVTSKPDKTTYTVDETFAKTGLAVTGTWSDGKTALLKDGEYKLSAVDADGKTVDLTKPFTAAGDVTVTVTSGKLTDSFTITVKAKTVTPAPGDNKPGENKPGADKPKPNTPDEVAKLEHHHHHH

SEQ ID NO: 15 - protein, 1597 aa, Artificial Sequence
  Synthetic polypeptide (BbhI, D746Q mutant, truncated construct including a His-tag)
MASMTGGQQMGRDPNSSSVDKLSDDNLALNQTVTASSYEVATTAPEKAVDGDLGTRWGTAQNKAANEWIEVGLGGTKTVKQINIDFERKDADQNITSFKVELKQGDTYTKVYQKDTRAKQQEIILLDQAQQASAVKVTVLSADGGTMNWVNVGINEISVYSAPKETVLDTADTNHMLGATMTASSNETATLTPDKAIDQNRTGRNNRWASGYETPSNIWLKAEFPRLTAVKDIRIYFFERDVNPKPTNVQSFDLSYTDSEGTEHTLKSGYAMTASGAGYVADVVIQLDQAVNARSLKLSNFAIKSSEYNNVSVAEWEAYSNDQAEPGATLDSVVSDLESNHLTIETDTDTLALPTVPDGYTVKFNGADYEQLIAADGTVNHPLVDKTVQVAYVVTDTATGNTKTTSDIPYVVKGTNQQQEGNNAKPTIIPEIAEWHSTSAAKLAASAVTKVVYDDDSLKAVVDEFVADYKDFTGIKLTAKKGAAEAGAFNFVKTDSTAAIAQLGDEGYTMDIRADRVVAKSSSVTGNMYAMQTILQMTKQDANGFVIGSMRDYPRFTTRGLLLDVARKPVSLEMMREITRTMRYYKMNDFQAHLSDNYIFLENYGKGDNEDEAFKAYDAFRLESSLTNDKGESPTAEDYSISKKTFKQFIQDERALGMNVVPEIDVPAHANSFTKIWPELMVKGRVSPINSNRPLIDHLDVSKPETIAKIKEIFDDYTKGDDPTFDSDTTVHIGAQEFLYNYTAYRKFINEIVPYIKDTNTVRMWGGLTWINDHKTEITKDAIENVEMNLWSKDWADGLQMYNMGYKLINTIDDYGYMVPNGSYGRANAYGDLLNISRVFDSFEPNKVRSSGGYQAVPSGDDQMLGAAFAIWSDNIDKSASGLTESDLYWRFFDAMPFYAEKTWAATGKEKGTAAKLTALAAKQGTGPRTNPYYQATSKNSVYESYDFNDGLADASGNGRDLTIGDGSKAAVKDQSLKLAGGSSYATSKLDKLGNGNELTFDVTLQQAAKPGDILFEADAPYGTHDIRVMENGKLGFTRELYNYYFDYELPVGKTVTVTIKVDQQTTKLYVDGEFVSDATGKYIDKGIEKKTGITAATFALPLQRIGSKTSAINGVIDNVIVKKSEAETDQYNKSCWTGTTNSETQYNDTEGLLRYAFDNNPSTIWHSNWKGATDKLTGSNSFYAEIDMCQKYTINQFSFTPRTSQDSGQVTKADLYVKANANDEWKQVATDQVFEASRAKKTFMFDEQEVRYVKFVAKSSNDGWVAVSEFGVANKPSSTVRVFVAADPAEGGTVSVAAEGETGTDTAVDVASGASVTAKAVAADGYRFSGWFTTASETAVSTDATYTFAADGNTALTAKFTKDSTPDPGPKPTISSIAVTKPTVTDYKVGDTFDATGLAVTATMSDGSTKTLTAGEYTLSATQDGAAVALDKAFAKAGKVTVTVTANGKTATFDVTVTAKDPDPEPATLKSIKVTSKPDKTTYTVDETFAKTGLAVTGTWSDGKTALLKDGEYKLSAVDADGKTVDLTKPFTAAGDVTVTVTSGKLTDSFTITVKAKTVTPAPGDNKPGENKPGADKPKPNTPDEVAKLEHHHHHH

SEQ ID NO: 16 - protein, 1597 aa, Artificial Sequence
  Synthetic polypeptide (BbhI, Y827F mutant, truncated construct including a His-tag)
MASMTGGQQMGRDPNSSSVDKLSDDNLALNQTVTASSYEVATTAPEKAVDGDLGTRWGTAQNKAANEWIEVGLGGTKTVKQINIDFERKDADQNITSFKVELKQGDTYTKVYQKDTRAKQQEIILLDQAQQASAVKVTVLSADGGTMNWVNVGINEISVYSAPKETVLDTADTNHMLGATMTASSNETATLTPDKAIDQNRTGRNNRWASGYETPSNIWLKAEFPRLTAVKDIRIYFFERDVNPKPTNVQSFDLSYTDSEGTEHTLKSGYAMTASGAGYVADVVIQLDQAVNARSLKLSNFAIKSSEYNNVSVAEWEAYSNDQAEPGATLDSVVSDLESNHLTIETDTDTLALPTVPDGYTVKFNGADYEQLIAADGTVNHPLVDKTVQVAYVVTDTATGNTKTTSDIPYVVKGTNQQQEGNNAKPTIIPEIAEWHSTSAAKLAASAVTKVVYDDDSLKAVVDEFVADYKDFTGIKLTAKKGAAEAGAFNFVKTDSTAAIAQLGDEGYTMDIRADRVVAKSSSVTGNMYAMQTILQMTKQDANGFVIGSMRDYPRFTTRGLLLDVARKPVSLEMMREITRTMRYYKMNDFQAHLSDNYIFLENYGKGDNEDEAFKAYDAFRLESSLTNDKGESPTAEDYSISKKTFKQFIQDERALGMNVVPEIDVPAHANSFTKIWPELMVKGRVSPINSNRPLIDHLDVSKPETIAKIKEIFDDYTKGDDPTFDSDTTVHIGADEFLYNYTAYRKFINEIVPYIKDTNTVRMWGGLTWINDHKTEITKDAIENVEMNLWSKDWADGLQMYNMGYKLINTIDDYGFMVPNGSYGRANAYGDLLNISRVFDSFEPNKVRSSGGYQAVPSGDDQMLGAAFAIWSDNIDKSASGLTESDLYWRFFDAMPFYAEKTWAATGKEKGTAAKLTALAAKQGTGPRTNPYYQATSKNSVYESYDFNDGLADASGNGRDLTIGDGSKAAVKDQSLKLAGGSSYATSKLDKLGNGNELTFDVTLQQAAKPGDILFEADAPYGTHDIRVMENGKLGFTRELYNYYFDYELPVGKTVTVTIKVDQQTTKLYVDGEFVSDATGKYIDKGIEKKTGITAATFALPLQRIGSKTSAINGVIDNVIVKKSEAETDQYNKSCWTGTTNSETQYNDTEGLLRYAFDNNPSTIWHSNWKGATDKLTGSNSFYAEIDMCQKYTINQFSFTPRTSQDSGQVTKADLYVKANANDEWKQVATDQVFEASRAKKTFMFDEQEVRYVKFVAKSSNDGWVAVSEFGVANKPSSTVRVFVAADPAEGGTVSVAAEGETGTDTAVDVASGASVTAKAVAADGYRFSGWFTTASETAVSTDATYTFAADGNTALTAKFTKDSTPDPGPKPTISSIAVTKPTVTDYKVGDTFDATGLAVTATMSDGSTKTLTAGEYTLSATQDGAAVALDKAFAKAGKVTVTVTANGKTATFDVTVTAKDPDPEPATLKSIKVTSKPDKTTYTVDETFAKTGLAVTGTWSDGKTALLKDGEYKLSAVDADGKTVDLTKPFTAAGDVTVTVTSGKLTDSFTITVKAKTVTPAPGDNKPGENKPGADKPKPNTPDEVAKLEHHHHHH

SEQ ID NO: 17 - protein, 1039 aa, Artificial Sequence
  Synthetic polypeptide (LnbB, wild type (wt), truncated construct including a His-tag)
MADDSAAGYSATAPVNLTRPATVPSMDGWTDGTGAWTLGEGTRVVSSDALAARAQSLASELTKFTDVDIKAATGSATGKDISLTLDASKKAELGDEGFKLNIGSKGLEVIGATDIGVFYGTRSVSQMLRQGQLTLPAGTVATKPKYKERGATLCACQINISTDWIDRFLSDMADLRLNYVLLEMKLKPEEDNTKKAATWSYYTRDDVKKFVKKANNYGIDVIPEINSPGHMNVWLENYPEYQLADNSGRKDPNKLDISNPEAVKFYKTLIDEYDGVFTTKYWHMGADEYMIGTSFDNYSKLKTFAEKQYGAGATPNDAFTGFINDIDKYVKAKGKQLRIWNDGIVNTKNVSLNKDIVIEYWYGAGRKPQELVQDGYTLMNATQALYWSRSAQVYKVNAARLYNNNWNVGTFDGGRQIDKNYDKLTGAKVSIWPDSSYFQTENEVEKEIFDGMRFISQMTWSDSRPWATWNDMKADIDKIGYPLDIREYDYTPVDAGIYDIPQLKSISKGPWELITTPDGYYQMKDTVSGKCLALFTGSKHLDVVTQVGARPELRNCADVSVGQDQRNTANERNTQKWQIRADKDGKYTISPALTQQRLAIATGNEQNIDLETHRPAAGTVAQFPADLVSDNALFTLTGHMGMSATVDSKTVNPASPSKITVKVRAASNANTGDVTVTPVVPEGWEIKPGSVSLKSIPAGKAAIAYFNVVNTTGTGDATVQFKLTNTKTGEELGTTSVALTGSLTKDVEASDYAASSQETTGEHAPVGNAFDKNANTFWHSKYSNPSANLPHWLAFKASPGEGNKIAAITHLYRQDKLNGPAKNVAVYVVAASDANSVADVTNWGEPVATAEFPYTKELQTIALPNTIPSGDVYVKFQINDAWGLTETSAGVTWAAVAELAATAKATPVELTEPEQPKDNPEVTETPEATGVTVSGDGVANGALSLKKGTTAQLTAKVAPDDADQAVTWASSDDKVVTVDKTGKVTAVAKGVAKVTATTANGKSASVTVTVTEDSEVPGPTGPTEPTKPGTELEHHHHHH

SEQ ID NO: 18 - protein, 1039 aa, Artificial Sequence
  Synthetic polypeptide (LnbB, D320E mutant, truncated construct including a His-tag)
MADDSAAGYSATAPVNLTRPATVPSMDGWTDGTGAWTLGEGTRVVSSDALAARAQSLASELTKFTDVDIKAATGSATGKDISLTLDASKKAELGDEGFKLNIGSKGLEVIGATDIGVFYGTRSVSQMLRQGQLTLPAGTVATKPKYKERGATLCACQINISTDWIDRFLSDMADLRLNYVLLEMKLKPEEDNTKKAATWSYYTRDDVKKFVKKANNYGIDVIPEINSPGHMNVWLENYPEYQLADNSGRKDPNKLDISNPEAVKFYKTLIDEYDGVFTTKYWHMGAEEYMIGTSFDNYSKLKTFAEKQYGAGATPNDAFTGFINDIDKYVKAKGKQLRIWNDGIVNTKNVSLNKDIVIEYWYGAGRKPQELVQDGYTLMNATQALYWSRSAQVYKVNAARLYNNNWNVGTFDGGRQIDKNYDKLTGAKVSIWPDSSYFQTENEVEKEIFDGMRFISQMTWSDSRPWATWNDMKADIDKIGYPLDIREYDYTPVDAGIYDIPQLKSISKGPWELITTPDGYYQMKDTVSGKCLALFTGSKHLDVVTQVGARPELRNCADVSVGQDQRNTANERNTQKWQIRADKDGKYTISPALTQQRLAIATGNEQNIDLETHRPAAGTVAQFPADLVSDNALFTLTGHMGMSATVDSKTVNPASPSKITVKVRAASNANTGDVTVTPVVPEGWEIKPGSVSLKSIPAGKAAIAYFNVVNTTGTGDATVQFKLTNTKTGEELGTTSVALTGSLTKDVEASDYAASSQETTGEHAPVGNAFDKNANTFWHSKYSNPSANLPHWLAFKASPGEGNKIAAITHLYRQDKLNGPAKNVAVYVVAASDANSVADVTNWGEPVATAEFPYTKELQTIALPNTIPSGDVYVKFQINDAWGLTETSAGVTWAAVAELAATAKATPVELTEPEQPKDNPEVTETPEATGVTVSGDGVANGALSLKKGTTAQLTAKVAPDDADQAVTWASSDDKVVTVDKTGKVTAVAKGVAKVTATTANGKSASVTVTVTEDSEVPGPTGPTEPTKPGTELEHHHHHH

SEQ ID NO: 19 - protein, 1039 aa, Artificial Sequence
  Synthetic polypeptide (LnbB, D320A mutant, truncated construct including a His-tag)
MADDSAAGYSATAPVNLTRPATVPSMDGWTDGTGAWTLGEGTRVVSSDALAARAQSLASELTKFTDVDIKAATGSATGKDISLTLDASKKAELGDEGFKLNIGSKGLEVIGATDIGVFYGTRSVSQMLRQGQLTLPAGTVATKPKYKERGATLCACQINISTDWIDRFLSDMADLRLNYVLLEMKLKPEEDNTKKAATWSYYTRDDVKKFVKKANNYGIDVIPEINSPGHMNVWLENYPEYQLADNSGRKDPNKLDISNPEAVKFYKTLIDEYDGVFTTKYWHMGAAEYMIGTSFDNYSKLKTFAEKQYGAGATPNDAFTGFINDIDKYVKAKGKQLRIWNDGIVNTKNVSLNKDIVIEYWYGAGRKPQELVQDGYTLMNATQALYWSRSAQVYKVNAARLYNNNWNVGTFDGGRQIDKNYDKLTGAKVSIWPDSSYFQTENEVEKEIFDGMRFISQMTWSDSRPWATWNDMKADIDKIGYPLDIREYDYTPVDAGIYDIPQLKSISKGPWELITTPDGYYQMKDTVSGKCLALFTGSKHLDVVTQVGARPELRNCADVSVGQDQRNTANERNTQKWQIRADKDGKYTISPALTQQRLAIATGNEQNIDLETHRPAAGTVAQFPADLVSDNALFTLTGHMGMSATVDSKTVNPASPSKITVKVRAASNANTGDVTVTPVVPEGWEIKPGSVSLKSIPAGKAAIAYFNVVNTTGTGDATVQFKLTNTKTGEELGTTSVALTGSLTKDVEASDYAASSQETTGEHAPVGNAFDKNANTFWHSKYSNPSANLPHWLAFKASPGEGNKIAAITHLYRQDKLNGPAKNVAVYVVAASDANSVADVTNWGEPVATAEFPYTKELQTIALPNTIPSGDVYVKFQINDAWGLTETSAGVTWAAVAELAATAKATPVELTEPEQPKDNPEVTETPEATGVTVSGDGVANGALSLKKGTTAQLTAKVAPDDADQAVTWASSDDKVVTVDKTGKVTAVAKGVAKVTATTANGKSASVTVTVTEDSEVPGPTGPTEPTKPGTELEHHHHHH

SEQ ID NO: 20 - protein, 1039 aa, Artificial Sequence
  Synthetic polypeptide (LnbB, Y419F mutant, truncated construct including a His-tag)
MADDSAAGYSATAPVNLTRPATVPSMDGWTDGTGAWTLGEGTRVVSSDALAARAQSLASELTKFTDVDIKAATGSATGKDISLTLDASKKAELGDEGFKLNIGSKGLEVIGATDIGVFYGTRSVSQMLRQGQLTLPAGTVATKPKYKERGATLCACQINISTDWIDRFLSDMADLRLNYVLLEMKLKPEEDNTKKAATWSYYTRDDVKKFVKKANNYGIDVIPEINSPGHMNVWLENYPEYQLADNSGRKDPNKLDISNPEAVKFYKTLIDEYDGVFTTKYWHMGADEYMIGTSFDNYSKLKTFAEKQYGAGATPNDAFTGFINDIDKYVKAKGKQLRIWNDGIVNTKNVSLNKDIVIEYWYGAGRKPQELVQDGYTLMNATQALFWSRSAQVYKVNAARLYNNNWNVGTFDGGRQIDKNYDKLTGAKVSIWPDSSYFQTENEVEKEIFDGMRFISQMTWSDSRPWATWNDMKADIDKIGYPLDIREYDYTPVDAGIYDIPQLKSISKGPWELITTPDGYYQMKDTVSGKCLALFTGSKHLDVVTQVGARPELRNCADVSVGQDQRNTANERNTQKWQIRADKDGKYTISPALTQQRLAIATGNEQNIDLETHRPAAGTVAQFPADLVSDNALFTLTGHMGMSATVDSKTVNPASPSKITVKVRAASNANTGDVTVTPVVPEGWEIKPGSVSLKSIPAGKAAIAYFNVVNTTGTGDATVQFKLTNTKTGEELGTTSVALTGSLTKDVEASDYAASSQETTGEHAPVGNAFDKNANTFWHSKYSNPSANLPHWLAFKASPGEGNKIAAITHLYRQDKLNGPAKNVAVYVVAASDANSVADVTNWGEPVATAEFPYTKELQTIALPNTIPSGDVYVKFQINDAWGLTETSAGVTWAAVAELAATAKATPVELTEPEQPKDNPEVTETPEATGVTVSGDGVANGALSLKKGTTAQLTAKVAPDDADQAVTWASSDDKVVTVDKTGKVTAVAKGVAKVTATTANGKSASVTVTVTEDSEVPGPTGPTEPTKPGTELEHHHHHH

SEQ ID NO: 21 - protein, 494 aa, Unknown
  uncultured Actinomycetales bacterium; GenBank ACK34128.1
MSPVMNTVIPRPSSVLGTGRSFELPADARILVQPNAPEVAQIGEFLAAALRPATGLALPVTAGAAQERGTIHLTTAAADPALGAEGYELLVAPHGVLLAAPRPAGLFRGVQTIRQLLPAAVERRTVAPGPWTLPTGVLRDAPRFAWRGAMLDVARHFFPVADVKRYLDLLAYYKFNVLHLHLTDDQGWRIEIKSWPRLAEYGGSTAVDGAPGGYYTQEQYADLARYAAERYITIVPEIDMPSHTNAALASYALLNCEGEAPALYTGTAVGFSTLCMGKETTFRFMDDVLGELAALTPGPYLHIGGDEADSTSPEDYQTFMRQVQAIVQSHGKTVVGWGEIAQAPLTPGTMVQHWRQDLTPQAAASGHKVILSPAGKTYLDMKYDEATPFGLSWAGYVTVQDAFAWNPGAYLAGVGEDAVAGVEAPLWTETIPTFAEAEFMLFPRLLAVAELGWSPAAGRTWDEFRSRLAHHGPRLEGLGVNFFRAPEIDWPETL

SEQ ID NO: 22 - protein, 537 aa, Unknown
  uncultured Bacteroidetes bacterium, GenBank AKC34129.1
MKTIASLLIAGSLATSAAEPPALIPQPVSVQPGEGSFKFSAGTGIRHDRALESEAKLLASDLAKLTDSQPKTVVKELTNALPSEILLDFSEKTDLPPSGYELKVQPNGVVIRGKDAAGVFLGTRTLLQLLPAKLAEGSAAIPAVTITDYPRFAWRGMMLDVGRHFHPVPDIKRFIDWMAFHKLNSFHWHLTEDQGWRIEIKKYPKLTEVGAFRESSPPYGNRNSDDGVRYGGFYTQEQLKDVVAYAAARHITVVPEIEMPGHAAAAIAAYPELGNTDIPGYSPKVMTRWGVHPYIFSPKEETFTFLEDVLSEVCDLFPSKYIHIGGDEAPKDQWKQSKFAQEVIKREGLKNEEELQSWFIRRIGRFLESKNRNLIGWDEIQEGGLPKTATMMVWRDAKWAKHALSLGNNVVMATTSHTYLDYYQNPAATELAKGVEYEAIGGHLPLEKVYSYNPTFVAENPQQEKQILGTQAQLWSEYFKDMKKVEYHAFPRIAALAEVAWTPLALKNFDGFSKRLEGIMQHYEAGNLHYCKPSTEK

SEQ ID NO: 23 - protein, 885 aa, Serratia marcescens
MNAFKLSALARLTATMGFLGGMGSAMADQQLVDQLSQLKLNVKMLDNRAGENGVDCAALGADWASCNRVLFTLSNDGQAIDGKDWVIYFHSPRQTLRVDNDQFKIAHLTGDLYKLEPTAKFSGFPAGKAVEIPVVAEYWQLFRNDFLPRWYATSGDAKPKMLANTDTENLDQFVAPFTGDQWKRTKDDKNILMTPASRFVSNADLQTLPAGALRGKIVPTPMQVKVHAQDADLRKGVALDLSTLVKPAADVVSQRFALLGVPVQTNGYPIKTDIQPGKFKGAMAVSGAYELKIGKKEAQVIGFDQAGVFYGLQSILSLVPSDGSGKIATLDASDAPRFPYRGIFLDVARNFHKKDAVLRLLDQMAAYKLNKFHFHLSDDEGWRIEIPGLPELTEVGGQRCHDLSETTCLLPQYGQGPDVYGGFFSRQDYIDIIKYAQARQIEVIPEIDMPAHARAAVVSMEARYKKLHAAGKEQEANEFRLVDPTDTSNTTSVQFFNRQSYLNPCLDSSQRFVDKVIGEIAQMHKEAGQPIKTWHFGGDEAKNIRLGAGYTDKAKPEPGKGIIDQSNEDKPWAKSQVCQTMIKEGKVADMEHLPSYFGQEVSKLVKAHGIDRMQAWQDGLKDAESSKAFATSRVGVNFWDTLYWGGFDSVNDWANKGYEVVVSNPDYVYMDFPYEVNPDERGYYWGTRFSDERKVFSFAPDNMPQNAETSVDRDGNHFNAKSDKPWPGAYGLSAQLWSETQRTDPQMEYMIFPRALSVAERSWHRAGWEQDYRAGREYKGGETHFVDTQALEKDWLRFANILGQRELAKLDKGGVAYRLPVPGARVAAGKLEANIALPGLGIEYSTDGGKQWQRYDAKAKPAVSGEVQVRSVSPDGKRYSRAEKV

SEQ ID NO: 24 - protein, 506 aa, Streptomyces plicatus
MTTGAAPDRKAPVRPTPLDRVIPAPASVDPGGAPYRITRGTHIRVDDSREARRVGDYLADLLRPATGYRLPVTAHGHGGIRLRLAGGPYGDEGYRLDSGPAGVTITARKAAGLFHGVQTLRQLLPPAVEKDSAQPGPWLVAGGTIEDTPRYAWRSAMLDVSRHFFGVDEVKRYIDRVARYKYNKLHLHLSDDQGWRIAIDSWPRLATYGGSTEVGGGPGGYYTKAEYKEIVRYAASRHLEVVPEIDMPGHTNAALASYAELNCDGVAPPLYTGTKVGFSSLCVDKDVTYDFVDDVIGELAALTPGRYLHIGGDEAHSTPKADFVAFMKRVQPIVAKYGKTVVGWHQLAGAEPVEGALVQYWGLDRTGDAEKAEVAEAARNGTGLILSPADRTYLDMKYTKDTPLGLSWAGYVEVQRSYDWDPAGYLPGAPADAVRGVEAPLWTETLSDPDQLDYMAFPRLPGVAELGWSPASTHDWDTYKVRLAAQAPYWEAAGIDFYRSPQVPWT

SEQ ID NO: 25 - protein, 737 aa, Artificial Sequence
  Synthetic polypeptide (Consensus sequence of alignment in Figure 16)
  misc_feature (7)..(7): Xaa can be any naturally occurring amino acid
  misc_feature (11)..(13): Xaa can be any naturally occurring amino acid
  misc_feature (21)..(22): Xaa can be any naturally occurring amino acid
  misc_feature (34)..(34): Xaa can be any naturally occurring amino acid
  misc_feature (40)..(40): Xaa can be any naturally occurring amino acid
  misc_feature (42)..(42): Xaa can be any naturally occurring amino acid
  misc_feature (45)..(45): Xaa can be any naturally occurring amino acid
  misc_feature (51)..(51): Xaa can be any naturally occurring amino acid
  misc_feature (53)..(53): Xaa can be any naturally occurring amino acid
  misc_feature (55)..(55): Xaa can be any naturally occurring amino acid
  misc_feature (65)..(65): Xaa can be any naturally occurring amino acid
  misc_feature (72)..(72): Xaa can be any naturally occurring amino acid
  misc_feature (74)..(74): Xaa can be any naturally occurring amino acid
  misc_feature (79)..(79): Xaa can be any naturally occurring amino acid
  misc_feature (84)..(84): Xaa can be any naturally occurring amino acid
  misc_feature (86)..(86): Xaa can be any naturally occurring amino acid
  misc_feature (107)..(107): Xaa can be any naturally occurring amino acid
  misc_feature (111)..(112): Xaa can be any naturally occurring amino acid
  misc_feature (123)..(123): Xaa can be any naturally occurring amino acid
  misc_feature (137)..(137): Xaa can be any naturally occurring amino acid
  misc_feature (139)..(139): Xaa can be any naturally occurring amino acid
  misc_feature (165)..(165): Xaa can be any naturally occurring amino acid
  misc_feature (168)..(168): Xaa can be any naturally occurring amino acid
  misc_feature (210)..(210): Xaa can be any naturally occurring amino acid
  misc_feature (215)..(216): Xaa can be any naturally occurring amino acid
  misc_feature (233)..(233): Xaa can be any naturally occurring amino acid
  misc_feature (247)..(247): Xaa can be any naturally occurring amino acid
  misc_feature (252)..(252): Xaa can be any naturally occurring amino acid
  misc_feature (287)..(287): Xaa can be any naturally occurring amino acid
  misc_feature (299)..(299): Xaa can be any naturally occurring amino acid
  misc_feature (317)..(318): Xaa can be any naturally occurring amino acid
  misc_feature (337)..(337): Xaa can be any naturally occurring amino acid
  misc_feature (346)..(346): Xaa can be any naturally occurring amino acid
  misc_feature (350)..(350): Xaa can be any naturally occurring amino acid
  misc_feature (368)..(368): Xaa can be any naturally occurring amino acid
  misc_feature (385)..(385): Xaa can be any naturally occurring amino acid
  misc_feature (398)..(398): Xaa can be any naturally occurring amino acid
  misc_feature (426)..(426): Xaa can be any naturally occurring amino acid
  misc_feature (429)..(429): Xaa can be any naturally occurring amino acid
  misc_feature (435)..(435): Xaa can be any naturally occurring amino acid
  misc_feature (549)..(549): Xaa can be any naturally occurring amino acid
  misc_feature (557)..(557): Xaa can be any naturally occurring amino acid
  misc_feature (608)..(608): Xaa can be any naturally occurring amino acid
  misc_feature (610)..(611): Xaa can be any naturally occurring amino acid
  misc_feature (639)..(639): Xaa can be any naturally occurring amino acid
  misc_feature (642)..(642): Xaa can be any naturally occurring amino acid
  misc_feature (645)..(645): Xaa can be any naturally occurring amino acid
  misc_feature (647)..(647): Xaa can be any naturally occurring amino acid
  misc_feature (693)..(693): Xaa can be any naturally occurring amino acid
  misc_feature (711)..(711): Xaa can be any naturally occurring amino acid
  misc_feature (729)..(729): Xaa can be any naturally occurring amino acid
MASMTGXGQMXXXPVNVPRPXXVQSFDSGYASGXGYEWTXPXGYXVVDDDXLXAXVDEFVADLKXFTGIKLXAXKGAAXAGAFXVXLDSSAAAQLGDEGYTLDIGAXGVVXXGSSVTGVFYGXQTILQMLKQDANGXPXGTMRDYPRFTTRGALLDVARKPVSEXMRXITRMAYYKLNDFQAHLSDNEWLENYGKGDNEDEAAYFYTKKXFKQFXXDARALGINVVPEIDVPXHANSFLKIYPELMXDGRVXPPLIDHLDVSKPETIAFIKEIIDEYDPTFTSDKYXHIGADEFLYNYXATPDYRKFINEIVPYVKXXGVRMVIWGGLTWINDEITXDGIENVEMXLWSXDWADQMYNMGYKLINTIXDYGYMVPDGSYGRANPXGWDLLNISRVFDXWEPGGYQAVPGDDQVLGAAFALWSDNIXTDXDLYYRXFPALPFVAEKGWAAKPGDILFEAAPAGTHDIRYFDVSDATGTGITAATFALPLQRIGSKTSAINGVIDNVIVKKSEATGTTNSETQYNDTEGLLRYAFDNNPSTIWHSNYKGAXDKLTGSNXHYYTINQFSFTPRTSQDSGQVTKADLYVKANANDEWKQVATSSNDGWVAVXEXXVANKPSSAKFTKDSTPDPGPKAVTKPEXTGXTFXAXGLAVTATMSDGSTKTLTAGEAPQDPDKTTTWSDGKTALLKDGEYKXSAVDADGKTVDLTKPFTXAGDVPGDNKPGENKPGTXLEHHHHHH

SEQ ID NO: 26 - protein, 43 aa, Artificial Sequence
  Synthetic polypeptide
TTTTTTAAAAAAAACCCCHHHCCCCAAARVHHHHHTTTTTTTT

SEQ ID NO: 27 - protein, 36 aa, Artificial Sequence
  Synthetic polypeptide
TTAAAAAAAACCCCHHCCCCAAADLSSHHHHHTTTT